(19) United States
(12) Patent Application Publication
(10) Pub. No.: US 2026/0306641 A1
(43) Pub. Date: Oct. 8, 2026

(54) HELICASE DEGRADERS

(71) Applicant: H. LEE MOFFITT CANCER CENTER AND RESEARCH INSTITUTE, INC., Tampa, FL (US)

(72) Inventors: Mark ALEXANDROW, Lutz, FL (US); Harshani LAWRENCE, Lutz, FL (US); Nicholas LAWRENCE, Lutz, FL (US)

(21) Appl. No.: 19/477,592

(22) PCT Filed: Apr. 22, 2024
(86) PCT No.: PCT/US2024/025657

§ 371 Date: Oct. 22, 2025

Related U.S. Application Data
(60) Provisional application No. 63/461,246, filed on Apr. 22, 2023.
(60) Provisional application No. 63/620,246, filed on Jan. 12, 2024.

(51) Int. Cl. A61K 47/55; A61K 45/06; A61K 47/54; A61P 35/00

(52) U.S. Cl. CPC A61K 47/55; A61K 45/06; A61K 47/545; A61K 47/549; A61P 35/00

(57) ABSTRACT

This disclosure provides compounds useful for the treatment of medical disorders, such as cancers, and more particularly Oto degraders of helicases, such as Superfamily 3 (SF3) and Superfamily 6 (SF6) helicases.

### CROSS-REFERENCE TO RELATED APPLICATIONS

This application claims the benefit of priority to U.S. Provisional Application No. 63/461,246 filed Apr. 22, 2023, and U.S. Provisional Application No. 63/620,246 filed Jan. 12, 2024, the disclosures of which are incorporated herein by reference in their entireties.

### STATEMENT REGARDING FEDERALLY SPONSORED RESEARCH OR DEVELOPMENT

This invention was made with government support under Grant Nos. P30-CA076292, R50CA211447, and GM140140 awarded by the National Institutes of Health. The Government has certain rights in the invention.

### TECHNICAL FIELD

This disclosure relates to compounds for the treatment of medical disorders, such as cancers, and more particularly to degraders of helicases, such as Superfamily 3 (SF3) and Superfamily 6 (SF6) helicases.

### BACKGROUND

Helicases are a class of enzymes that unpack an organism's genes. They are motor proteins that move directionally along a nucleic acid phosphodiester backbone, separating two annealed nucleic acid strands using energy from ATP hydrolysis. There are many helicases representing the great variety of processes in which strand separation must be catalyzed, such as DNA replication, transcription, translation, recombination, DNA repair, and ribosome biogenesis. Helicases are classified into six superfamilies based on their shared sequence motifs; helicases not forming a ring structure are in superfamilies 1 and 2, and ring-forming helicases form part of superfamilies 3 to 6. In particular, superfamily 3 (SF3) consists of AAA+ helicases encoded mainly by small DNA viruses and some large nucleocytoplasmic DNA viruses, with the most known being the papilloma virus E1 helicase. Superfamily 6 (SF6) contains the core AAA+ that are not included in the SF3 classification; some proteins in this group are mini chromosome maintenance MCM, CMG, RuvB, RuvA, and RuvC.

The human replicative helicase functions during DNA replication to melt double-stranded DNA (dsDNA), allowing the polymerases and other replisome components access to single-stranded DNA (ssDNA) for synthesis of daughter strands of DNA. The human replicative helicase is referred to as the CMG helicase, which is derived from the names of its core subunits: Cdc45-MCM complex-GINS complex. MCM subunits are the six Mcm2-7 proteins, and GINS subunits are the four proteins named Go-Ichi-Ni-San (Japanese for 5-1-2-3). The entire CMG holoenzyme is thus composed of 11 subunits, Cdc45, 6 MCM monomers, and 4 GINS subunits, producing a very large ~750-800 kDa enzyme. Enzymatic activity of the CMG helicase is derived from ATP binding and hydrolysis within the regions between each pair of MCM dimers that make up the MCM hexamer. There are six pairs of MCMs that form six distinct ATP hydrolysis clefts, which work in a non-symmetrical and combinatorial manner to hydrolyze ATP and alter MCM subunit structures to achieve movement along DNA during the melting steps. ATP hydrolysis alters a staircase structure within the central channel of the MCM hexamer through which ssDNA moves in response to changes to this staircase structure during inter-coordinated ATP hydrolysis steps between MCM subunits.

The CMG helicase is an attractive target for cancer drug development due to unique features of its assembly, utilization in cells, and oncogene-induced errors in CMG management that lead to replication dysfunction during tumorigenesis and chemotherapeutic intervention. During the cell cycle mammalian cells are ‘smart’ and predict that they will likely encounter problems during the replicative S-phase, when DNA is being duplicated. For this reason, mammalian cells assemble more CMG helicase precursors, the MCM hexamer, than will be required to complete a normal unperturbed S-phase. An excess of reserve MCM hexamers (~5× needed) are pre-loaded onto DNA prior to S-phase beginning. Only a subset of these MCM hexamers are chosen, apparently stochastically, to become full CMG helicases upon recruitment of Cdc45 and GINS subunits. Those CMG helicases that form are active during DNA replication. However, if the cell encounters problems such as fork stalling events due to heterochromatin resistance or chemotherapeutic drug exposure that stall forks, then the excess (reserve) MCM hexamers become converted to functional CMG helicases to recover from the fork stalling events. The previous CMG helicases that had been functioning stop unwinding DNA, and these new CMGs become necessary to complete S-phase and the cell cycle. Herein lies the problem in cancers: tumor cells have been found to lack a proper number of unused reserve MCM hexamers and cannot easily create new CMG helicases as needed, for example, in recovering from chemotherapy drugs.

There are two currently known mechanisms by which tumor cells mismanage MCM/CMG helicases and thus fail to contain enough reserve helicases. First, overexpression of Cyclin E, which is oncogenic for a number of tumor types, leads to a reduction in MCM hexamer loading onto DNA in tumor cells. This results in a lower yield of total MCM hexamers that could become CMGs for replication or recovery (relative to non-tumor cells that load normal levels of MCMs). Second, Myc overexpression, which is known to occur in 70% or more of human malignancies, produces a related but different effect. Myc is known to be involved in stimulating the assembly and activation of CMG helicases (from MCM hexamers), but too much Myc causes this process to become deregulated and leads to excessive CMG helicase activation. This extra activation of CMG helicases by Myc leads to a loss of unused reserve MCMs, as they have already been turned on by the excess Myc proteins. When a tumor cell with excessive Myc and overactive CMGs is exposed to fork stalling chemotherapy, there are not enough unused reserve MCMs available to mount a healthy response to allow survival of the tumor cells. Again, non-tumor cells do not have elevated Myc expression and CMG activation. Therefore, these two known mechanisms by which oncogenes (Cyclin E or Myc) can mismanage MCM/CMG complexes produce a tumor-selective weakness in CMG levels and recovery from replicative stresses such as fork stalling chemotherapy. These findings also argue that a therapeutic window exists between tumor cells and non-tumor cells in a likely poor response of tumor cells to chemotherapy drugs (for example, as combination approaches using a future CMG inhibitor and chemotherapy). It is predicted from these findings that future CMG inhibitors could provide a unique means of cancer intervention for a variety of cancer types since the CMG helicase presents an exploitable tumor-specific vulnerability. Note also that other oncogenes besides Myc and Cyclin E could be found to mismanage CMG dynamics in tumor cells, so this concept could extend beyond just these two examples.

Papillomaviridae is a family of non-enveloped DNA viruses whose members are known as papillomaviruses. Several hundred species of papillomaviruses have been identified, infecting all carefully inspected mammals as well as other vertebrates such as birds, snakes, turtles, and fish. Infection by most papillomavirus types is either asymptomatic or causes small benign tumors, known a papillomas or warts. Papillomas caused by some papillomavirus types carry a risk of becoming cancerous. Papillomaviruses replicate exclusively in the basal layer of the body surface tissues, with all known papillomavirus types infecting a particular body surface, typically the skin or mucosal epithelium of the genitals, anus, mouth, or airways. Papillomaviruses replicate exclusively in keratinocytes, with less-differentiated keratinocyte stem cells thought to be the initial target of productive papillomavirus infections. Subsequent steps in the viral life cycle are strictly dependent on the process of keratinocyte differentiation.

E1, an ATP-dependent DNA helicase, is the only enzyme encoded by papillomaviruses. It is essential for replication and amplification of the viral episome in the nucleus of infected cells. It forms a complex with the viral E2 protein, which is a site-specific DNA-binding transcriptional activator. The E1-E2 complex binds to the replication origin, which contains binding sites for both proteins. In addition to E2, it also interacts with DNA polymerase alpha and replication protein A to effect DNA replication. In solution, E1 is a monomer but binds DNA as a dimer. Recruitment of more E1 subunits to the complex leads to melting of the origin and ultimately to the formation of an E1 hexamer with helicase activity.

Human papillomavirus (HPV) infection is caused by HPV, a DNA virus of the Papillomaviridae family. About 90% of HPV infections cause no symptoms and resolve spontaneously within two years. In some cases, an HPV infection persists and results in either warts or precancerous lesions. These lesions, depending on the site affected, increase the risk of cancer of the cervix, vulva, vagina, penis, anus, mouth, or throat. Over 170 HPV types have been described, with more than 40 able to be spread through sexual contact and infect the anus and genitals. Nearly every individual is infected by HPV at some point in their lives, leading it to be the most common sexually transmitted infection globally.

### SUMMARY

The present disclosure provides compounds that are useful in the treatment of medical disorders. More particularly, compounds are provided that are degraders of helicases, such as Superfamily 3 (SF3) and Superfamily 6 (SF6) helicases, which are useful in the treatment of medical disorders such as cancers.

In one aspect, a compound of Formula I is provided

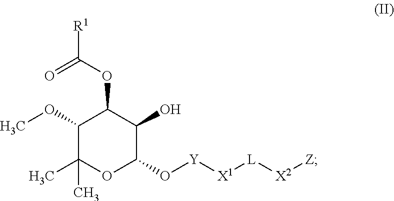

or a pharmaceutically acceptable salt thereof, wherein all variables are as defined herein.

In another aspect, a pharmaceutical composition is provided comprising a compound described herein, or a pharmaceutically acceptable salt thereof, and a pharmaceutically acceptable carrier or excipient.

In another aspect, a method is provided for treating cancer in a subject in need thereof comprising administering to the subject a therapeutically effective amount of a compound described herein, or a pharmaceutically acceptable salt thereof, or a pharmaceutical composition described herein.

In another aspect, a method is provided for treating cancer in a subject in need thereof comprising:
(a) determining whether the cancer is associated with elevated expression of Myc and/or elevated expression of Cyclin E; and
(b) if the cancer is determined to be associated with elevated expression of Myc and/or elevated expression of Cyclin E in (a), administering a therapeutically effective amount of a compound described herein, or a pharmaceutically acceptable salt thereof, or a pharmaceutical composition described herein.

In another aspect, a method is provided for treating cancer in a subject in need thereof, wherein the cancer has been previously determined to be associated with elevated expression of Myc and/or elevated expression of Cyclin E, the method comprising administering a therapeutically effective amount of a compound described herein, or a pharmaceutically acceptable salt thereof, or a pharmaceutical composition described herein.

In another aspect, a method is provided of treating an infection with a papillomavirus in a subject in need thereof comprising administering a therapeutically effective amount of a compound described herein, or a pharmaceutically acceptable salt thereof, or a pharmaceutical composition described herein.

In another aspect, a method is provided for inhibiting and/or degrading a helicase in a eukaryotic cell comprising contacting the cell with an effective amount of a compound described herein, or a pharmaceutically acceptable salt thereof.

In another aspect, a method is provided for inhibiting replication of a papillomavirus in a eukaryotic cell comprising contacting the cell with an effective amount of a compound described herein, or a pharmaceutically acceptable salt thereof.

In another aspect, a method is provided for treating cancer in a subject in need thereof comprising:
(a) determining whether the cancer harbors one or more inherited or acquired germ-line mutations; and
(b) if the cancer is determined to harbor one or more inherited or acquired germ-line mutations in (a), administering to the subject a therapeutically effective amount of a compound described herein, or a pharmaceutically acceptable salt thereof, or a pharmaceutical composition described herein.

In another aspect, a method is provided for treating cancer in a subject in need thereof, wherein the cancer has been previously determined to harbor one or more inherited or acquired germ-line mutations, the method comprising administering to the subject a therapeutically effective amount of a compound described herein, or a pharmaceutically acceptable salt thereof, or a pharmaceutical composition described herein.

In another aspect, a method is provided for treating cancer in a subject in need thereof comprising:
(a) administering to the subject a therapeutically effective amount of a compound described herein, or a pharmaceutically acceptable salt thereof, or a pharmaceutical composition described herein; and
(b) administering an additional therapeutic agent selected from a Chk1 inhibitor, an ATR inhibitor, a Cdc7 inhibitor, and a Parp inhibitor.

The details of one or more aspects of the disclosure are set forth in the accompanying drawings and the description below. Other features, objects, and advantages of the disclosure will be apparent from the description and the claims.

### DESCRIPTION OF DRAWINGS

FIG. 1 is a helicase assay (fork-unwinding; strang displacement assay) which shows that RF1-134 inhibits hCMG helixase activity 25% compared to CA1 (100% inhibition). For 20 uL reactions: 1 uL DMSO or drugs, 2 uL hCMG (~15 fmol enzyme), and 0.5 mM ATP were added. The noviose sugar (purple box; called RF1-134) was generated using synthetic chemical approaches. The sugar group alone can partially inhibit the hCMG, versus the complete CA1 compound which inhibits all hMG activity. CA1 refers to coumermycin-A1.

FIGS. 2A-2G depict the identification of Human CMG helicase small chemical Inhibitors (CMGi). (FIG. 2A) Purification of active human CMG helicase (hCMG) and steps involved in screening for hCMG inhibitors using orthogonal biochemical assays. (FIG. 2B) hCMG helicase assessed in primary ATPase assay to determine amounts of hCMG necessary for chemical library screening (10 μL reactions). The fluorescent-polarization (FP) window is determined using analytes from the screening assay without added enzyme. (FIG. 2C) FP ATPase assays measuring hCMG activity from a preparation of hCMG holo-helicase compared to a parallel preparation of hCMG lacking co-expression of Mcm4. The assay compared ~15 fmol/2 μL hCMG to the same amount of sample from hCMG(-Mcm4). (FIG. 2D) Small chemical inhibitors of hCMG ATPase activity were identified in primary screening at 1 mM chemical concentrations, repeated at 500 PM. The DMSO solvent was compared as a control. (FIG. 2E) Potential hCMG inhibitors identified in the primary assay assessed in a secondary fork-unwinding assay measuring effects on hCMG helicase activity. Clorobiocin (top panels) and coumermycin-A1 (CA1; lower panels) were compared to novobiocin. (FIG. 2F) Fork-unwinding assay with purified hCMG helicase determined the IC₅₀ for helicase inhibition by CA1. (FIG. 2G) FP ATPase assay measuring hCMG activity in presence of increasing [CA1]. Percent change was based on a comparison to an ADP-ATP standard curve.

FIGS. 3A-3F depict that CMGi inhibit CMG helicase activity using an ATP-competitive mechanism. (FIG. 3A) (Left) Structures of coumermycin-A1 (CA1), clorobiocin, and novobiocin. Orange arrows denote 5-methylpyrrole on the noviose sugar (purple boxes); blue open arrow, carbamate. (Right) Structures of synthetic compounds generated in this study. (FIG. 3B) The kinetics of hCMG helicase activity were determined +/−CA1 (2.5 μM) in increasing [ATP], shown using Michaelis-Menten and double-reciprocal plots. Amount of ssDNA separated from radio-labeled DNA forks per 30 minutes was quantified. CA1 competes with ATP to inhibit hCMG helicase activity, raising the Kₘ for [ATP]. (FIG. 3C) Structural image (top left) of the hCMG from available cryo-EM (PDB accession 6XTX). Inset boxes: regions enlarged in later panels showing CA1 docked in channels/ATPase clefts. White arrows: direction of CAI insertion. Top right (square panels), ATP bound in Mcm3-Mcm7 ATPase cleft and CA1 docked in the channel leading to Mcm3-Mcm7 ATPase cleft. Bottom left, side view of CA1 docked in Mcm3-Mcm7. Bottom right, CA1 in Mcm3-Mcm7 partially overlapping position where ATP binds. (FIG. 3D) CA1 docked in the channels leading to Mcm4-Mcm6 or Mcm5-Mcm3 ATPase clefts. Docking was performed using Autodock and Pymol software; predicted binding energies for CA1 shown on each panel. (FIG. 3E) hCMG helicase assays performed with indicated compounds. (FIG. 3F) hCMG helicase assays used to determine IC₅₀ for MBC.

FIGS. 4A-4G depict that CMGi inhibit Mcm2-7 ATPase-dependent MCM loading and CMG assembly. (FIG. 4A) Cell viability (Titer-Glo) assays using HaCaT cells. Based on IC₅₀ determination here for CA1, 15 μM of CA1 or novobiocin was used in the next experiments. (FIG. 4B) HaCaT cells synchronized and released into G1 (time 0 hr) were assessed for DNA replication after exposure to compounds at time of release. BrdU-labeled cells were assessed at times indicated using immunofluorescence (IF) methods and three fields were averaged per condition, ±1 s.d. Note for HaCaT experiments in remainder of figures that 15 hrs is G1/S, and 18 hrs is early S-phase. (FIG. 4C) Experimental design for FIGS. D-G. (FIG. 4D) Immunoblots of chromatin-bound or total proteins from synchronized HaCaT cells treated with compounds at time of G1 release (0 hr), and assessed at 18 hrs. (FIG. 4E) Immunoblots of chromatin-bound or total proteins from synchronized HaCaT cells treated with compounds at 6 hr (middle GI), and assessed at times indicated. (FIG. 4F) Synchronized HaCaT cells treated with compounds or DMSO in late G1 (12 hrs) and assessed for DNA replication using BrdU labeling at 18 hrs. Three fields of cells were averaged per condition, ±1 s.d. (FIG. 4G) Immunoblots of chromatin-bound or total proteins from synchronized HaCaT cells treated with compounds at 12 hr (late G1), and assessed at times indicated.

FIGS. 5A-5B depict that CMGi do not affect kinases involved in CMG/MCM assembly in human cells. Immunoblots of total proteins from synchronized HaCaT cells treated with compounds at time of release (0 hr; beginning of G1 phase) and collected after 18 hrs of exposure. (FIG. 5A) DDK-dependent phosphorylation sites on Mcm2 were analyzed with phospho-specific antibodies. (FIG. 5B) Cdk1- and Cdk2-dependent targets (Rb, Cdc6, and PP1α) were analyzed with phospho-specific antibodies.

FIGS. 6A-6E depict that CMGi disrupt structural co-stability of the hCMG and replisome in S-phase. (FIG. 6A) Experimental design for panels B&C. (FIG. 6B) Synchronized HaCaT cells were released into the cell cycle and allowed to progress into early S-phase (18 hrs). Cells were then treated with compounds and analyzed 3 hrs later for DNA replication using BrdU labeling. Results are averages from three fields, 1 s.d. CA1/novobiocin, 15 μM; etoposide, 5 μM; these doses were also used in the following panels. (FIG. 6C) Immunoblots of chromatin-bound or total proteins from synchronized HaCaT cells treated with compounds at 18 hr (early S-phase) and assessed at times indicated. (FIG. 6D) Experimental design (left) for in vitro assessment of CA1 effects on hCMG complexes. A nuclear extract was prepared from synchronized HaCaT (20 hrs after release; middle S-phase) and subjected to immunoprecipitation with anti-Psf1, anti-Mcm2, or IgG control. Samples were separated in half, then treated with DMSO or CA1 (15 μM) for 30 minutes prior to immunoblotting for (co-)precipitated proteins. (FIG. 6E) Experimental design (left) for in vitro assessment of CA1 effects on hCMG and replisome complexes from asynchronous HEK-293T cells stably expressing ectopic Flag-Mcm2. A nuclear extract was prepared and subjected to immunoprecipitation with anti-Flag or IgG as a control. Samples were treated with DMSO or CA1 (15 μM) for 30 minutes and immunoblotted for co-precipitated proteins (or Mcm2).

FIGS. 7A-7C depict that CMGi induce DNA damage and selectively reduce tumor cell viability. (FIG. 7A) Cell Titer Glo viability assays for CA1/CMGi sensitivity were performed on 143B (OS), Psn1 (PDAC), and H460 (NSCLC) tumor lines. Novobiocin was tested against 143B cells as a comparison. (FIG. 7B) Immunoblot of chromatin-bound proteins (as indicated) from asynchronous 143B OS cells treated with 10 μM novobiocin or increasing concentrations of CA1 for 24 hrs. (FIG. 7C) Immunoblot of total proteins from Psn1, H460, 143B, and HaCaT cells treated with 5 μM CA1/CMGi or DMSO/novobiocin controls, or HaCaT treated with 15 μM CA1/CMGi (right side). The Psn1 and H460 cells were treated for 24 hrs, and 143B and HaCaT cells were treated for 48 hrs. Probing of blots was done with anti-gamma-H2AX (DNA damage signal) or anti-cleaved-Parp (apoptotic indicator). Anti-GAPDH is a loading control.

FIGS. 8A-8C depict purity and ATPase-dependent activity of isolated hCMG Helicase. (FIG. 8A) Silver stain gel of a sample (5 μL) after the Flag elution step prior to loading onto the glycerol gradient (with added PreScission enzyme; *P). CMG subunits sometimes produce multiple bands (e.g., Cdc45), and a diffuse band labeled ‘b’ is present and likely represents modified Cdc45. These observations closely match the hCMG purity and banding patterns obtained in previous reports. (FIG. 8B) High molecular weight fractions after glycerol gradient enrichment were assessed by immunoblotting for presence of all hCMG subunits in the 750-800 kDa range (based on thyroglobulin location). (FIG. 8C) Fork-unwinding assays performed using the purified hCMG (~15 fmol/20 μL reaction) were used to verify that ATP hydrolysis is required for strand displacement (helicase) activity by the hCMG enzyme. Where ATP was added, 500 μM [ATP] was used. Slowly-hydrolyzable ATP-gamma-S was added in increasing concentrations, as indicated. Phosphorimager analysis of the resultant gel is shown in the graph, with the two adjacent gel images originating from the same gel/experiment.

FIGS. 9A-9C depict calibration and optimization of ATPase Transcreener ADP² assay conditions. (FIG. 9A) ATP can bind to some extent to the patented monoclonal ADP² Antibody used in the ADP-sensing assays, but ADP has a much higher binding affinity. Since ATP is used at high 500 μM concentrations in the primary ADP-sensing (ATPase) assay, adjusting the amount of ADP-fluor:Antibody ratio was needed to achieve proper read-outs in the ~EC80-85 range of the assay, according to manufacturer (Bellbrook Labs). Titrations were performed with increasing ADP² Antibody levels, 500 μM ATP, and 4 nM ADP-fluor Tracer to determine a suitable ratio of Tracer:Ab to use to offset the higher ATP levels that are present. (FIG. 9B) A standard curve assessing the ability of the primary fluorescent-polarization (FP)-based ADP-sensing assay to detect low levels of ADP produced in the 500 μM ATP starting concentration. The Z′ factor for each ratio of ADP:ATP was calculated according to manufacturer instructions. The system can detect very small ADP levels produced, as low as 1-3% changes at acceptable Z′ levels that produce at least 50% changes in the FP read-outs from the plate reader. (FIG. 9C) ATP-gamma-S cannot be used as a positive control for hCMG inhibitor screening with the Transcreener ADP² Assay, since it competes with the ADP² Antibody used in the analysis. Note FP reading decreases with increasing [ATP-gamma-S], which decreases the mP window and renders hCMG measurements unreliable.

FIGS. 10A-10D depict that hCMG helicase is not sensitive to quinolones, and human Topo-II and related hexameric helicases are sensitive to CA1 at high concentrations. (FIG. 10A) Fork-unwinding assays performed using purified hCMG enzyme +/−100 μM or 1000 μM concentrations of the indicated quinolone compounds. Quantitation below the gel (on left) indicates measurements obtained from PhosphorImager analysis of radio-labeled ssDNA bands. (FIG. 10B) In vitro decatenation assay demonstrates that CA1 has no inhibitory effect on human Topoisomerase-II at low concentrations used in this report in cell-based experiments. Novobiocin also has no effect at 1 mM. (Right side) Fork-unwinding (helicase) assay demonstrating that etoposide does not inhibit hCMG activity. (FIG. 10C) SV40-TAg helicase assays and CA1 IC₅₀ determinations. (FIG. 10D) HPV18-E1 and HPV16-E1 helicase assays using purified helicase domains (HD). The CA1 IC₅₀ was determined for HPV16-E1, graph on right.

FIG. 11 depicts that CMGi compete with high ATP concentrations. hCMG helicase assays were performed in the presence of 500 μM or 2 mM [ATP] to determine IC₅₀ values for CA1 under higher ATP levels that exist in cells. Results from assays are plotted together in the graph showing that the IC₅₀ for CA1 is similar under both ATP conditions. However, note that in the helicase gel photo higher [ATP] produces higher relative hCMG activity at all tested CA1 concentrations due to competition between ATP and CA1.

FIGS. 12A-12F depict docking of the CMGi clorobiocin in hCMG ATPase channels. (FIG. 12A) Structural image of the hCMG holo-enzyme from publicly available cryo-EM data (PDB accession code 6XTX). This is the same image used in FIG. 3C, as a reference. Inset boxes indicate regions that are enlarged in the following panels where clorobiocin is docked, for clarity here showing the larger CA1 molecule docked into channels. The white arrow on each box denotes the direction of the clorobiocin/CA1 insertion into the channels. (FIGS. 12B, 12C, and 12D) Clorobiocin docked in the channel leading to the Mcm4-Mcm6 ATPase cleft, the Mcm3-Mcm7 ATPase cleft, or the Mcm5-Mcm3 ATPase cleft, respectively. The noviose-sugar head group of clorobiocin (purple boxes in FIG. 3A) is inserted into the ATPase clefts, while the benzamide ‘tail’ of clorobiocin extends outside of the channels. Docking was performed using Autodock and Pymol software, with predicted binding energies for clorobiocin calculated by software shown on each panel. (FIG. 12E) Structural images of the three MCM ATPase sites that are accessible for and can dock CA1 and clorobiocin. Each ATPase cleft is shown with bound nucleotide (magenta color) based on cryo-EM data. (FIG. 12F) Images of the three MCM ATPase sites that are not accessible for CA1 or clorobiocin. Bound nucleotide is shown in two sites based on cryo-EM data, while third is in Apo state. Note that the views are at the same scale and zoom level as those in this figure, and all show protrusions or small channels physically restrictive to chemical binding.

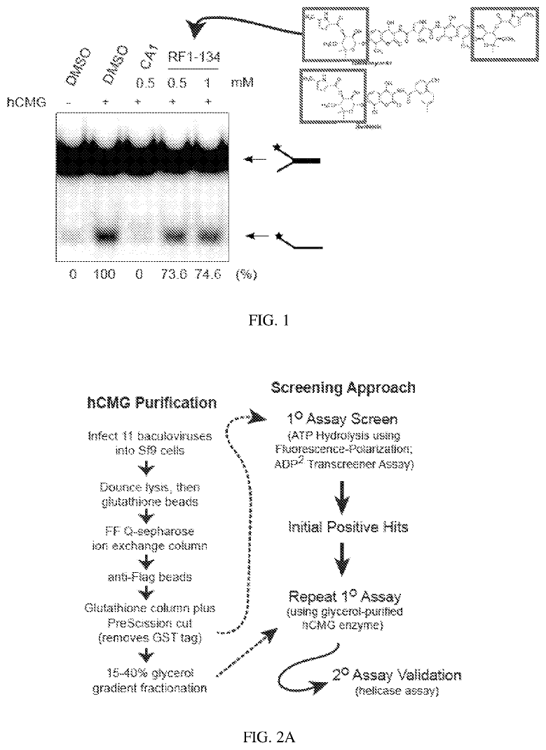

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an,” and “the” include plural referents unless the context clearly dictates otherwise.

It should be noted that ratios, concentrations, amounts, and other numerical data can be expressed herein in a range format. It can be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y’, and ‘about z’ as well as the ranges of ‘less than x,’ less than y’, and ‘less than z’. Likewise, the phrase ‘about x, y, z, or greater’ should be interpreted to include the specific ranges of ‘about x,’ ‘about y’, and ‘about z’ as well as the ranges of ‘greater than x,’ greater than y’, and ‘greater than z.’ In addition, the phrase “about ‘x’ to ‘y’,” where ‘x’ and ‘y’ are numerical values, includes “about ‘x’ to about ‘y’.”

It is to be understood that such a range format is used for convenience and brevity and, thus, should be interpreted in a flexible manner to include not only the numerical values explicitly recited as the limits of the range but also all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter, or other quantity or characteristic is “about,” “approximate,” or “at or about” whether or not expressly stated to be such. It is understood that where “about,” “approximate,” or “at or about” is used before a quantitative value, the parameter also includes the specific quantitative value itself unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition can also be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single-dose compositions can contain such amounts or submultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons, or virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied, for example, by increasing or decreasing the amount of a disclosed compound and/or pharmaceutical composition, changing the disclosed compound and/or pharmaceutical composition administered, changing the route of administration, changing the dosage timing, and so on. Dosage can vary and can be administered in one or more doses daily for one or several days. Guidance can be found in the literature for appropriate dosages for given classes of pharmaceutical products.

As used herein, the terms “optional” or “optionally” mean that the subsequently described event or circumstance can or cannot occur and that the description includes instances where said event or circumstance occurs and instances where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to humans and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as a cancer. The effect can be therapeutic in terms of a partial or complete cure of a disease, condition, symptom, or adverse effect attributed to the disease, disorder, or condition. The term “treatment” as used herein can include any treatment of a disorder in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment,” as used herein, can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating” can include inhibiting the disease, disorder, or condition, e.g., impeding its progress, and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect or to decreasing the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R-) or (S-) configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Compounds described herein may contain one or more double bonds and, thus, potentially give rise to cis/trans (E/Z) isomers, as well as other conformational isomers.

Unless stated to the contrary, all such possible isomers are contemplated, as well as mixtures of such isomers.

Compounds described herein may also present as an equilibrium of tautomers. For example, ketones with an α-hydrogen can exist in an equilibrium of the keto form and the enol form. Likewise, amides with an N-hydrogen can exist in an equilibrium of the amide form and the imidic acid form. Unless stated to the contrary, all possible tautomers of the compounds described herein are contemplated.

A dash (“—”) that is not between two letters or symbols is used to indicate a point of attachment for a substituent. For example, —(C═O)NH₂ is attached through the carbon of the keto (C═O) group.

As used herein, the symbol

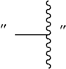

(which hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

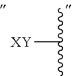

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

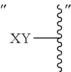

infers that when R³ is “XY,” the point of attachment bond is the same bond as the bond by which R³ is depicted as being bonded to CH₃.

The term “substituted,” as used herein, means that any one or more hydrogens on the designated atom or group are replaced with a moiety selected from the indicated group, provided that the designated atom's normal valence is not exceeded and the resulting compound is stable. For example, when the substituent is oxo (i.e., ═O), two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react, or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person in the context in which said functional groups are recited.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one aspect, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one aspect, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one aspect, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing I to 2 sulfur atoms and I to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring that contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, such as groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one aspect, the total number of S and O atoms in the heteroaryl group is not more than 2. In another aspect, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like) or by reacting free base forms of these compounds with a stoichiometric amount of the appropriate acid. Such reactions are typically carried out in water, an organic solvent, or a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts that are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, the term “derivative” refers to a compound having a structure derived from the structure of a parent compound (e.g., a compound disclosed herein) and whose structure is sufficiently similar to those disclosed herein and based upon that similarity would be expected by one skilled in the art to exhibit the same or similar activities and utilities as the claimed compounds, or to induce, as a precursor, the same or similar activities and utilities as the claimed compound. Exemplary derivatives include but are not limited to, salts, esters, amides, salts of esters or amides, and N-oxides of a parent compound.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Sigma-Aldrich (formally MilliporeSigma, Burlington, MA) or Thermo Fisher Scientific Inc. (Waltham, MA) or are prepared by methods known to those skilled in the art following procedures set forth in references such as Fieser and Fieser's Reagents for Organic Synthesis (John Wiley and Sons, 2007); Organic Reactions (John Wiley and Sons, 2004); March's Advanced Organic Chemistry, (John Wiley and Sons, 8^{th} Edition); and Larock's Comprehensive Organic Transformations (John Wiley and Sons, 3^{rd} edition, 2017).

### Compounds

The present disclosure provides compounds that may be useful as degraders of helicases, such as SF3 and/or SF6 helicases, and more particularly, for example, CMG helicase and HPV E1 helicase. The present compounds have utility in the treatment of medical disorders mediated by an SF3 and/or an SF6 helicase, such as cancer.

In one aspect, a compound is provided of Formula I:

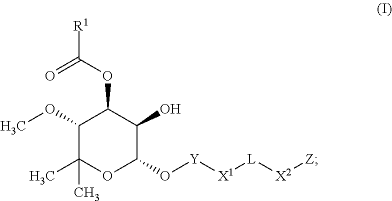

or a pharmaceutically acceptable salt thereof;
wherein:
R¹ is independently selected at each occurrence from monocyclic or bicyclic heteroaryl and monocyclic or bicyclic heterocycloalkyl, wherein each R¹ optionally includes at least one ring nitrogen atom substituted with R⁷ as allowed by valency, and wherein each R¹ is optionally substituted with 1, 2, 3, or 4 groups selected from R⁸ as allowed by valency;
Y is selected from a bond, bicyclic aryl, or bicyclic heteroaryl, wherein Y is optionally substituted with 1, 2, 3, or 4 groups independently selected from R² as allowed by valency;
X¹ is selected from a bond or —NR^{a}—;
L is selected from a bond or -L¹-L²-L³-L⁴-L⁵-L⁶-L⁷-;
L¹, L², L³, L⁴, L⁵, L⁶, and L⁷ are independently selected from:
a) a bond;
b) —C(═O)—;
c) —C≡C—;
d) —NR^{a}—;
e) —O—;
d) C₁-C₁₀ alkyl;
e) cycloalkyl optionally substituted with 1, 2, 3, or 4 substituents independently selected from R³;
f) heterocycloalkyl optionally substituted with 1, 2, 3, or 4 substituents independently selected from R⁴;
g) aryl optionally substituted with 1, 2, 3, or 4 groups independently selected from R⁵;
h) heteroaryl optionally substituted with 1, 2, 3, or 4 groups independently selected from R⁶;
i) —NR^{a}(C═O)—;
j) —C(═O)NR^{a}—;
k) —C(═O)(C₁-C₆ alkyl)-;
l) —(C₁-C₆ alkyl)C(═O)—;
m) —(C₁-C₆ alkyl)NR^{a}—;
n) —NR^{a}(C₁-C₆ alkyl)-;
o) —(C₁-C₆ alkyl)O—; and
p) —O(C₁-C₆ alkyl)-;
X² is selected from a bond, —NR^{a}—, —O—, —C≡C—, —C(═O)—, —S(═O)₂—, —C(═O)NR^{a}—, —NR^{a}C(═O)—, —S(═O)₂NR^{a}—, —NR^{a}S(═O)₂—, and —NR^{a}(C═O)NR^{b}—;
Z is an E3 ubiquitin ligase binding moiety;
R² is selected at each occurrence from halogen, hydroxy, nitro, —NHR^{a}, C₁-C₆ alkyl, C₁-C₆ haloalkyl, aryl, and oxo;
R³ and R⁴ are independently selected at each occurrence from halogen, hydroxy, —NHR^{a}, C₁-C₆ alkyl, C₁-C₆ haloalkyl and oxo;
R⁵ and R⁶ are independently selected at each occurrence from halogen, hydroxy, nitro, —NHR^{a}, C₁-C₆ alkyl, C₁-C₆ haloalkyl, and aryl;
R⁷ is selected from: hydrogen; C₁-C₄ alkyl; C₂-C₆ alkenyl; C₂-C₆ alkynyl; C₃-C₆ cycloalkyl; C₁-C₆ alkoxy; and (C₀-C₆ alkyl)(aryl) which is optionally substituted with halogen, C₁-C₆ alkyl, or C₁-C₆ alkoxy;
R⁸ is selected from: hydrogen; halogen; hydroxy; —NR^{a}R^{b}; nitro; C₁-C₆ alkyl; C₁-C₆ haloalkyl; C₃-C₆ cycloalkyl; C₃-C₆ heterocycloalkyl; C₂-C₆ alkenyl; C₂-C₆ alkynyl; C₁-C₆ alkoxy; (C₀-C₆ alkyl)(aryl) which is optionally substituted with halogen, C₁-C₆ alkyl, or C₁-C₆ alkoxy; and (C₀-C₆ alkyl)(heteroaryl) which is optionally substituted with halogen, C₁-C₆ alkyl, or C₁-C₆ alkoxy; and
R^{a} and R^{b} are independently selected at each occurrence from hydrogen and C₁-C₆ alkyl.

In some aspects of Formula I, R¹ is

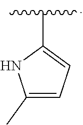 [SMILES: *c1ccc(C)[nH]1]

In some aspects of Formula I, R¹ is selected from:

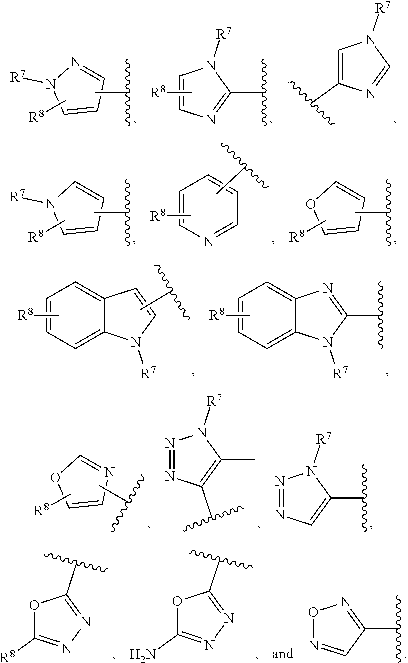

In some aspects of Formula I, R¹ is selected from:

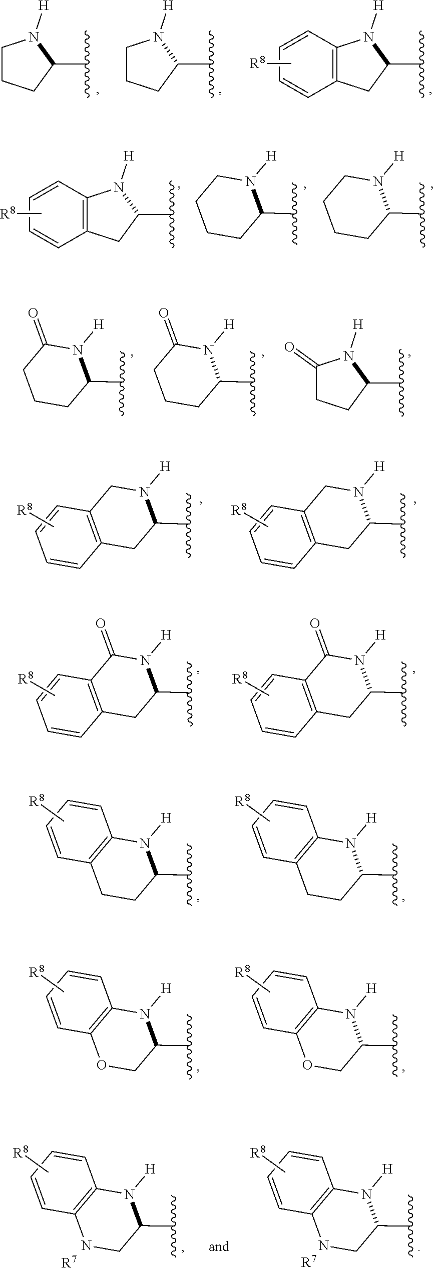

In some aspects of Formula I, Y is a bond.

In some aspects of Formula I, Y is

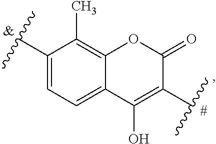 [SMILES: *c1ccc2c(O)c(*)c(=O)oc2c1C]

wherein & denotes the point of attachment to the neighboring oxygen atom and # denotes the point of attachment to X¹ and/or X².

In some aspects of Formula I, Y is selected from:

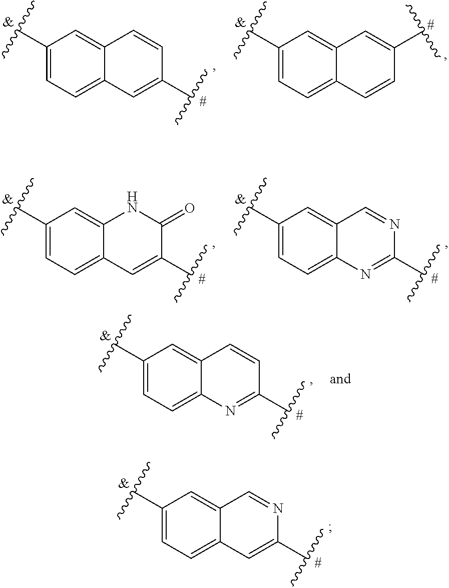 [SMILES: *c1ccc2cc(*)ccc2c1 ; *c1ccc2ccc(*)cc2c1 ; *c1ccc2cc(*)c(=O)[nH]c2c1 ; *c1ccc2nc(*)ncc2c1 ; *c1ccc2nc(*)ccc2c1 ; *c1ccc2cc(*)ncc2c1]

wherein & denotes the point of attachment to the neighboring oxygen atom and # denotes the point of attachment to X¹ and/or X².

In some aspects of Formula I, X¹ is —NH—.

In some aspects of Formula I, L¹, L², L³, L⁴, L⁵, L⁶, and L⁷ are each independently selected from: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)—; —O(CH₂)₆—;

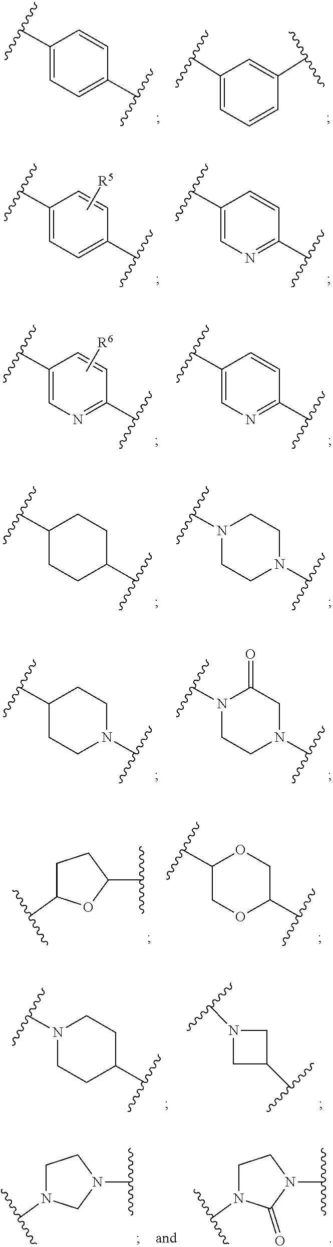

In some aspects of Formula I, L¹, L², L³, L⁴, L⁵, L⁶, and L⁷ are selected in such a way that: no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of L¹, L², L³, L⁴, L⁵, L⁶, and L⁷.

In some aspects of Formula I, L is selected from:

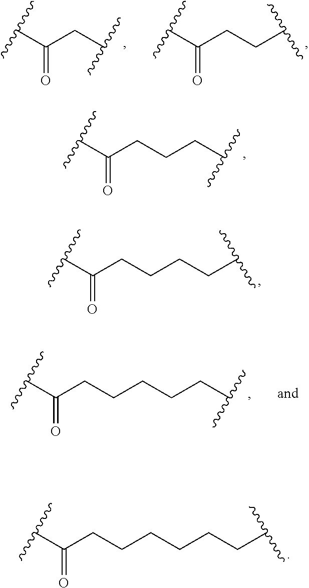 [SMILES: *CC(*)=O ; *CCC(*)=O ; *CCCC(*)=O ; *CCCCC(*)=O ; *CCCCCC(*)=O ; *CCCCCCC(*)=O]

In some aspects of Formula I, L is selected from:

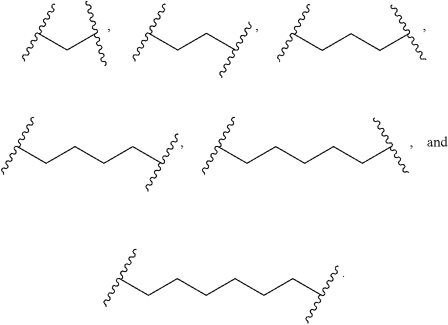

In some aspects of Formula I, L is selected from:

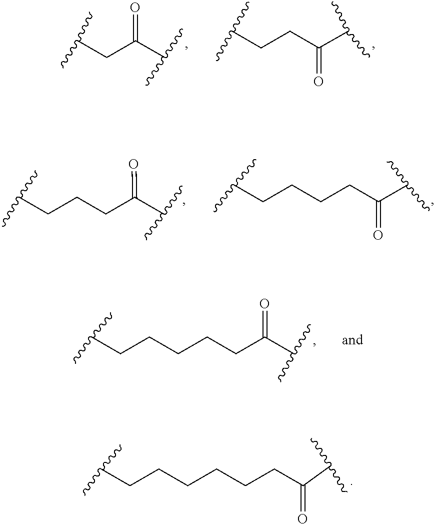 [SMILES: *CC(*)=O ; *CCC(*)=O ; *CCCC(*)=O ; *CCCCC(*)=O ; *CCCCCC(*)=O ; *CCCCCCC(*)=O]

In some aspects of Formula I, L is

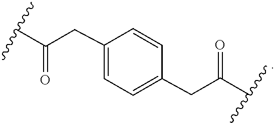 [SMILES: *C(=O)Cc1ccc(CC(*)=O)cc1]

In some aspects of Formula I, L is

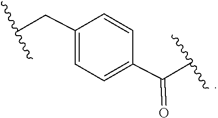 [SMILES: *Cc1ccc(C(*)=O)cc1]

In some aspects of Formula I, L is selected from:

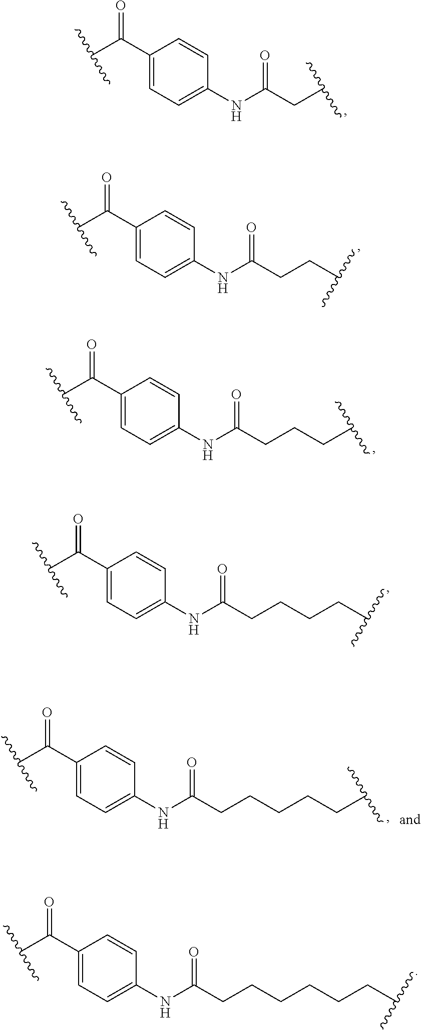 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

In some aspects of Formula I, L is selected from:

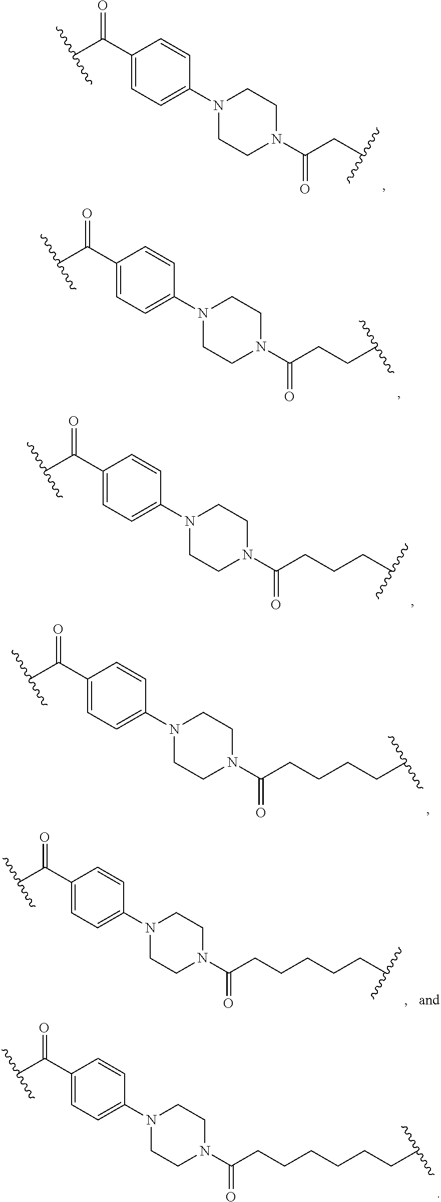 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

In some aspects of Formula I, L is selected from:

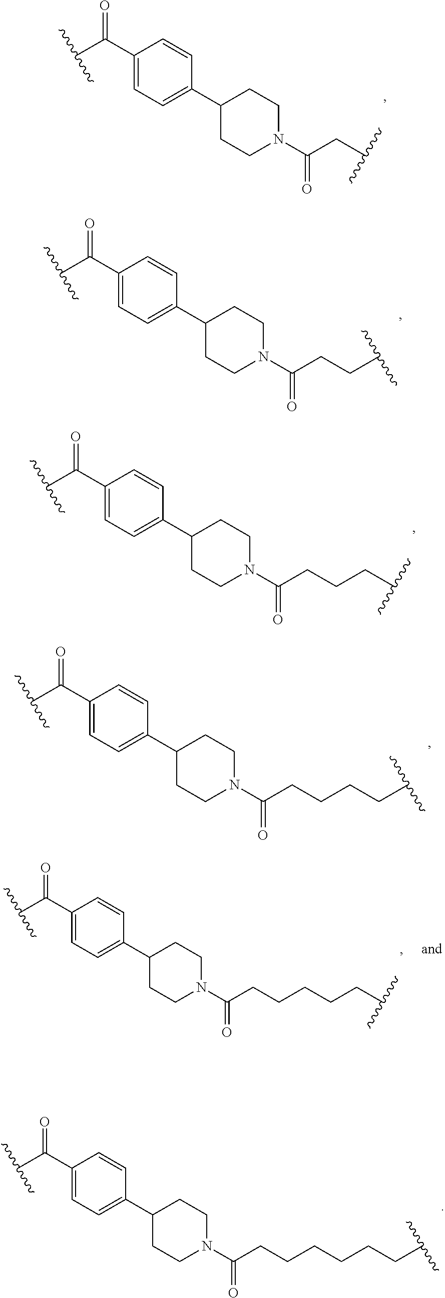 [SMILES: *CC(=O)N1CCC(c2ccc(C(*)=O)cc2)CC1 ; *CCC(=O)N1CCC(c2ccc(C(*)=O)cc2)CC1 ; *CCCC(=O)N1CCC(c2ccc(C(*)=O)cc2)CC1 ; *CCCCC(=O)N1CCC(c2ccc(C(*)=O)cc2)CC1 ; *CCCCCC(=O)N1CCC(c2ccc(C(*)=O)cc2)CC1 ; *CCCCCCC(=O)N1CCC(c2ccc(C(*)=O)cc2)CC1]

In some aspects of Formula I, L is selected from:

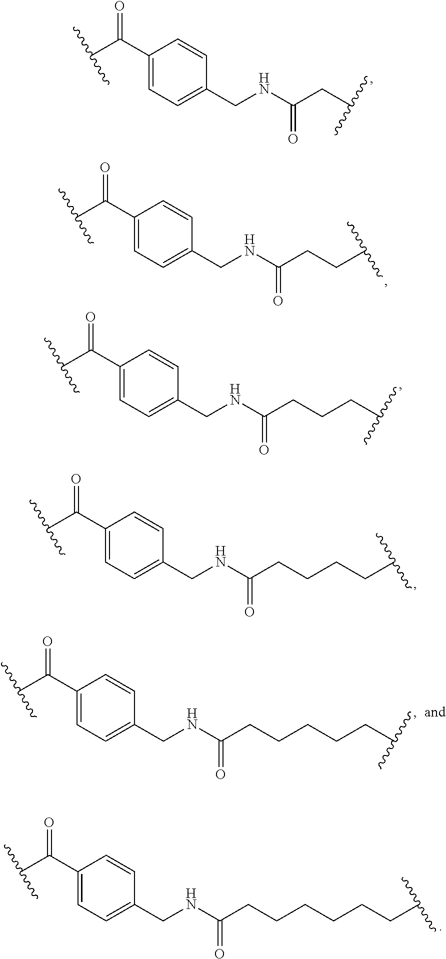 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

In some aspects of Formula I, L is selected from:

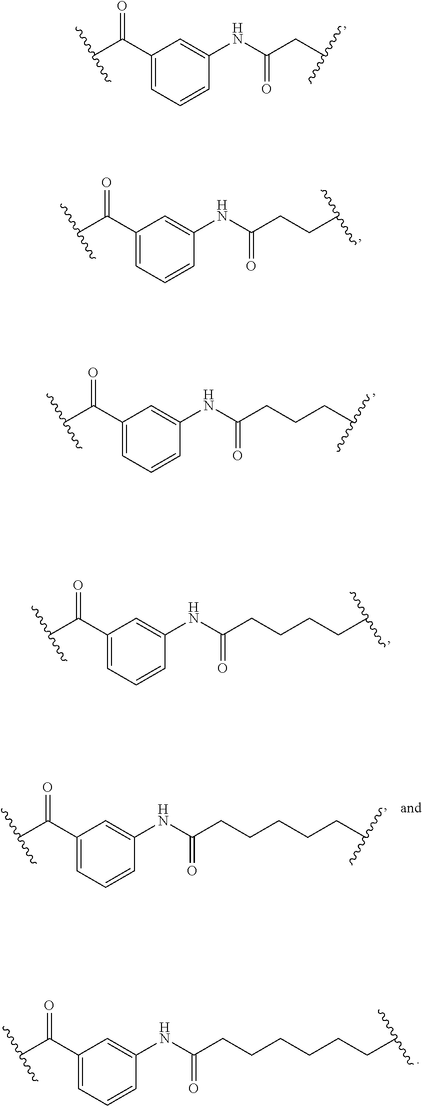 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

In some aspects of Formula I, L is selected from:

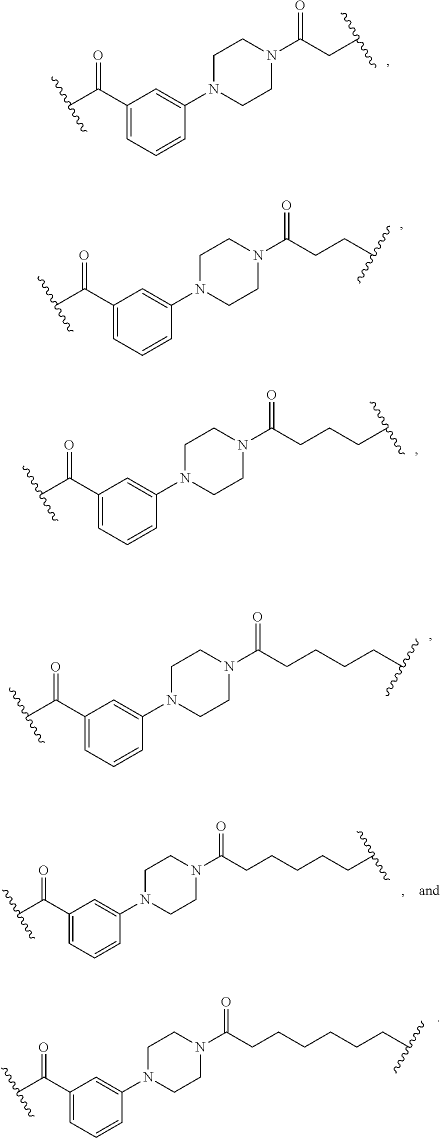 [SMILES: *CC(=O)N1CCN(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

In some aspects of Formula I, L is selected from:

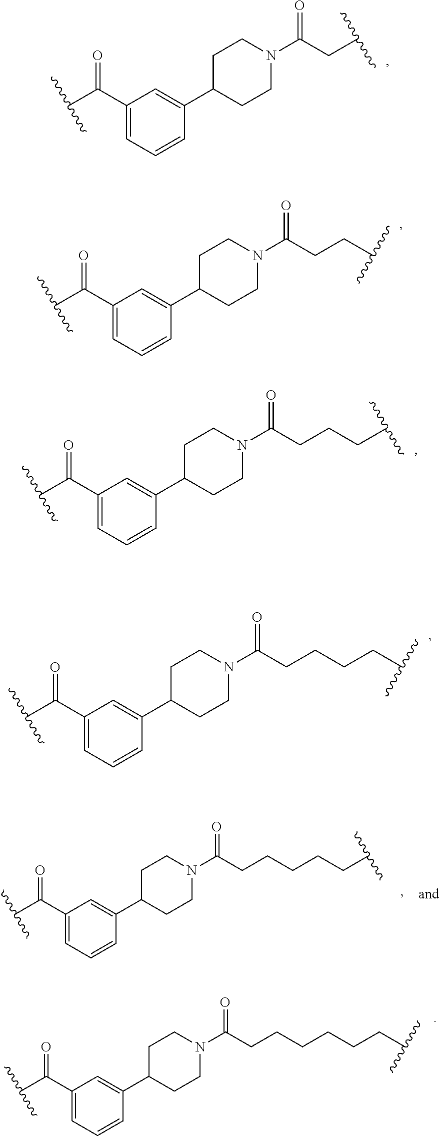 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

In some aspects of Formula I, L is selected from:

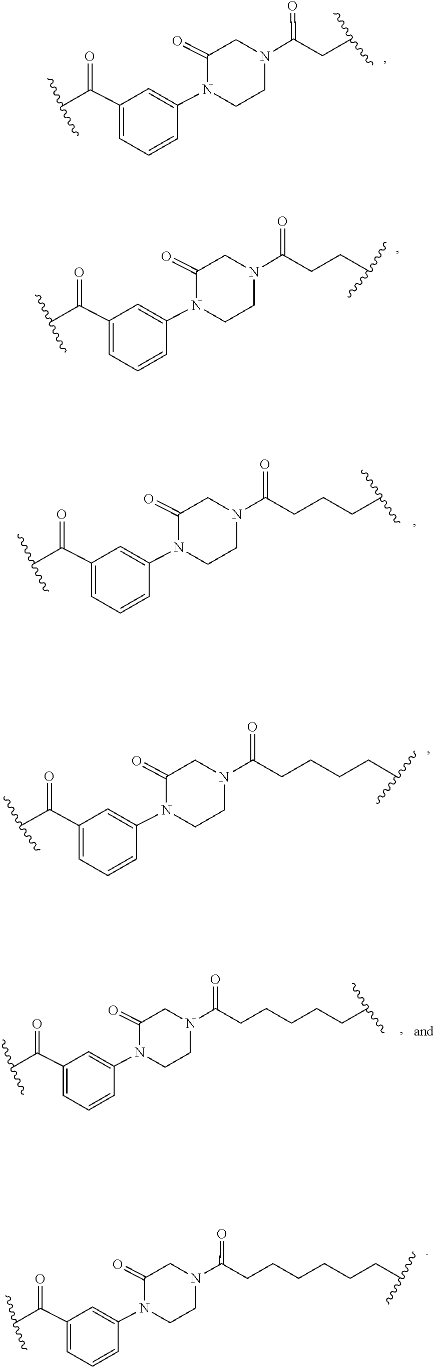 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

In some aspects of Formula I, L is selected from:

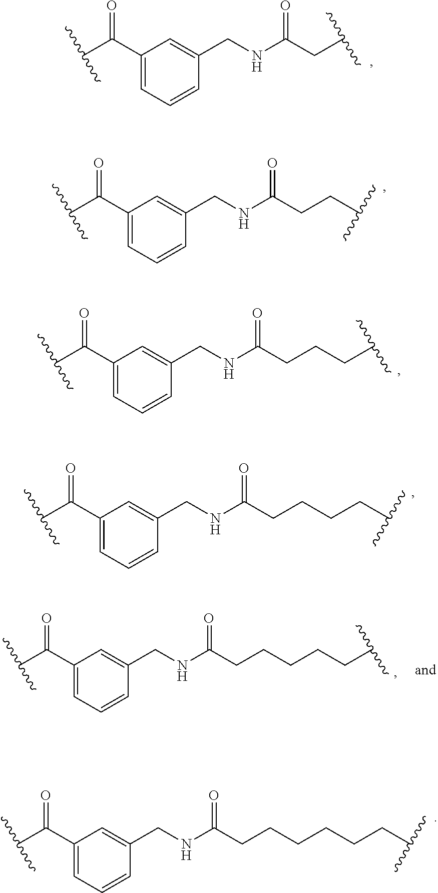 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCCC(=O)NCc1cccc(C(*)=O)c1 ; *CCCCCCC(=O)NCc1cccc(C(*)=O)c1]

In some aspects of Formula I, L is selected from:

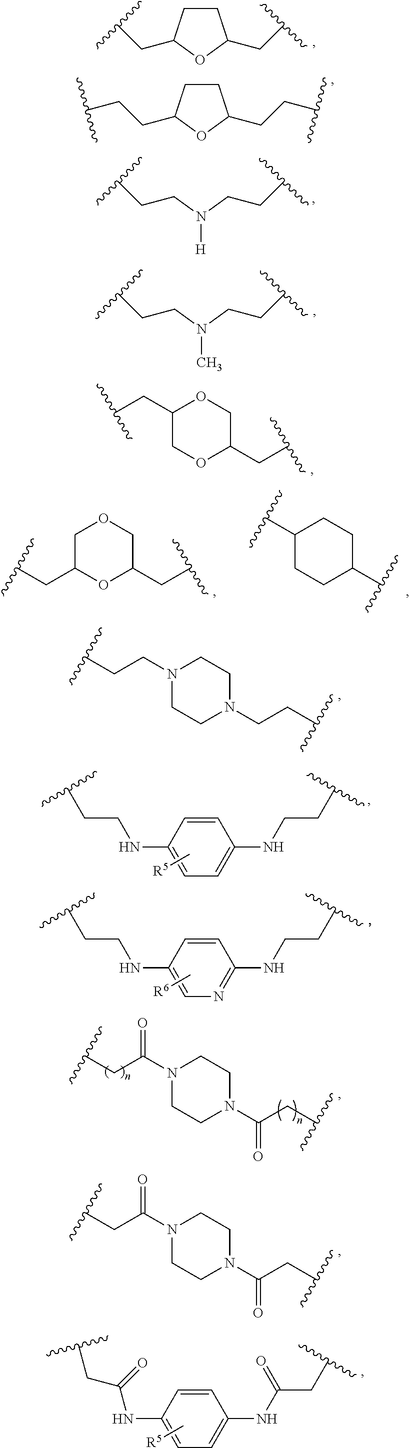

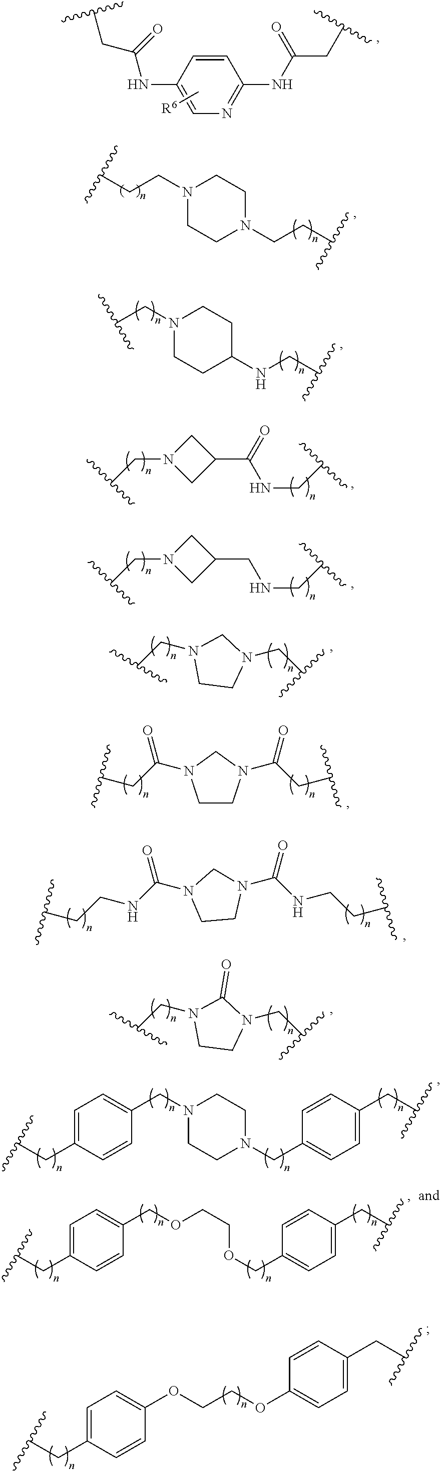

wherein n is independently selected at each occurrence from 1, 2, 3, 4, 5, and 6; and all other variables are as defined herein.

In some aspects of Formula I, X² is a bond. In some aspects of Formula I, X² is —NH—. In some aspects of Formula I, X² is —O—. In some aspects of Formula I, X² is —C≡C—. In some aspects of Formula I, X² is —C(═O)—. In some aspects of Formula I, X² is —S(═O)₂—. In some aspects of Formula I, X² is —C(═O)NH—. In some aspects of Formula I, X² is —NHC(═O)—. In some aspects of Formula I, X² is —S(═O)₂NH—. In some aspects of Formula I, X² is —NHS(═O)₂—. In some aspects of Formula I, X² is —NH(C═O)NH—.

In some aspects of Formula I, Z is selected from:

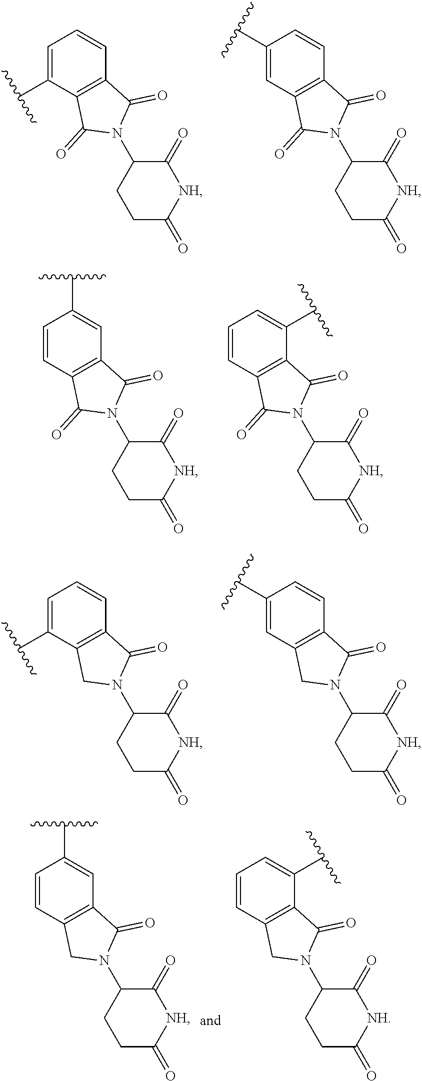

In some aspects of Formula I, Z is

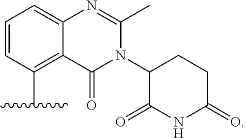 [SMILES: *c1cccc2nc(C)n(C3CCC(=O)NC3=O)c(=O)c12]

In some aspects of Formula I, Z is selected from:

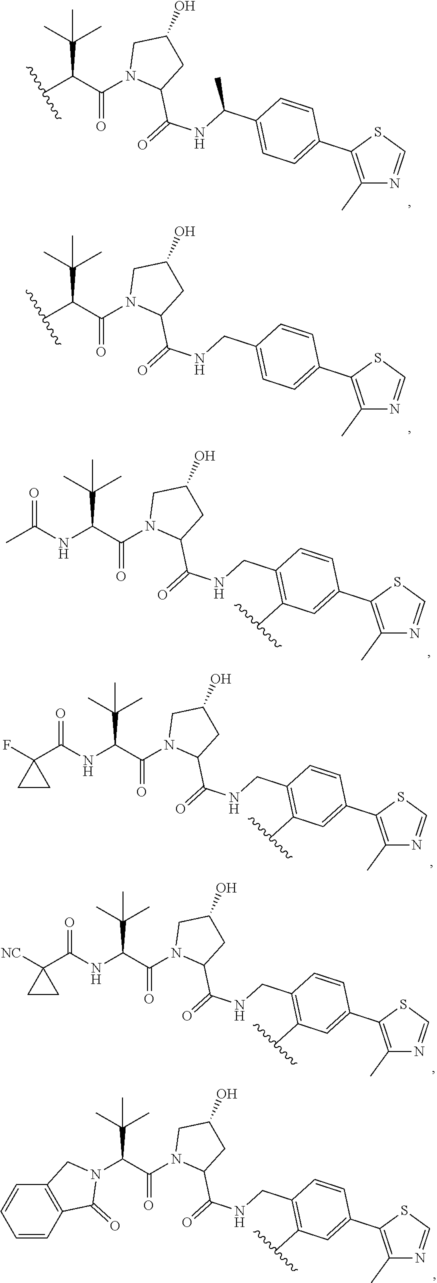 [SMILES: *[C@H](C(=O)N1C[C@H](O)CC1C(=O)N[C@@H](C)c1ccc(-c2scnc2C)cc1)C(C)(C)C ; *[C@H](C(=O)N1C[C@H](O)CC1C(=O)NCc1ccc(-c2scnc2C)cc1)C(C)(C)C ; *c1cc(-c2scnc2C)ccc1CNC(=O)C1C[C@@H](O)CN1C(=O)[C@@H](NC(C)=O)C(C)(C)C ; *c1cc(-c2scnc2C)ccc1CNC(=O)C1C[C@@H](O)CN1C(=O)[C@@H](NC(=O)C1(F)CC1)C(C)(C)C ; *c1cc(-c2scnc2C)ccc1CNC(=O)C1C[C@@H](O)CN1C(=O)[C@@H](NC(=O)C1(C#N)CC1)C(C)(C)C ; *c1cc(-c2scnc2C)ccc1CNC(=O)C1C[C@@H](O)CN1C(=O)[C@@H](N1Cc2ccccc2C1=O)C(C)(C)C]

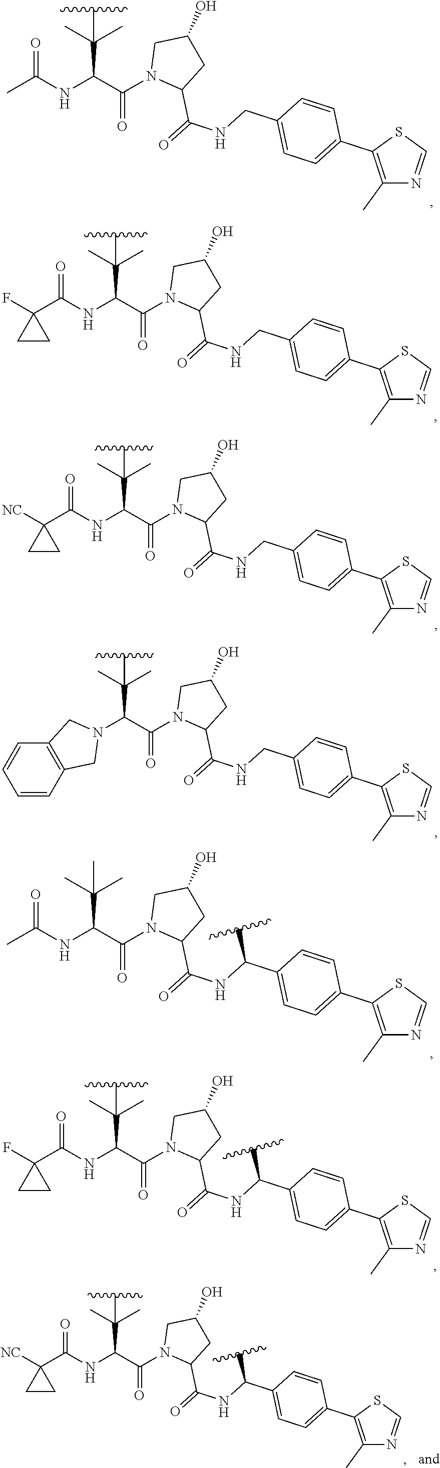

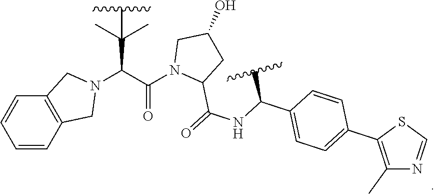 [SMILES: *[C@H](NC(=O)C1C[C@@H](O)CN1C(=O)[C@@H](N1Cc2ccccc2C1)C(*)(C)C)c1ccc(-c2scnc2C)cc1]

In some aspects of Formula I, Z is

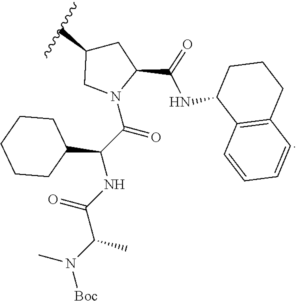 [SMILES: *[C@H]1C[C@@H](C(=O)N[C@@H]2CCCc3ccccc32)N(C(=O)[C@@H](NC(=O)[C@H](C)N(C)C(=O)OC(C)(C)C)C2CCCCC2)C1]

The present disclosure also includes compounds with at least one desired isotopic substitution of an atom at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P ³²P, ³⁵S, ³⁶Cl, and ¹²⁵I, respectively. In one aspect, isotopically labeled compounds can be used in metabolic studies (with ¹⁴C), reaction kinetic studies (with, for example, ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F-labeled compound may be particularly desirable for PET or SPECT studies. Isotopically labeled compounds of this disclosure and prodrugs thereof can generally be prepared by carrying out the procedures disclosed herein by substituting a readily available isotopically labeled reagent for a non-isotopically labeled reagent.

By way of general example and without limitation, isotopes of hydrogen, for example, deuterium (²H) and tritium (3H), may optionally be used anywhere in described structures that achieve the desired result. Alternatively, or in addition, isotopes of carbon, e.g., ¹³C and ¹⁴C, may be used. In one aspect, the isotopic substitution is replacing hydrogen with deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example, deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, enrichment, at any point, is above natural abundance and, in one aspect, is enough to alter a detectable property of the compounds as a drug in a human.

The compounds of the present disclosure may form a solvate with solvents (including water). Therefore, in one aspect, the disclosure includes a solvated form of the active compound. The term “solvate” refers to a molecular complex of a compound of the present disclosure (including a salt thereof) with one or more solvent molecules. Non-limiting examples of solvents are water, ethanol, dimethyl sulfoxide, acetone, and other common organic solvents. The term “hydrate” refers to a molecular complex comprising a disclosed compound and water. Pharmaceutically acceptable solvates in accordance with the disclosure include those wherein the solvent of crystallization may be isotopically substituted, e.g., D₂O, d₆-acetone, or d₆-DMSO. A solvate can be in a liquid or solid form.

A “prodrug,” as used herein, means a compound which, when administered to a host in vivo, is converted into a parent drug. As used herein, the term “parent drug” means any of the presently described compounds herein. Prodrugs can be used to achieve any desired effect, including to enhance the properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain aspects, the prodrug renders the parent compound more lipophilic. In certain aspects, a prodrug can be provided that has several prodrug moieties in a linear, branched, or cyclic manner. For example, non-limiting aspects include the use of a divalent linker moiety such as a dicarboxylic acid, amino acid, diamine, hydroxycarboxylic acid, hydroxyamine, di-hydroxy compound, or another compound that has at least two functional groups that can link the parent compound with another prodrug moiety and is typically biodegradable in vivo. In some aspects, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some aspects, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched, or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art, including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort, may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion), and/or various types of wetting agents. As used herein, the term “carrier” encompasses, but is not limited to, any excipient, diluent, filler, salt, buffer, stabilizer, solubilizer, lipid, stabilizer, or other material well-known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants, and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an anti-oxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, such as injectable aqueous or oleaginous suspensions, may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P., and isotonic sodium chloride solution. In addition, sterile, fixed oils are conventionally employed as a solvent or suspending medium. Any bland fixed oil can be employed, including synthetic mono- or diglycerides. In addition, fatty acids such as oleic acid are used in the preparation of injectables. In certain aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter or by incorporating sterilizing agents in the form of sterile solid compositions, which can be dissolved or dispersed in sterile water or other sterile injectable media prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate-controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors, including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some aspects, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors, including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity and in vivo activity in animal models. Methods for the extrapolation of effective dosages in mice and other animals to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the patient and can be determined by one of skill in the art. The dosage can be adjusted by the individual physician in the event of any counterindications. Dosage can vary and can be administered in one or more doses daily for one or several days.

### Methods of Use

The present disclosure also provides methods for treating or preventing cancer in a subject, comprising administering to the subject a therapeutically effective amount of a compound or composition disclosed herein. The methods can further comprise administering one or more additional therapeutic agents, such as anti-cancer agents or anti-inflammatory agents. Additionally, the method can further comprise administering a therapeutically effective amount of ionizing radiation to the subject.

Methods of killing a cancer or tumor cell are also provided comprising contacting the cancer or tumor cell with an effective amount of a compound or composition as described herein. In some aspects, the compounds induce degradation of CMG helicase. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow, pig, or horse, or other animals having an oncological disorder. In some aspects, the subject can receive the therapeutic compositions prior to, during, or after surgical intervention to remove part or all of a tumor.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compounds or compositions disclosed herein may comprise carcinomas, sarcomas, lymphomas, leukemias, germ cell tumors, or blastomas.

Carcinomas which may be treated by the compounds or compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compounds or compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compounds or compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstrom macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compounds or compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compounds or compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compounds compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent, or an assimilable edible carrier for oral delivery. In addition, the active compound can be incorporated into sustained release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab. These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

In another aspect, methods are provided for the treatment of medical disorders associated with a helicase, for example an SF3 and/or SF6 helicase, by administering a compound of Formula I, or pharmaceutically acceptable salts thereof. In particular aspects, the compounds described herein may be used in the treatment of cancer, either alone or in combination with one or more additional therapeutic agents, for example a chemotherapeutic agent. In some aspects, the helicase comprises CMG helicase. In other aspects, the helicase comprises HPV E1 helicase.

Thus in one aspect, a method is provided for treating a cancer in a subject in need thereof, the method comprising administering a therapeutically effective amount of a compound of Formula I, or a pharmaceutically acceptable salt thereof. In some aspects, the compound of Formula I, or a pharmaceutically acceptable salt thereof, is administered as a pharmaceutical composition as further described herein. In some aspects, the subject is a human. In some aspects, the cancer is associated with dysregulation of a helicase, for example an SF3 and/or SF6 helicase. In some aspects, the cancer is associated with CMG helicase. In some aspects, the cancer is associated with HPV E1 helicase.

In another aspect, a method is provided for treating cancers associated with elevated expression levels of Myc and/or elevated expression levels of Cyclin E. Elevated levels of Myc and Cyclin E have been associated overactivation of CMG helicases, leading to diminished reserve MCMs available to allow the cancer cell to successfully complete the S-phase of the cell cycle. Upon exposure of the cancer cell to a CMG helicase inhibitor such as those described herein, the cancer cell faces diminished survival and potentially cell death.

Thus, in one aspect, a method is provided for treating a cancer in a subject in need thereof, the method comprising:
(a) determining whether the cancer is characterized by elevated Myc expression and/or elevated Cyclin E expression; and
(b) if the cancer is determined to be characterized by elevated Myc expression and/or elevated Cyclin E expression in (a), administering a therapeutically effective amount of a compound of Formula I, or a pharmaceutically acceptable salt thereof, either alone or in combination with one or more additional therapeutic agents (such as a chemotherapeutic or cytotoxic agent).

In another aspect, a method of treating a cancer associated with elevated Myc expression and/or elevated Cyclin E expression is provided comprising administering a therapeutically effective amount of a compound of Formula I, or a pharmaceutically acceptable salt thereof, either alone or in combination with one or more additional therapeutic agents (such as a chemotherapeutic or cytotoxic agent).

In yet another aspect, a method is provided for promoting degradation of CMG helicase in a eukaryotic cell comprising contacting the cell with an effective amount of a compound of Formula I, or a pharmaceutically acceptable salt thereof, as described herein. In some aspects, the eukaryotic cell is a human cell.

In yet another aspect, a method for treating cancer in a subject in need thereof is provided, the method comprising:
(a) determining whether the cancer is associated with one or more signs of replicative stress; and
(b) if the cancer is determined to be associated with one or more signs of replicative stress, administering a therapeutically effective amount of a compound of Formula I, or a pharmaceutically acceptable salt thereof.

In another aspect, a method is provided for treating cancer in a subject in need thereof, wherein the cancer has been previously determined to be associated with one or more signs of replicative stress, the method comprising administering a therapeutically effective amount of a compound of Formula I, or a pharmaceutically acceptable salt thereof.

In some aspects, the one or more signs of replicative stress may comprise Myc overexpression, CyclinE overexpression, Rb loss, p53 loss, PolQ overexpression, or combinations thereof. In some aspects, the one or more signs or replicative stress comprises Myc overexpression. In some aspects, the one or more signs or replicative stress comprises CyclinE overexpression. In some aspects, the one or more signs or replicative stress comprises Rb loss. In some aspects, the one or more signs or replicative stress comprises p53 loss. In some aspects, the one or more signs or replicative stress comprises PolQ overexpression.

In yet another aspect, a method for treating cancer in a subject in need thereof is provided, the method comprising:
(a) determining whether the cancer harbors one or more inherited or acquired germ-line mutations; and
(b) if the cancer is determined to harbor one or more inherited or acquired germ-line mutations in (a), administering a therapeutically effective amount of a compound of Formula I, or a pharmaceutically acceptable salt thereof.

In another aspect, a method for treating cancer in a subject in need thereof is provided, wherein the cancer has been previously determined to harbor one or more inherited or acquired germ-line mutations, the method comprising administering a therapeutically effective amount of a compound of Formula I, or a pharmaceutically acceptable salt thereof.

In some aspects, the one or more inherited or acquired germ-line mutations may comprise loss of: p53, Rb, BRCA1, BRCA2, ATM, a xeroderma pigmentosum gene (such as XPA, XPB, XPC, XPD, XPE, XPF, or XPG), a mismatch repair gene (such as MSH2, MLH1, MSH6, PMS2), WRN, BLM, a Fanconi anemia gene (such as FANCA, FANCB, FANCC, FANCD2, FANCE, FANCF, FANCG, FANCI, FANCJ, FANCL, FANCM, FANCN, FANCO, FANCP, FANCQ, FANCT, FANCU, FANCV, or FANCW), NBS, Chek2, RecqL4, MYH, PALB2, BACH1, RAC51C, or combinations thereof.

In some aspects, the one or more inherited or acquired germ-line mutations comprise loss of p53. In some aspects, the one or more inherited or acquired germ-line mutations comprise loss of Rb. In some aspects, the one or more inherited or acquired germ-line mutations comprise loss of BRCA1. In some aspects, the one or more inherited or acquired germ-line mutations comprise loss of BRCA2. In some aspects, the one or more inherited or acquired germ-line mutations comprise loss of ATM. In some aspects, the one or more inherited or acquired germ-line mutations comprise loss of XPA. In some aspects, the one or more inherited or acquired germ-line mutations comprise loss of XPB. In some aspects, the one or more inherited or acquired germ-line mutations comprise loss of XPC. In some aspects, the one or more inherited or acquired germ-line mutations comprise loss of XPD. In some aspects, the one or more inherited or acquired germ-line mutations comprise loss of XPE. In some aspects, the one or more inherited or acquired germ-line mutations comprise loss of XPF. In some aspects, the one or more inherited or acquired germ-line mutations comprise loss of XPG. In some aspects, the one or more inherited or acquired germ-line mutations comprise loss of MSH2. In some aspects, the one or more inherited or acquired germ-line mutations comprise loss of MLH1. In some aspects, the one or more inherited or acquired germ-line mutations comprise loss of MSH6. In some aspects, the one or more inherited or acquired germ-line mutations comprise loss of PMS2. In some aspects, the one or more inherited or acquired germ-line mutations comprise loss of WRN. In some aspects, the one or more inherited or acquired germ-line mutations comprise loss of BLM. In some aspects, the one or more inherited or acquired germ-line mutations comprise loss of FANCA. In some aspects, the one or more inherited or acquired germ-line mutations comprise loss of FANCB. In some aspects, the one or more inherited or acquired germ-line mutations comprise loss of FANCC. In some aspects, the one or more inherited or acquired germ-line mutations comprise loss of FANCD2. In some aspects, the one or more inherited or acquired germ-line mutations comprise loss of FANCE. In some aspects, the one or more inherited or acquired germ-line mutations comprise loss of FANCF. In some aspects, the one or more inherited or acquired germ-line mutations comprise loss of FANCG. In some aspects, the one or more inherited or acquired germ-line mutations comprise loss of FANCI. In some aspects, the one or more inherited or acquired germ-line mutations comprise loss of FANCJ. In some aspects, the one or more inherited or acquired germ-line mutations comprise loss of FANCL. In some aspects, the one or more inherited or acquired germ-line mutations comprise loss of FANCM. In some aspects, the one or more inherited or acquired germ-line mutations comprise loss of FANCN. In some aspects, the one or more inherited or acquired germ-line mutations comprise loss of FANCO. In some aspects, the one or more inherited or acquired germ-line mutations comprise loss of FANCP. In some aspects, the one or more inherited or acquired germ-line mutations comprise loss of FANCQ. In some aspects, the one or more inherited or acquired germ-line mutations comprise loss of FANCT. In some aspects, the one or more inherited or acquired germ-line mutations comprise loss of FANCU. In some aspects, the one or more inherited or acquired germ-line mutations comprise loss of FANCV. In some aspects, the one or more inherited or acquired germ-line mutations comprise loss of FANCW. In some aspects, the one or more inherited or acquired germ-line mutations comprise loss of NBS. In some aspects, the one or more inherited or acquired germ-line mutations comprise loss of Chek2. In some aspects, the one or more inherited or acquired germ-line mutations comprise loss of RecqL4. In some aspects, the one or more inherited or acquired germ-line mutations comprise loss of MYH.

In some aspects, the one or more inherited or acquired germ-line mutations comprise loss of PALB2. In some aspects, the one or more inherited or acquired germ-line mutations comprise loss of BACH1. In some aspects, the one or more inherited or acquired germ-line mutations comprise loss of RAC51C.

In another aspect, a method is provided for treating an infection resulting from a papillomavirus in a subject in need thereof comprising administering a therapeutically effective amount of a compound described herein, or a pharmaceutically acceptable salt thereof, to the subject.

In some aspects, the papillomavirus is human papillomavirus (HPV). In some aspects, the HPV is an HPV strain selected from a strain including, but not limited to, HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, HPV82, or any other HPV strain which is known to result in an infection associated with a medical disorder.

In another aspect, a method is provided for treating a medical disorder associated with infection with human papillomavirus comprising administering to a subject in need thereof a therapeutically effective amount of a compound described herein, or a pharmaceutically acceptable salt thereof. In some aspects, the medical disorder associated infection with human papillomavirus is cancer. Representative examples of medical disorders resulting from infection with HPV include, but are not limited to, common warts (associated with HPV2, HPV7, and HPV22, for example), plantar warts (associated with HPV1, HPV2, HPV4, and HPV63, for example), flat warts (associated with HPV3, HPV10, and HPV28, for example), anogenital warts (associated with HPV6, HPV11, HPV42, and HPV42, for example), genital cancers (associated with HPV16, HPV18, HPV26 HPV31, HPV33, HPV35, HPV39, HPV45, HPV51, HPV52, HPV53, HPV56, HPV58, HPV59, HPV66, HPV72, and HPV82, for example), epidermodysplasia verruciformis, focal epithelial hyperplasia (associated with HPV13 and HPV32, for example), mouth papillomas (associated with HPV6, HPV7, HPV11, HPV16, and HPV32, for example), oropharyngeal cancer (associated with HPV16, for example), verrucous cyst (associated with HPV60, for example), and laryngeal papillomatosis (associated with HPV6 and HPV11, for example).

In some aspects, the one or more additional therapeutic agents may comprise a Chk1 inhibitor, an ATR inhibitor, a Cdc7 inhibitor, or a Parp inhibitor.

In another aspect, a method for treating cancer in a subject in need thereof is provided, the method comprising:
(a) administering a therapeutically effective amount of a compound of Formula I, or a pharmaceutically acceptable salt thereof; and
(b) administering one or more additional therapeutic agents selected from a Chk1 inhibitor, an ATR inhibitor, a Cdc7 inhibitor, and a Parp inhibitor.

Representative Chk1 inhibitors which may be used in the above methods include, but are not limited to, AZD7762, Rabusertib (LY2603618), MK-8776 (SCH 900776), CHIR-124, PF-477736, prexasertib (LY2606368), GDC-0575, SAR-020106, CCT245737, and PD166285.

Representative ATR inhibitors which may be used in the above methods include, but are not limited to, VE-821, Berzosertib (VE-822), elimusertib (BAY-1895344), ETP-46464, CGK 733, AZ20, AZ31, ceralasertib (AZD6738), and VX-803 (M4344).

Representative examples of Cdc7 inhibitors which may be used in the above methods include, but are not limited to, XL-413, PHA-767491 (CAY10572), and LY3143921.

Representative examples of Parp inhibitors which may be used in the above methods include, but are not limited to, Olaparib, rucaparib, niraparib, talazoparib, veliparib, pamiparib (BGB-290), CEP 9722, E7016, 3-aminobenzamide, fluzoparib, AG-14361, A-966492, PJ34, UPF 1069, AZD2461, ME0328, BYK204165, BGP-15, RBN-2397, NU1025, E7449, 4-hydroxyquinazoline, NMS-P118, RBN012759, and picolinamide.

In view of the described compounds, compositions, and methods, hereinbelow are described certain more particular aspects of the disclosure. These particularly recited aspects should not, however, be interpreted to have any limiting effect on any different claims containing different or more general teachings described herein, or that the “particular” aspects are somehow limited in some way other than the inherent meanings of the language and formulae literally used therein.

Aspect 1. A compound of Formula I

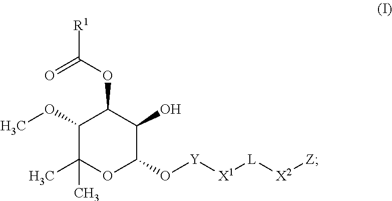

or a pharmaceutically acceptable salt thereof;
wherein:
R¹ is selected from monocyclic or bicyclic heteroaryl and monocyclic or bicyclic heterocycloalkyl, wherein each R¹ optionally includes at least one ring nitrogen atom substituted with R⁷ as allowed by valency, and wherein R¹ is optionally substituted with 1, 2, 3, or 4 groups selected from R⁸ as allowed by valency;
Y is selected from a bond, bicyclic aryl, or bicyclic heteroaryl, wherein Y is optionally substituted with 1, 2, 3, or 4 groups independently selected from R² as allowed by valency;
X¹ is selected from a bond or —NR^{a}—;
L is selected from a bond or -L¹-L²-L³-L⁴-L⁵-L⁶-L⁷-;
L¹, L², L³, L⁴, L⁵, L⁶, and L⁷ are independently selected from:
a) a bond;
b) —C(═O)—;
c) —C≡C—;
d) —NR^{a}—;
e) —O—;
d) C₁-C₁₀ alkyl;
e) cycloalkyl optionally substituted with 1, 2, 3, or 4 substituents independently selected from R³;
f) heterocycloalkyl optionally substituted with 1, 2, 3, or 4 substituents independently selected from R⁴;
g) aryl optionally substituted with 1, 2, 3, or 4 groups independently selected from R⁵;
h) heteroaryl optionally substituted with 1, 2, 3, or 4 groups independently selected from R⁶;
i) —NR^{a}(C═O)—;
j) —C(═O)NR^{a}—;
k) —C(═O)(C₁-C₆ alkyl)-;
l) —(C₁-C₆ alkyl)C(═O)—;
m) —(C₁-C₆ alkyl)NR^{a}—;
n) —NR^{a}(C₁-C₆ alkyl)-;
o) —(C₁-C₆ alkyl)O—; and
p) —O(C₁-C₆ alkyl)-;
X² is selected from a bond, —NR^{a}—, —O—, —C≡C—, —C(═O)—, —S(═O)₂—, —C(═O)NR^{a}—, —NR^{a}C(═O)—, —S(═O)₂NR^{a}—, —NR^{a}S(═O)₂—, and —NR^{a}(C═O)NR^{b}—;
Z is an E3 ubiquitin ligase binding moiety;
R² is selected at each occurrence from halogen, hydroxy, nitro, —NHR^{a}, C₁-C₆ alkyl, C₁-C₆ haloalkyl, aryl, and oxo;
R³ and R⁴ are independently selected at each occurrence from halogen, hydroxy, —NHR^{a}, C₁-C₆ alkyl, C₁-C₆ haloalkyl and oxo;
R⁵ and R⁶ are independently selected at each occurrence from halogen, hydroxy, nitro, —NHR^{a}, C₁-C₆ alkyl, C₁-C₆ haloalkyl, and aryl;
R⁷ is selected from: hydrogen; C₁-C₄ alkyl; C₂-C₆ alkenyl; C₂-C₆ alkynyl; C₃-C₆ cycloalkyl; C₁-C₆ alkoxy; and (C₀-C₆ alkyl)(aryl) which is optionally substituted with halogen, C₁-C₆ alkyl, or C₁-C₆ alkoxy;
R⁸ is selected from: hydrogen; halogen; hydroxy; —NR^{a}R^{b}; nitro; C₁-C₆ alkyl; C₁-C₆ haloalkyl; C₃-C₆ cycloalkyl; C₃-C₆ heterocycloalkyl; C₂-C₆ alkenyl; C₂-C₆ alkynyl; C₁-C₆ alkoxy; (C₀-C₆ alkyl)(aryl) which is optionally substituted with halogen, C₁-C₆ alkyl, or C₁-C₆ alkoxy; and (C₀-C₆ alkyl)(heteroaryl) which is optionally substituted with halogen, C₁-C₆ alkyl, or C₁-C₆ alkoxy; and
R^{a} and R^{b} are independently selected at each occurrence from hydrogen and C₁-C₆ alkyl.

Aspect 2. The compound of aspect 1, wherein Z is selected from:

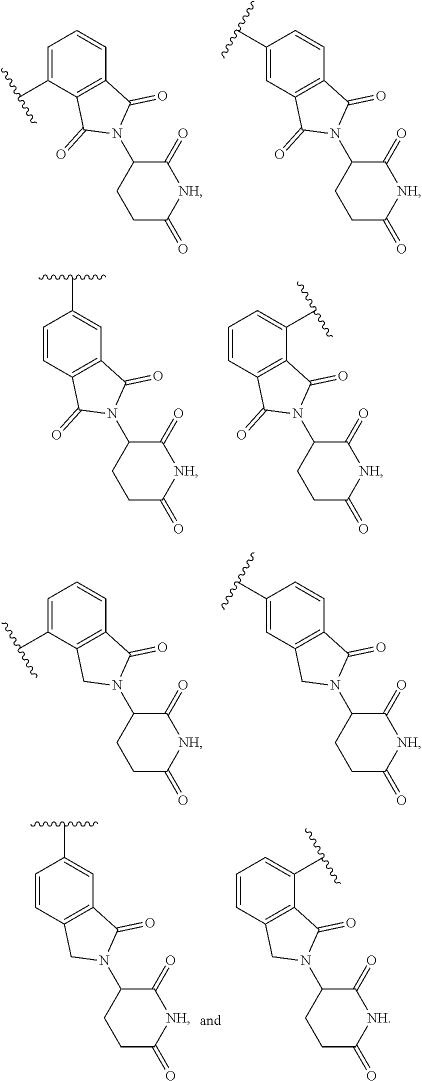

Aspect 3. The compound of aspect 1, wherein Z is

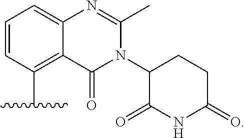 [SMILES: *c1cccc2nc(C)n(C3CCC(=O)NC3=O)c(=O)c12]

Aspect 4. The compound of aspect 1, wherein Z is selected from:

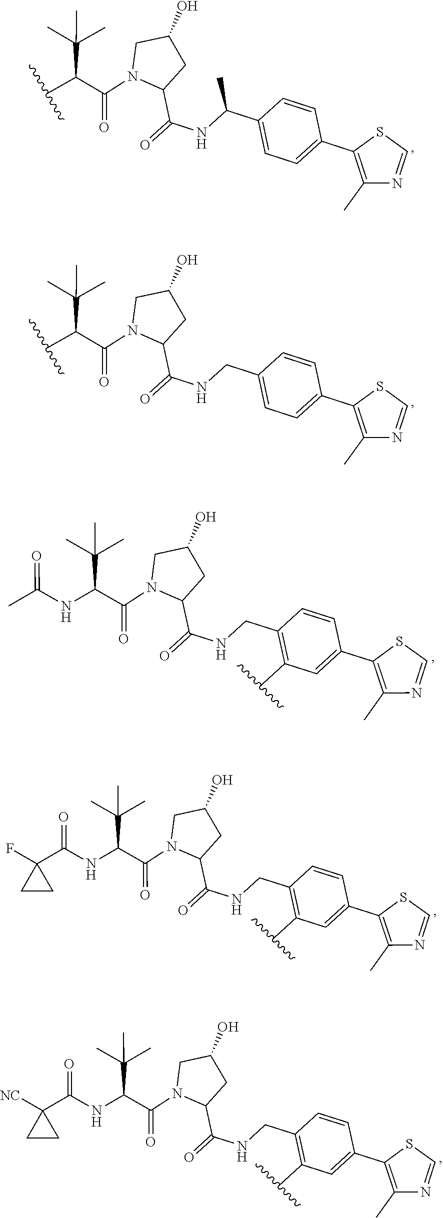

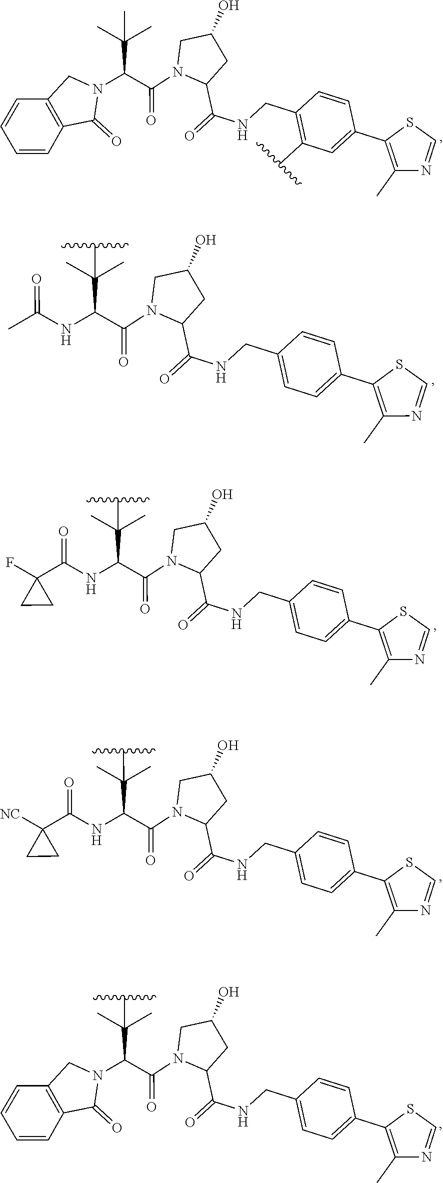

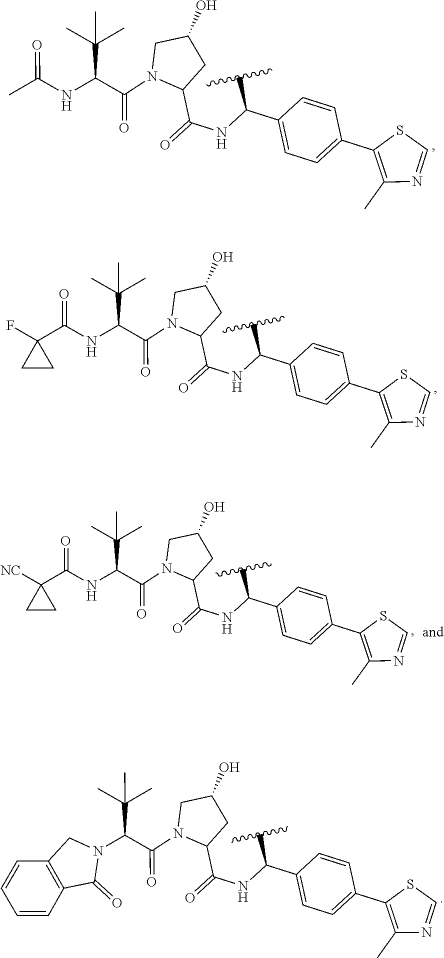

Aspect 5. The compound of aspect 1, wherein Z is

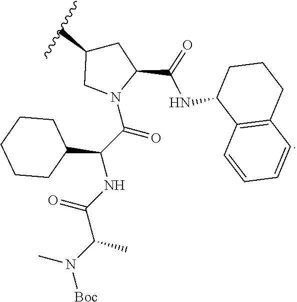 [SMILES: *[C@H]1C[C@@H](C(=O)N[C@@H]2CCCc3ccccc32)N(C(=O)[C@@H](NC(=O)[C@H](C)N(C)C(=O)OC(C)(C)C)C2CCCCC2)C1]

Aspect 6. The compound of any one of aspects 1-5, wherein R¹ is

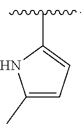 [SMILES: *c1ccc(C)[nH]1]

Aspect 7. The compound of any one of aspects 1-5, wherein R¹ is selected from:

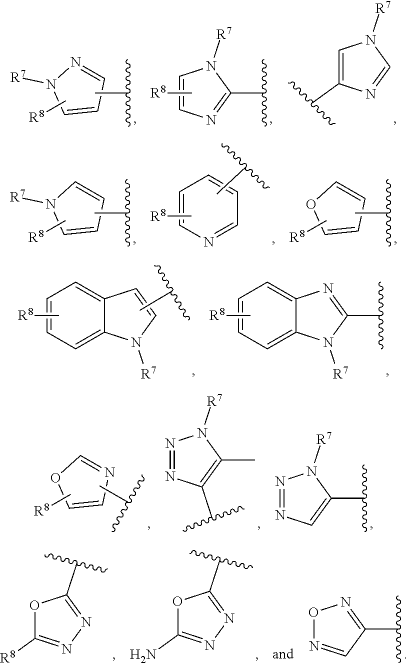

Aspect 8. The compound of any one of aspects 1-5, wherein R¹ is selected from:

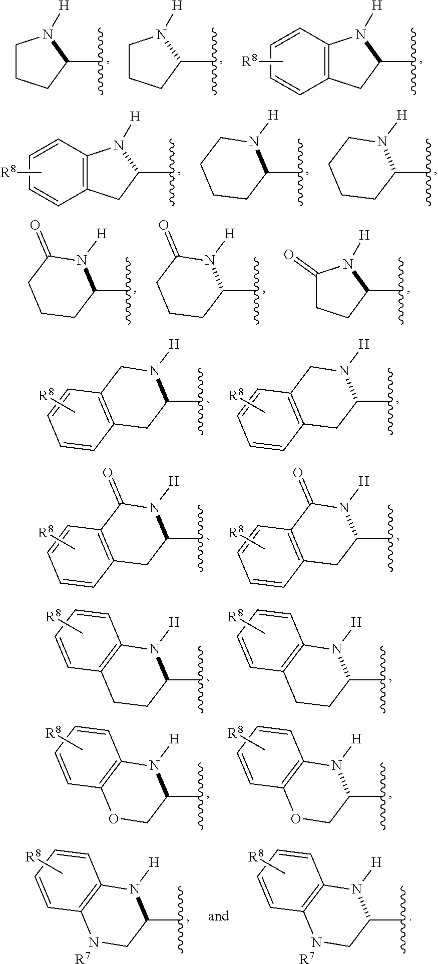

Aspect 9. The compound of any one of aspects 1-8, wherein Y is a bond.

Aspect 10. The compound of any one of aspects 1-8, wherein Y is

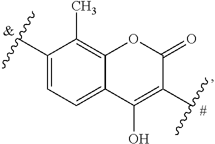 [SMILES: *c1ccc2c(O)c(*)c(=O)oc2c1C]

wherein & denotes the point of attachment to the neighboring oxygen atom and # denotes the point of attachment to X¹ and/or X².

Aspect 11. The compound of any one of aspects 1-8, wherein Y is selected from:

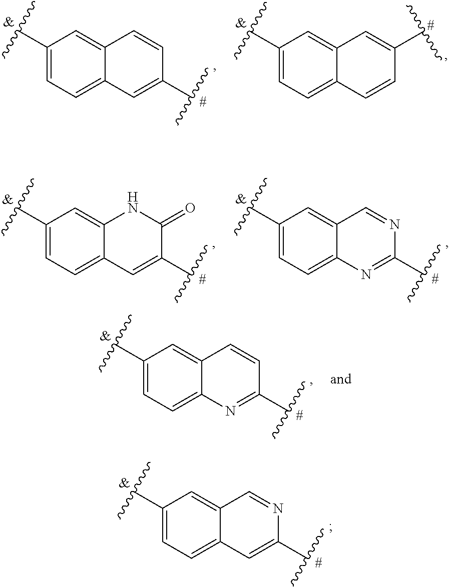 [SMILES: *c1ccc2cc(*)ccc2c1 ; *c1ccc2ccc(*)cc2c1 ; *c1ccc2cc(*)c(=O)[nH]c2c1 ; *c1ccc2nc(*)ncc2c1 ; *c1ccc2nc(*)ccc2c1 ; *c1ccc2cc(*)ncc2c1]

wherein & denotes the point of attachment to the neighboring oxygen atom and # denotes the point of attachment to X¹ and/or X².

Aspect 12. The compound of any one of aspects 1-11, wherein X¹ is —NH—.

Aspect 13. The compound of any one of aspects 1-12, wherein L¹, L², L³, L⁴, L⁵, L⁶, and L⁷ are each independently selected from: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—;

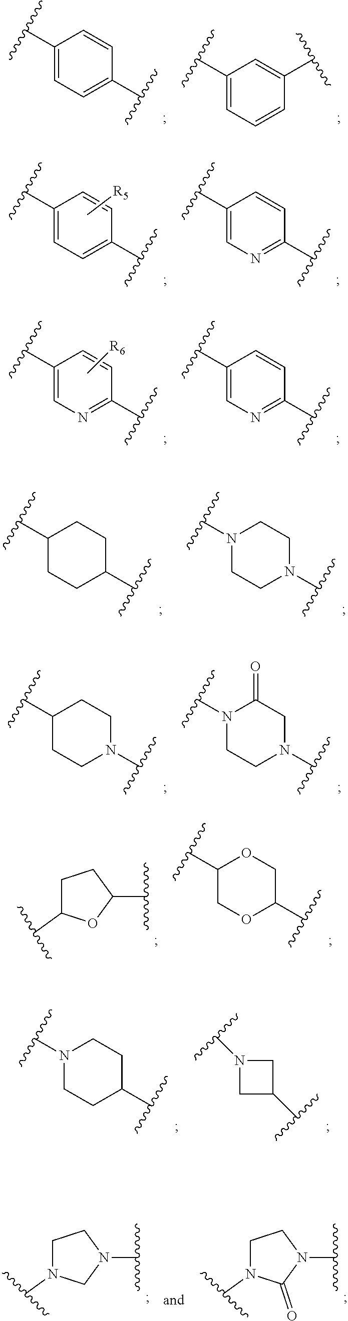

wherein L¹, L², L³, L⁴, L⁵, L⁶, and L⁷ are selected in such a way that: no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of L¹, L², L³, L⁴, L⁵, L⁶, and L⁷.

Aspect 14. The compound of any one of aspects 1-12, wherein L is selected from:

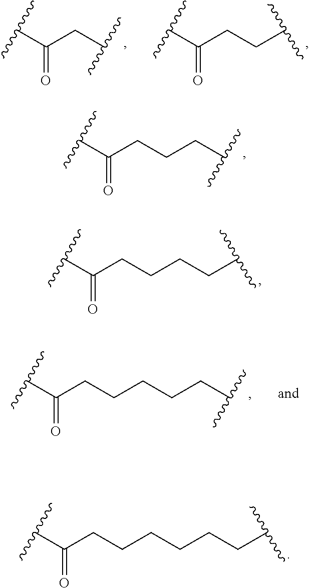 [SMILES: *CC(*)=O ; *CCC(*)=O ; *CCCC(*)=O ; *CCCCC(*)=O ; *CCCCCC(*)=O ; *CCCCCCC(*)=O]

Aspect 15. The compound of any one of aspects 1-12, wherein L is selected from:

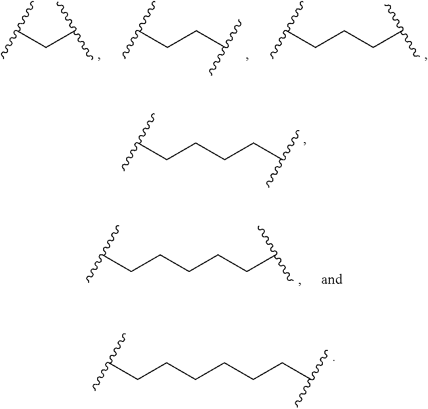

Aspect 16. The compound of any one of aspects 1-12, wherein L is selected from:

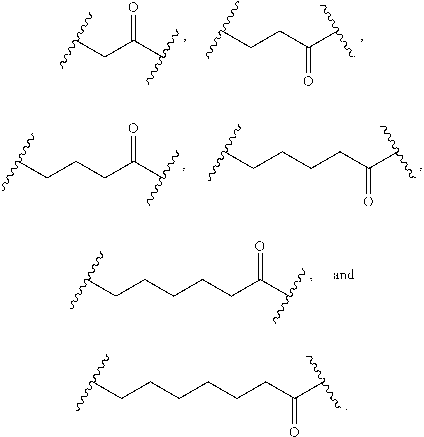 [SMILES: *CC(*)=O ; *CCC(*)=O ; *CCCC(*)=O ; *CCCCC(*)=O ; *CCCCCC(*)=O ; *CCCCCCC(*)=O]

Aspect 17. The compound of any one of aspects 1-12, wherein L is

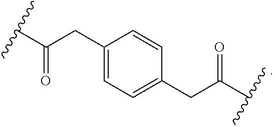 [SMILES: *C(=O)Cc1ccc(CC(*)=O)cc1]

Aspect 18. The compound of any one of aspects 1-12, wherein L is

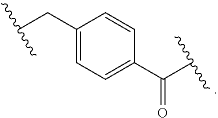 [SMILES: *Cc1ccc(C(*)=O)cc1]

Aspect 19. The compound of any one of aspects 1-12, wherein L is selected from:

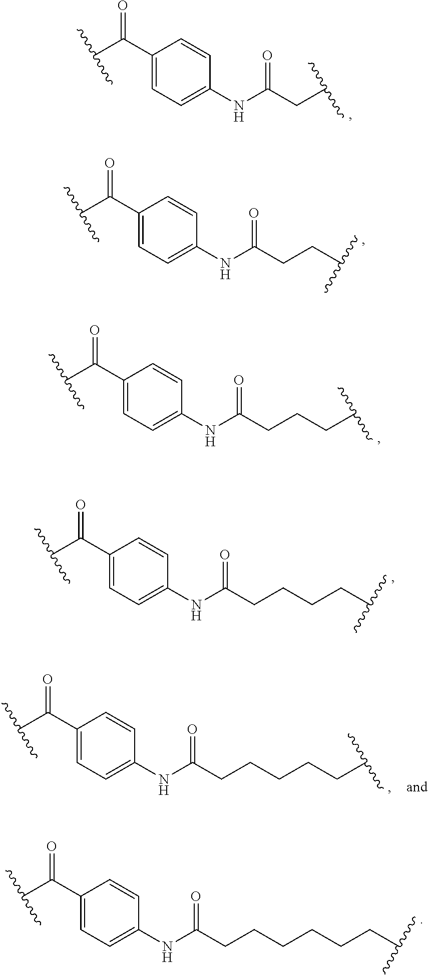 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

Aspect 20. The compound of any one of aspects 1-12, wherein L is selected from:

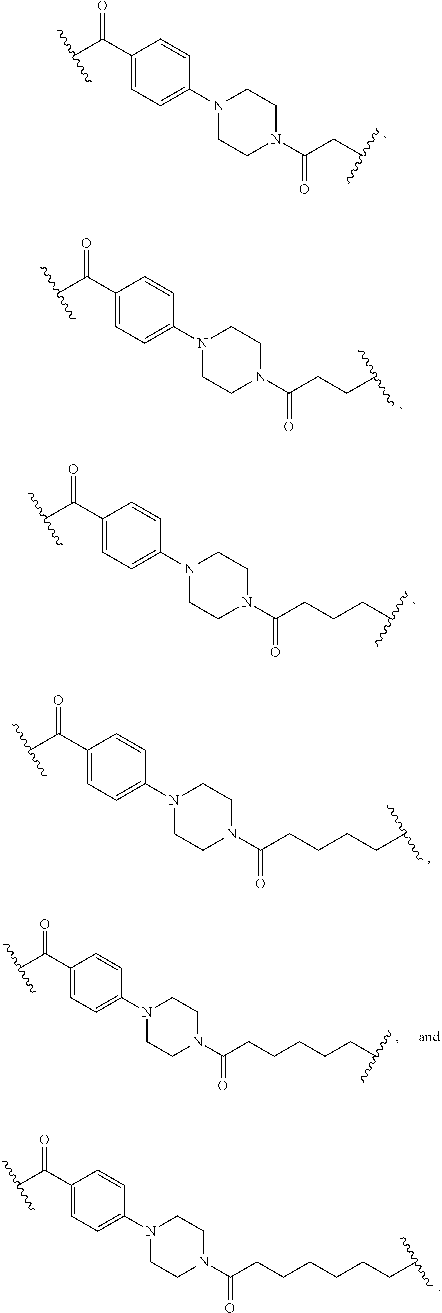 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

Aspect 21. The compound of any one of aspects 1-12, wherein L is selected from:

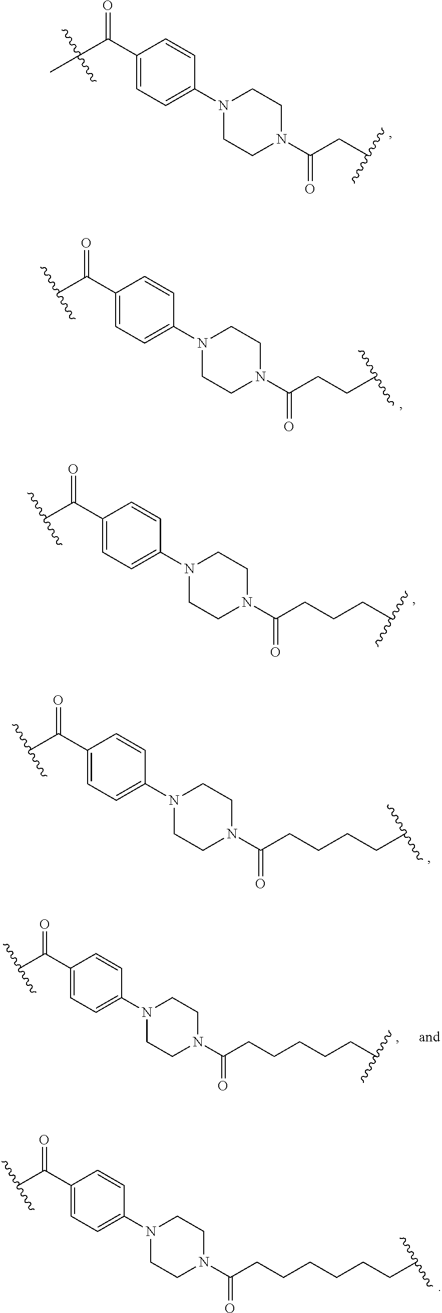 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

Aspect 22. The compound of any one of aspects 1-12, wherein L is selected from:

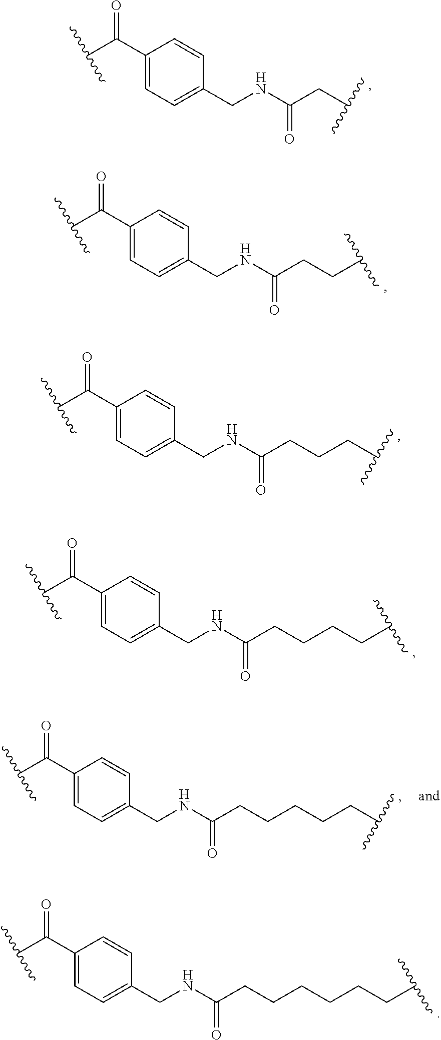 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

Aspect 23. The compound of any one of aspects 1-12, wherein L is selected from:

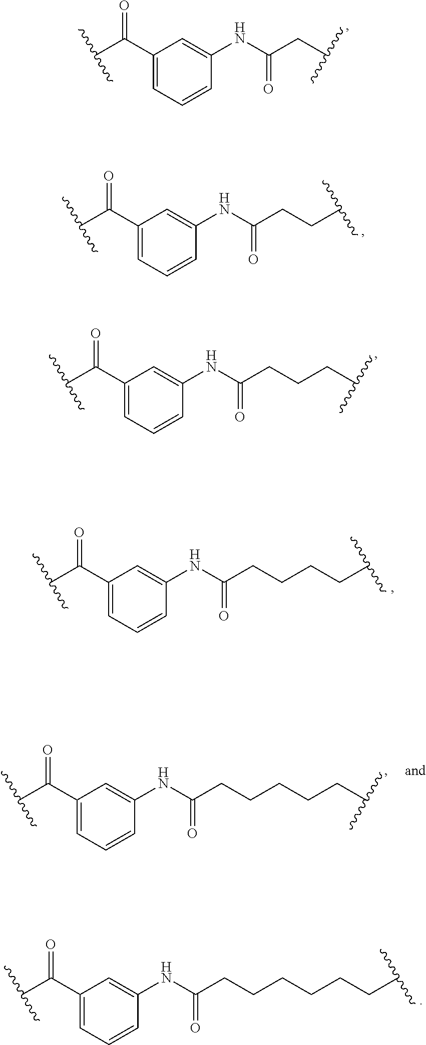 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

Aspect 24. The compound of any one of aspects 1-12, wherein L is selected from:

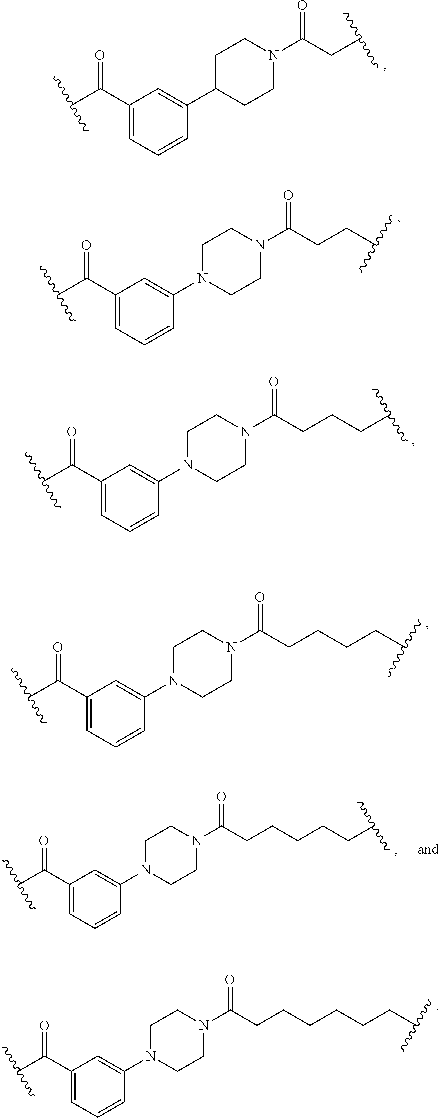 [SMILES: *CC(=O)N1CCC(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

Aspect 25. The compound of any one of aspects 1-12, wherein L is selected from:

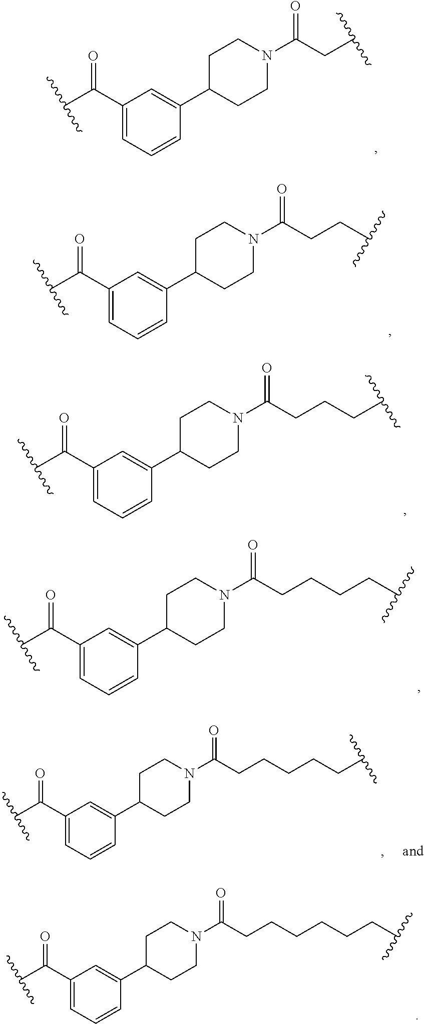 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

Aspect 26. The compound of any one of aspects 1-12, wherein L is selected from:

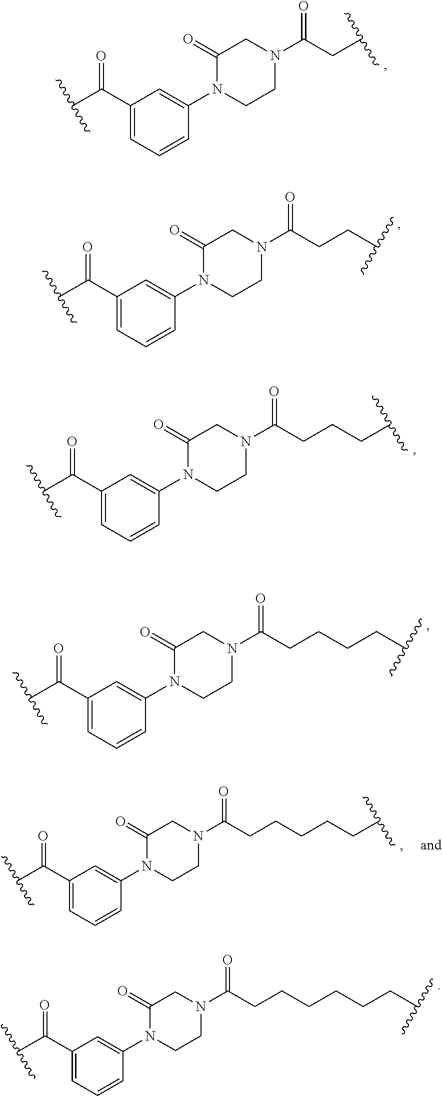 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

Aspect 27. The compound of any one of aspects 1-12, wherein L is selected from:

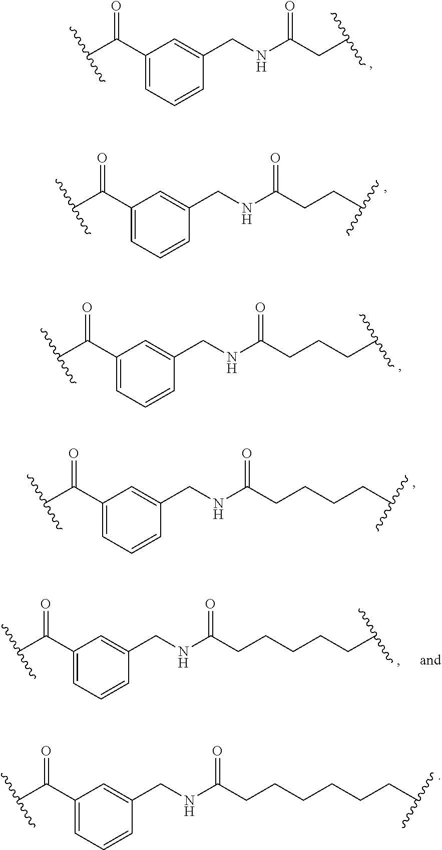 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCCC(=O)NCc1cccc(C(*)=O)c1 ; *CCCCCCC(=O)NCc1cccc(C(*)=O)c1]

Aspect 28. The compound of any one of aspects 1-12, wherein L is selected from:

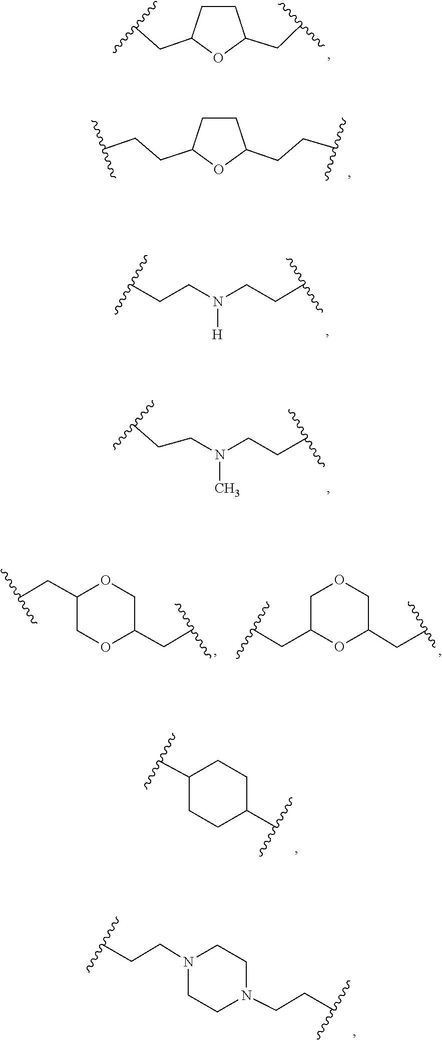

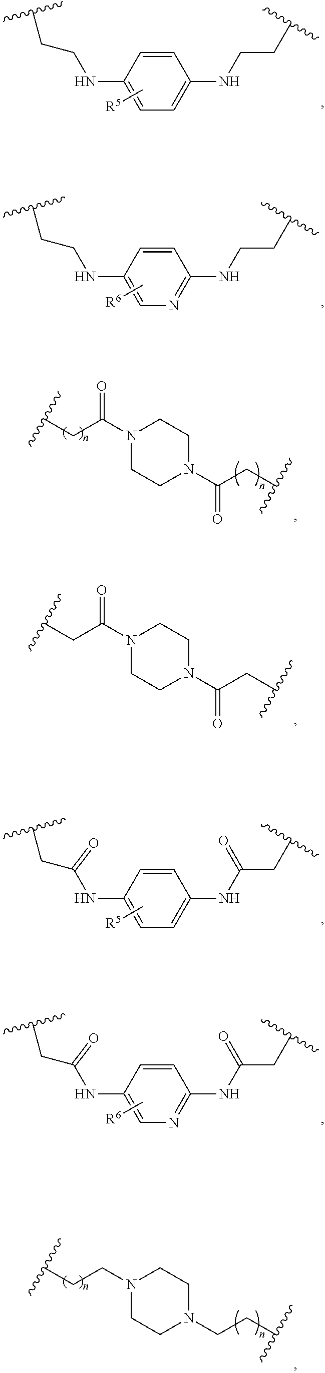

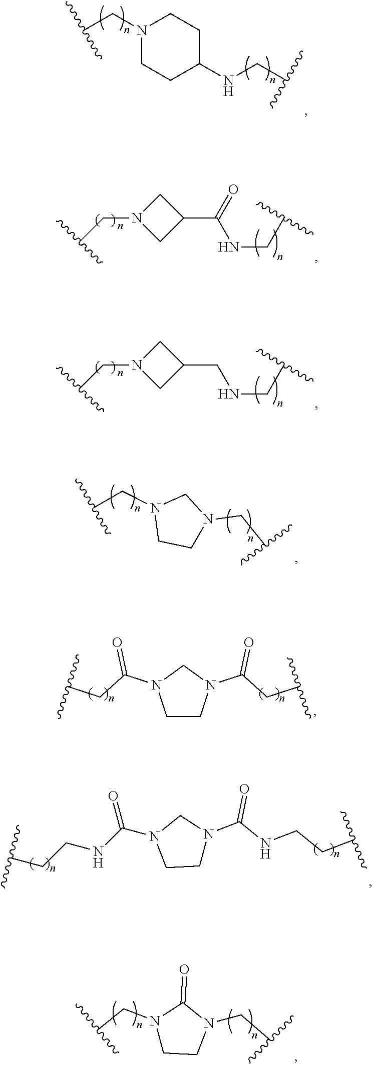

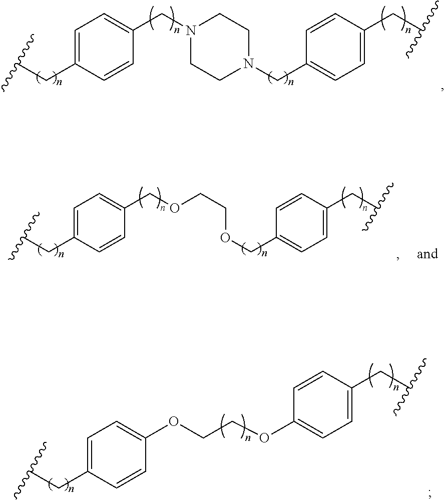

wherein n is independently selected at each occurrence from 1, 2, 3, 4, 5, and 6.

Aspect 29. The compound of any one of aspects 1-28, wherein X² is a bond.

Aspect 30. The compound of any one of aspects 1-28, wherein X² is —NH—.

Aspect 31. The compound of any one of aspects 1-28, wherein X² is —O—.

Aspect 32. The compound of any one of aspects 1-28, wherein X² is —C≡C—.

Aspect 33. The compound of any one of aspects 1-28, wherein X² is —C(═O)—.

Aspect 34. The compound of any one of aspects 1-28, wherein X² is —S(═O)₂—.

Aspect 35. The compound of any one of aspects 1-28, wherein X² is —C(═O)NH—.

Aspect 36. The compound of any one of aspects 1-28, wherein X² is —NHC(═O)—.

Aspect 37. The compound of any one of aspects 1-28, wherein X² is —S(═O)₂NH—.

Aspect 38. The compound of any one of aspects 1-28, wherein X² is —NHS(═O)₂—.

Aspect 39. The compound of any one of aspects 1-28, wherein X² is —NH(C═O)NH—.

Aspect 40. A pharmaceutical composition comprising a compound of any one of aspects 1-39, or a pharmaceutically acceptable salt thereof, and a pharmaceutically acceptable carrier or excipient.

Aspect 41. A method for treating cancer in a subject in need thereof comprising administering to the subject a therapeutically effective amount of a compound of any one of aspects 1-39, or a pharmaceutically acceptable salt thereof, or a pharmaceutical composition of aspect 40.

Aspect 42. The method of aspect 41, wherein the cancer is associated with or mediated by a helicase.

Aspect 43. The method of aspect 42, wherein the helicase is an SF3 helicase.

Aspect 44. The method of aspect 43, wherein the helicase is HPV E1 helicase.

Aspect 45. The method of aspect 42, wherein the helicase is an SF6 helicase.

Aspect 46. The method of aspect 45, wherein the helicase is CMG helicase.

Aspect 47. The method of any one of aspects 41 or 42, wherein the cancer is associated with overactivation of CMG helicase.

Aspect 48. The method of any one of aspects 41 or 42, wherein the cancer is associated with an infection by a papillomavirus.

Aspect 49. The method of aspect 48, wherein the papillomavirus is human papillomavirus (HPV).

Aspect 50. A method for treating cancer in a subject in need thereof comprising:
(a) determining whether the cancer is associated with elevated expression of Myc and/or elevated expression of Cyclin E; and
(b) if the cancer is determined to be associated with elevated expression of Myc and/or elevated expression of Cyclin E in (a), administering a therapeutically effective amount of a compound of any one of aspects 1-39, or a pharmaceutically acceptable salt thereof, or a pharmaceutical composition of aspect 40.

Aspect 51. A method for treating cancer in a subject in need thereof, wherein the cancer has been previously determined to be associated with elevated expression of Myc and/or elevated expression of Cyclin E, the method comprising administering a therapeutically effective amount of a compound of any one of aspects 1-39, or a pharmaceutically acceptable salt thereof, or a pharmaceutical composition of aspect 40.

Aspect 52. The method of any one of aspects 50 or 51, wherein the cancer is associated with overactivation of CMG helicase.

Aspect 53. The method of any one of aspects 41-52, wherein the compound or pharmaceutical composition is administered in combination or alternation with one or more additional therapeutic agents.

Aspect 54. The method of aspect 53, wherein the one or more additional therapeutic agents are a chemotherapeutic or cytotoxic agent.

Aspect 55. A method of treating an infection with a papillomavirus in a subject in need thereof comprising administering a therapeutically effective amount of a compound of any one of aspects 1-39, or a pharmaceutically acceptable salt thereof, or a pharmaceutical composition of aspect 40.

Aspect 56. The method of aspect 55, wherein the papillomavirus comprises human papillomavirus.

Aspect 57. The method of aspect 56, wherein the human papillomavirus comprises a strain selected from HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, and HPV82.

Aspect 58. The method of aspect 56, wherein the human papillomavirus comprises a strain selected from HPV16, HPV18, HPV31, and HPV45.

Aspect 59. The method of any one of aspects 56-58, wherein the human papillomavirus is associated with a cancer.

Aspect 60. The method of aspect 59, wherein the cancer is selected from cervical cancer, vulvar cancer, vaginal cancer, penile cancer, anal cancer, rectal cancer, oropharyngeal cancer, and head and neck cancer.

Aspect 61. A method for degrading a helicase in a eukaryotic cell comprising contacting the cell with an effective amount of a compound of any one of aspects 1-39, or a pharmaceutically acceptable salt thereof.

Aspect 62. The method of aspect 61, wherein the helicase is an SF3 helicase.

Aspect 63. The method of any one of aspects 61 or 62, wherein the helicase is HPV E1 helicase.

Aspect 64. The method of aspect 61, wherein the helicase is an SF6 helicase.

Aspect 65. The method of any one of aspects 61 or 64, wherein the helicase is CMG helicase.

Aspect 66. A method for inhibiting replication of a papillomavirus in a eukaryotic cell comprising contacting the cell with an effective amount of a compound of any one of aspects 1-39, or a pharmaceutically acceptable salt thereof.

Aspect 67. The method of aspect 66, wherein the papillomavirus is human papillomavirus.

Aspect 68. The method of aspect 67, wherein the human papillomavirus comprises a strain selected from HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, and HPV82.

Aspect 69. The method of aspect 67, wherein the human papillomavirus comprises a strain selected from HPV16, HPV18, HPV31, and HPV45.

Aspect 70. The method of any one of aspects 61-69, wherein the eukaryotic cell is a human cell.

Aspect 71. a method for treating cancer in a subject in need thereof comprising:
(a) determining whether the cancer harbors one or more inherited or acquired germ-line mutations; and
(b) if the cancer is determined to harbor one or more inherited or acquired germ-line mutations in (a), administering to the subject a therapeutically effective amount of a compound of any one of claims 1-39, or a pharmaceutically acceptable salt thereof, or a pharmaceutical composition of claim 40.

Aspect 72. A method for treating cancer in a subject in need thereof, wherein the cancer has been previously determined to harbor one or more inherited or acquired germ-line mutations, the method comprising administering to the subject a therapeutically effective amount of a compound of any one of claims 1-39, or a pharmaceutically acceptable salt thereof, or a pharmaceutical composition of claim 40.

Aspect 73. The method of claim 71 or claim 72, wherein the one or more inherited or acquired germ-line mutations comprises loss of: p53, Rb, BRCA1, BRCA2, ATM, a xeroderma pigmentosum gene (such as XPA, XPB, XPC, XPD, XPE, XPF, or XPG), a mismatch repair gene (such as MSH2, MLH1, MSH6, PMS2), WRN, BLM, a Fanconi anemia gene (such as FANCA, FANCB, FANC, FANCD2, FANCE, FANCF, FANCG, FANCI, FANCJ, FANCL, FANCM, FANCN, FANCO, FANCP, FANCQ, FANCT, FANCU, FANCV, or FANCW), NBS, Chek2, RecqL4, MYH, PALB2, BACH1, RAC51C, or combinations thereof.

Aspect 74. The method of any one of claims 71-73, wherein the compound is administered in combination with an additional therapeutic agent.

Aspect 75. The method of claim 74, wherein the additional therapeutic agent is selected from a Chk1 inhibitor, an ATR inhibitor, a Cdc7 inhibitor, or a Parp inhibitor.

Aspect 76. A method for treating cancer in a subject in need thereof comprising:
(a) administering to the subject a therapeutically effective amount of a compound of any one of claims 1-39, or a pharmaceutically acceptable salt thereof, or a pharmaceutical composition of claim 40; and
(b) administering an additional therapeutic agent selected from a Chk1 inhibitor, an ATR inhibitor, a Cdc7 inhibitor, and a Parp inhibitor.

A number of aspects of the disclosure have been described. Nevertheless, it will be understood that various modifications may be made without departing from the spirit and scope of the disclosure. Accordingly, other aspects are within the scope of the following claims.

By way of non-limiting illustration, examples of certain aspects of the present disclosure are given below.

### EXAMPLES

The following examples are set forth below to illustrate the compounds, compositions, and methods claimed herein, along with associated methods and results according to the disclosed subject matter. These examples are not intended to be inclusive of all aspects of the subject matter disclosed herein, but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1. Degraders/PROTACs of CMG Helicase Using Coumermycin Analogs

The synthesis of coumermycin analogs 5 and 9 shown in Scheme 1 is carried out using known synthetic protocols from readily available Novobiocin (see Laurin P, Ferroud D, Klich M, Dupuis-Hamelin C, Mauvais P, Lassaigne P, Bonnefoy A, Musicki B. Synthesis and in vitro evaluation of novel highly potent coumarin inhibitors of gyrase B. Bioorganic & Medicinal Chemistry Letters. 1999; 9(14):2079-84; and WO01/87309). Under acidic reaction conditions in acetone, Novobiocin provides key intermediate 1. Hydrolysis of the carbamate group of 1 generates hydroxyacetonide intermediate 2. The pyrrole-anhydride building block 3 is synthesized from commercially available ethyl 5-methyl-1H-pyrrole-2-carboxylate via two steps; first hydrolysis of the ester to obtain the carboxylic acid and coupling the resulting acid via peptide coupling conditions (e.g. EDCI, DCM) to obtain the required anhydride 3 (see Olson S H, Slossberg L H. Synthesis of coumermycin A1. Tetrahedron Letters. 2003; 44(1):61-3). Acylation, catalyzed by tributylphosphine, of the hydroxyl group in the sugar fragment 2 with the pyrrole anhydride 3 provides pyrrole carbamate 4, which is subjected to acidic conditions to generate pyrrole-noviose 5. The coumermycin building block 6 shown in Scheme 2 is synthesized using known synthetic procedures (see Musicki B, Periers A-M, Laurin P, Ferroud D, Benedetti Y, Lachaud S, Chatreaux F, Haesslein J-L, Iltis A, Pierre C, Khider J, Tessot N, Airault M, Demassey J, Dupuis-Hamelin C, Lassaigne P, Bonnefoy A, Vicat P, Klich M. Improved antibacterial activities of coumarin antibiotics bearing 5′,5′-dialkylnoviose: biological activity of RU79115. Bioorganic & Medicinal Chemistry Letters. 2000; 10(15):1695-9) from readily available 2,4-dihydroxy-3-methylacetophenone 10, first by selectively protecting the 4-hydroxyl group with dihydropyran to generate the protected acetophenone 11 and further reaction with diethyl carbonate to provide the coumarin intermediate 12. The coumarin precursor 13 is generated from 12 by reacting with diphenylazodimethane and deprotection of the 4-hydroxyl group which generates required coumarin fragment 6. The coumarin intermediate 6 is used to generate compound 7 using Mitsunobu reaction conditions. The hydrogenation of compound 7 using Pd/C followed by diazotization of the resulting intermediate facilitates formation of key intermediate amino-coumarin 9 shown in Scheme 1.

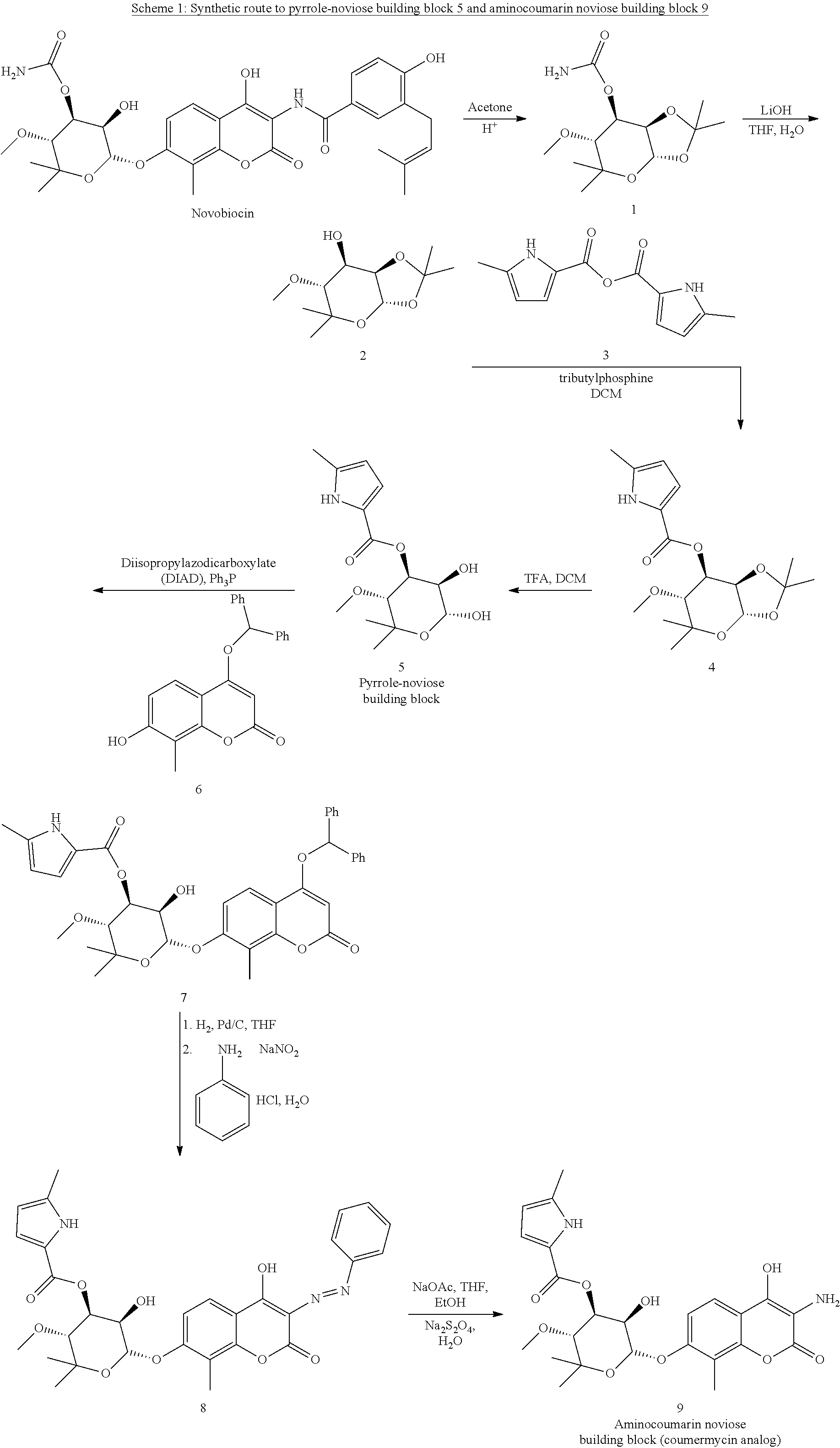

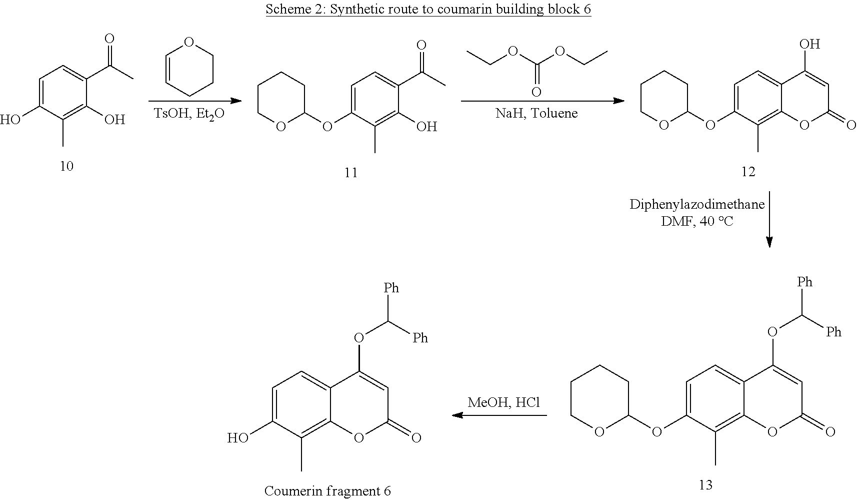

Synthesis of CMG helicase PROTACs

The lenalidomide (len) and pomalidomide (pom) E3-ligase warheads with carboxylic acid linker 15 and hydroxy linker 17 are synthesized as shown in Scheme 3, Panel A and B, respectively from readily available starting materials using known synthetic procedures (see WO2020014489). Commercially available len and pom are alkylated using bromo ester 14 and the resulting ester intermediates are hydrolyzed under basic conditions to obtain acid building blocks 15 (Panel A). The len and pom with hydroxyl linker 17 are synthesized via an alkylation reaction of len or pom with bromo-ether 16 and subsequent deprotection of the methoxy group with BBr₃ (Panel B) to obtain 17. The linker length of building blocks 15 and 17 are modified using appropriate bromo-carboxylic acid ester or bromo-ether building blocks shown in Panel A and Panel B respectively. The len and pom CRBN warheads are also easily replaced with thalidomide (thal) via modified reaction conditions using fluoro-thal with an alkylamine linker via nucleophilic replacement reaction (see Posternak G, Tang X, Maisonneuve P, Jin T, Lavoie H, Daou S, Orlicky S, Goullet de Rugy T, Caldwell L, Chan K, Aman A, Prakesch M, Poda G, Mader P, Wong C, Maier S, Kitaygorodsky J, Larsen B, Colwill K, Yin Z, Ceccarelli D F, Batey R A, Taipale M, Kurinov I, Uehling D, Wrana J, Durocher D, Gingras A C, Al-Awar R, Therrien M, Sicheri F. Functional characterization of a PROTAC directed against BRAF mutant V600E. Nature chemical biology. 2020; 16(11):1170-8) or bromo-thal and alkylpropargyl linker via Sonogoshira reaction conditions (Pd(PPh₃)₂Cl₂, CuI, Et₃N, DMF). The aminocoumarin 9 (Panel C) is coupled to carboxylic acid 15 using amide coupling conditions (see Carpino L A. 1-Hydroxy-7-azabenzotriazole. An Efficient Peptide Coupling Additive. Journal of the American Chemical Society. 1993; 115(14):4397-8) to obtain CMG helicase PROTAC 18. The pyrrole noviose 5 and hydroxyl intermediate 17 are alkylated under Mitsunobu conditions to obtain CMG helicase PROTAC 19 with CRBN warhead (Panel D). The CRBN warheads iberdomide [also known as CC220, (see Matyskiela M E, Zhang W, Man H W, Muller G, Khambatta G, Baculi F, Hickman M, LeBrun L, Pagarigan B, Carmel G, Lu C C, Lu G, Riley M, Satoh Y, Schafer P, Daniel T O, Carmichael J, Cathers B E, Chamberlain P P. A Cereblon Modulator (CC-220) with Improved Degradation of Ikaros and Aiolos. J Med Chem. 2018; 61(2):535-42; and Ye Y, Gaudy A, Schafer P, Thomas M, Weiss D, Chen N, Liu L, Xue Y, Carayannopoulos L, Palmisano M. First-in-Human, Single- and Multiple-Ascending-Dose Studies in Healthy Subjects to Assess Pharmacokinetics, Pharmacodynamics, and Safety/Tolerability of Iberdomide, a Novel Cereblon E3 Ligase Modulator. Clinical pharmacology in drug development. 2021; 10(5):471-85)] or avadomide [also known as CC122, (see Hagner P R, Man H W, Fontanillo C, Wang M, Couto S, Breider M, Bjorklund C, Havens C G, Lu G, Rychak E, Raymon H, Narla R K, Barnes L, Khambatta G, Chiu H, Kosek J, Kang J, Amantangelo M D, Waldman M, Lopez-Girona A, Cai T, Pourdehnad M, Trotter M, Daniel T O, Schafer P H, Klippel A, Thakurta A, Chopra R, Gandhi A K. CC-122, a pleiotropic pathway modifier, mimics an interferon response and has antitumor activity in DLBCL. Blood. 2015; 126(6):779-89; and Renneville A, Gasser J A, Grinshpun D E, Jean Beltran P M, Udeshi N D, Matyskiela M E, Clayton T, McConkey M, Viswanathan K, Tepper A, Guirguis A A, Sellar R S, Cotteret S, Marzac C, Saada V, De Botton S, Kiladjian J J, Cayuela J M, Rolfe M, Chamberlain P P, Carr S A, Ebert B L. Avadomide induces degradation of ZMYM2 fusion oncoproteins in hematologic malignancies. Blood cancer discovery. 2021; 2(3):250-65)] are occupied to replace the warheads in 18 and 19 (Scheme 3) to generate potent CMG helicase PROTACs The linker length of both compounds 18 and 19 (Scheme 3) is also modified to accommodate 4-15 atom linkers by using 4-15 atom linker building blocks (14 and 16). The in-vivo CMG helicase degradation potential of compounds 18 and 19 can then be optimized by modifying the linker length and CRBN ligands (Thal, CC220, CC122) mentioned above. Alternative linkers can also provide novel drug-like CMG-helicase PROTACS.

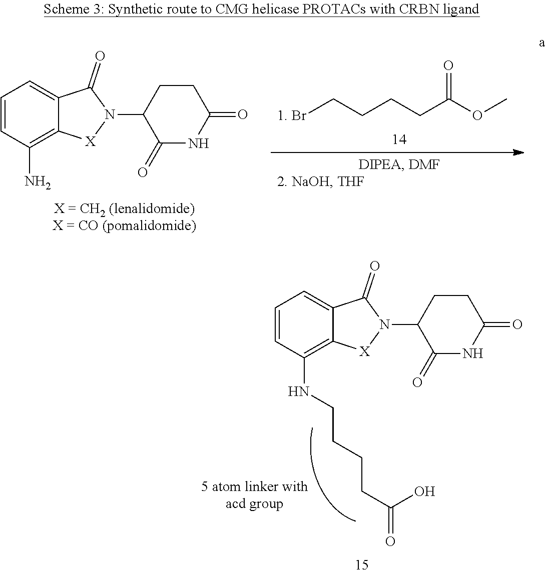

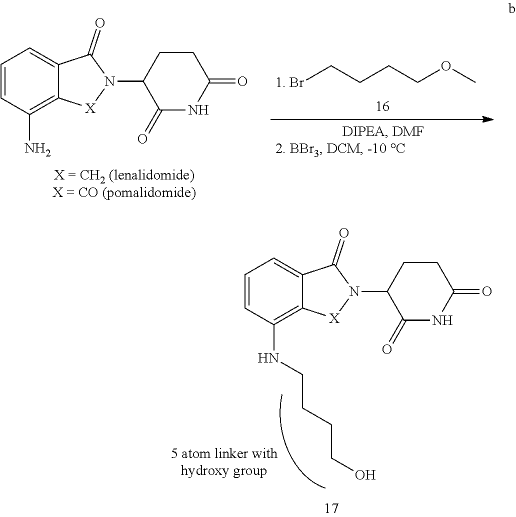

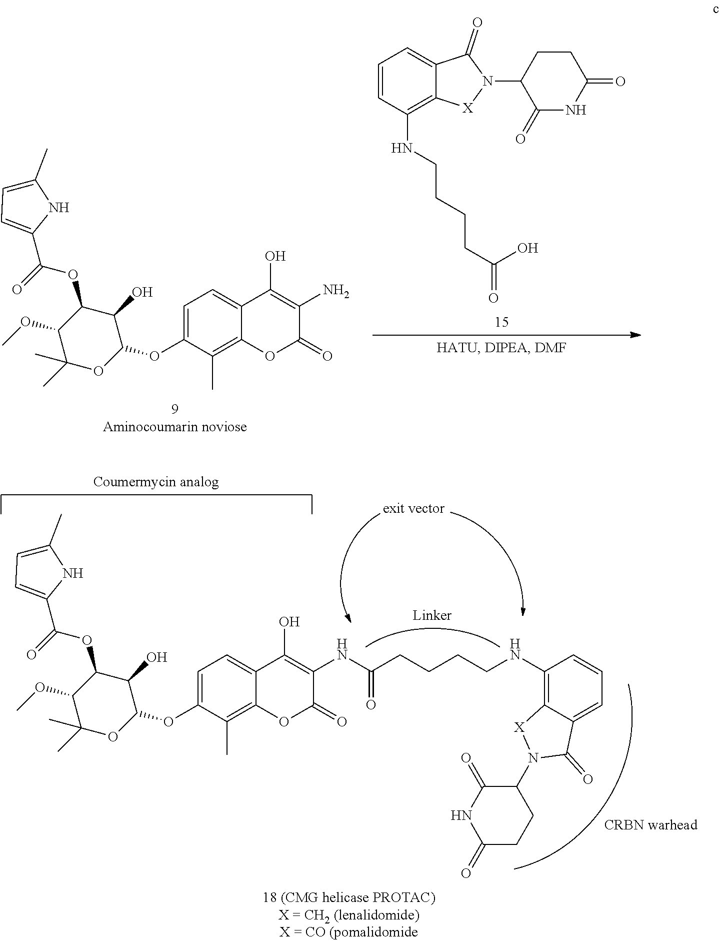

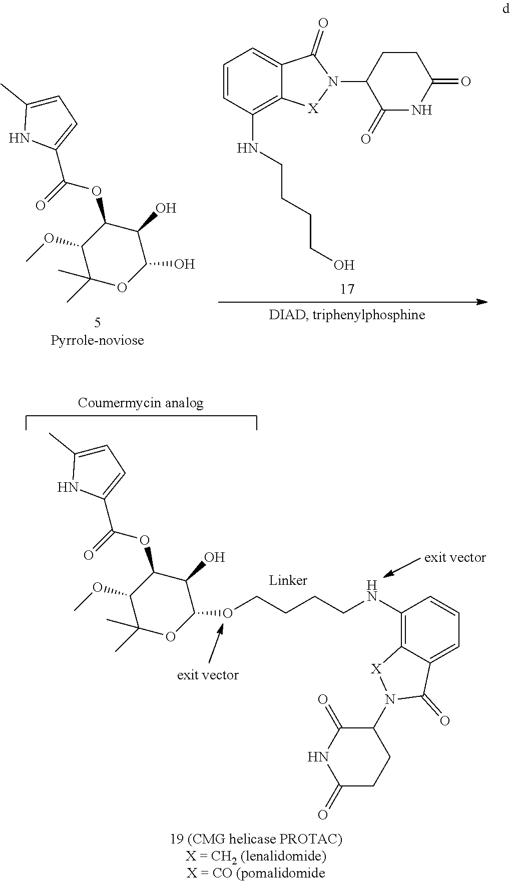

The synthetic route for CMG helicase PROTACs with IAP or VHL ligands is shown in Scheme 4 and begins with alkylation of coumarin building block 9 with bromo-ester 14 and hydrolysis of the subsequent product to form carboxylic acid intermediate 20. The IAP ligand 21a (see Ma Z, Ji Y, Yu Y, Liang D. Specific non-genetic IAP-based protein erasers (SNIPERs) as a potential therapeutic strategy. European journal of medicinal chemistry. 2021; 216:113247; and Tsukumo Y, Tsuji G, Yokoo H, Shibata N, Ohoka N, Demizu Y, Naito M. Protocols for Synthesis of SNIPERs and the Methods to Evaluate the Anticancer Effects. Methods in molecular biology (Clifton, NJ). 2021; 2365:331-47) is coupled with acid intermediate 20 using peptide coupling conditions followed by deprotection of the carbamate with TFA to obtain final CMG helicase PAOTAC 22a. The VHL ligand (see Girardini M, Maniaci C, Hughes S J, Testa A, Ciulli A. Cereblon versus VHL: Hijacking E3 ligases against each other using PROTACs. Bioorganic & medicinal chemistry. 2019; 27(12):2466-79) is coupled to acid intermediate 20 using peptide coupling conditions to obtain 22b. The CMG helicase PROTAC 24 with noviose-pyrrole is synthesized using Mitsunobu alkylation conditions to generate the carboxylic acid intermediate 23, which is then further coupled to IAP ligand or VHL ligand using peptide coupling conditions (HATU, DIPEA, DMF), and subsequent deprotection of the t-butoxycarbonyl group with TFA as shown in Scheme 4 to generate the final compounds.

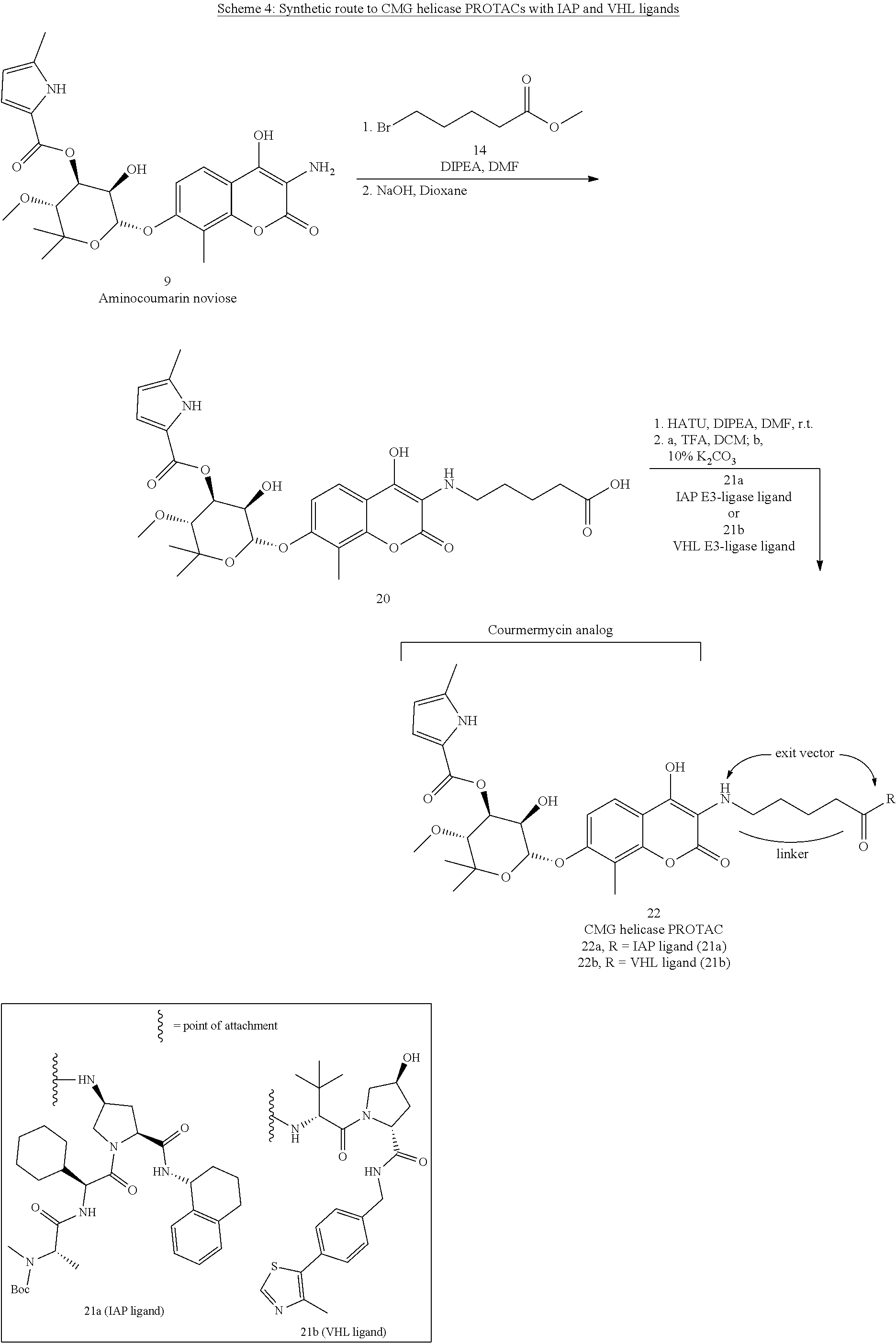

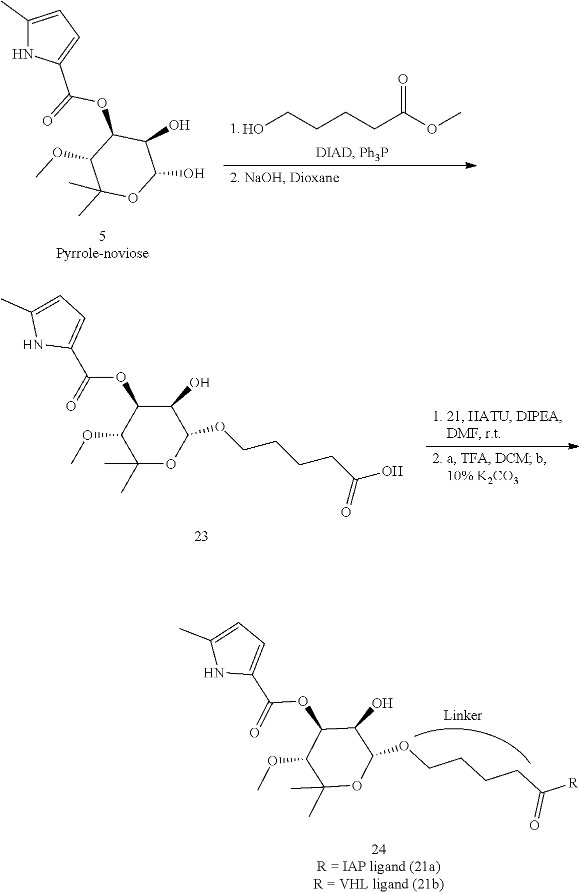

The synthetic routes to CMG helicase PROTACs with aromatic linkers are shown in Scheme 5. The coumarin 9 is coupled to commercially available benzene bis-carboxylic acid 25 using peptide coupling conditions to obtain intermediate 26 which is then coupled to VHL, IAP and CRBN warheads to obtain PROTAC compounds 27. The noviose PROTAC 30 is obtained by first reacting the pyrrole-noviose 5 and commercially available 4-hydroxymethylbenzene acetic acid 28 using Mitsunobu conditions to alkylate the sugar moiety followed by deprotection of the methyl ester to give intermediate 29. The acid 29 is then coupled to IAP, VHL, and CRBN warheads to generate PROTACs.

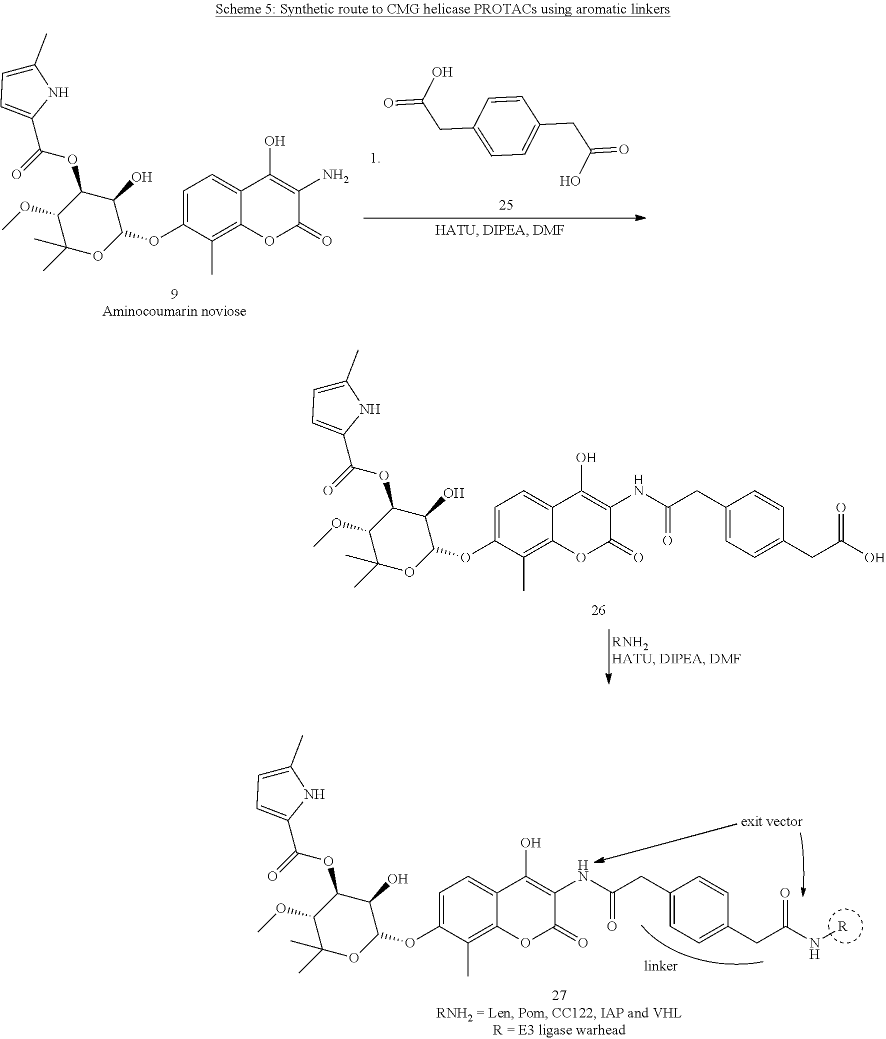

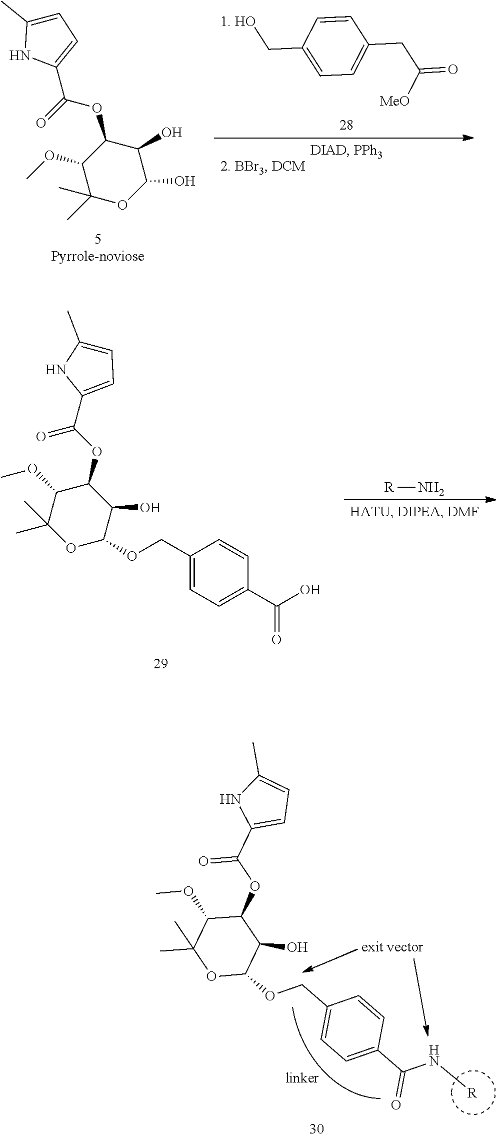

An alternative route to the key novenamine (9) shown in Scheme 1 utilizes the reported microbial-mediated benzamide hydrolysis of novobiocin (readily available as its sodium salt) (see Mandler M D, Baidin V, Lee J, Pahil K S, Owens T W, Kahne D. Novobiocin Enhances Polymyxin Activity by Stimulating Lipopolysaccharide Transport. Journal of the American Chemical Society. 2018; 140(22):6749-53). Alternatively, the benzamide group is cleaved by treatment with acetic anhydride and pyridine to provide the acetyloxazole 31 as reported (see Hinman J W, Caron E L, Hoeksema H. The Structure of Novobiocin. Journal of the American Chemical Society. 1957; 79(14):3789-800). Hydrolysis of the noviose acetyl group provides the oxazole 32. Treatment of the oxazole 32 with acetyl chloride in ethanol is reported to provide novenamine (9) in good yield (Ueda Y, Chuang J M, Crast L B, Partyka R A. A selective cleavage of the oxazole moiety in noviosylcoumarin antibiotics. A new process to key intermediates for coumermycin analog synthesis. The Journal of Organic Chemistry. 1988; 53(21):5107-13) (Scheme 6).

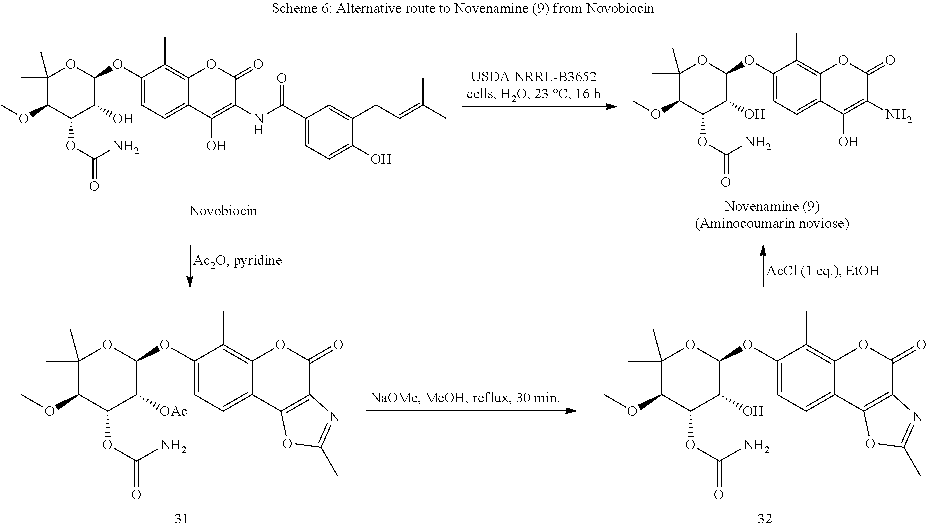 [SMILES: CO[C@@H]1[C@@H](OC(N)=O)[C@@H](O)[C@H](Oc2ccc3c(O)c(NC(=O)c4ccc(O)c(CC=C(C)C)c4)c(=O)oc3c2C)OC1(C)C ; CO[C@@H]1[C@@H](OC(N)=O)[C@@H](O)[C@H](Oc2ccc3c(O)c(N)c(=O)oc3c2C)OC1(C)C ; CO[C@@H]1[C@@H](OC(N)=O)[C@@H](OC(C)=O)[C@H](Oc2ccc3c(oc(=O)c4nc(C)oc43)c2C)OC1(C)C ; CO[C@@H]1[C@@H](OC(N)=O)[C@@H](O)[C@H](Oc2ccc3c(oc(=O)c4nc(C)oc43)c2C)OC1(C)C]

Novenamine (9) is known to be acylated via its coumarin amino group to provide amides (Scheme 7). Using HATU amide coupling conditions an N-protected benzoic acid of type 33 provides the benzamide 34. The C-3′ carbamate group of 34 is removed by treatment with sodium hydroxide to provide the free C-3′ alcohol 35 (Scheme 7) using conditions for similar noviose benzamides. Selective acylation of the C-3′ alcohol with 5-methyl-1H-pyrrole-2-carboxylic anhydride (3) (3) provides a key intermediate 36 possessing the pyrrole-noviose, coumarin and benzamide groups (present in coumermycin) (Scheme 7). Unmasking of the protected amine group of 36 provides an aniline of type 37. This aniline is coupled to an E3-ligase ligand conjugated to a linker with a terminal carboxylic acid, as represented by an acid 38 (see WO2020200291) which incorporates a thalidomide-like cereblon ligand to provide the helicase degrader amide of type 39.

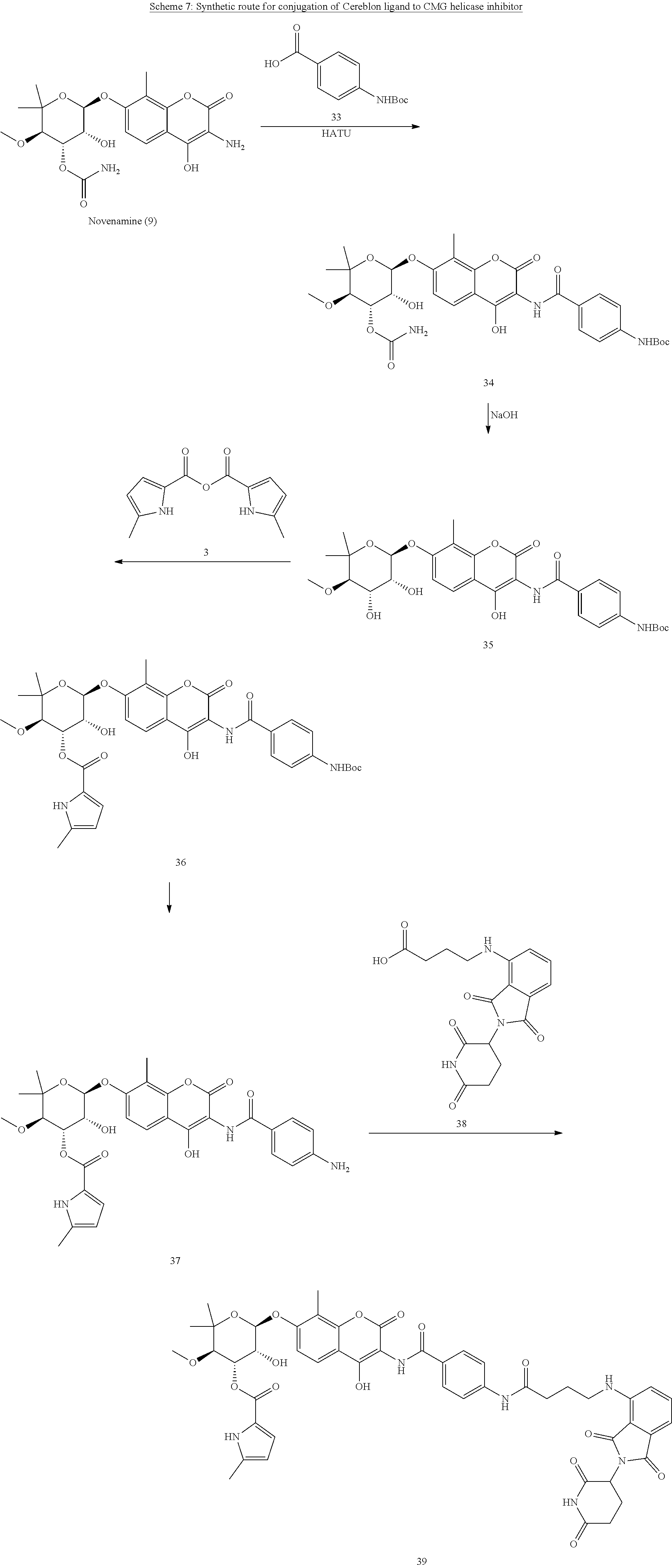

The synthesis is highly modular and can be adapted to access CMG helicase degraders incorporating different acyl groups (R⁶) at the C3′ position of the noviose sugar, variable benzamide spacers and a variety of E-3 ligase recruiting ligands of type 38. Some of these are shown in Schemes 8-10. Groups at R⁶ are introduced by acylation of intermediates of type 35 by use of activated carboxylic acids via appropriate coupling agent [e.g. HATU] or corresponding carboxylic anhydride (Scheme 8) which after Boc deprotection and coupling to the E-3 ligase recruiting ligands of type 38 (with variable linkers as defined in Scheme 11) provide the CMG PROTACs 40. The synthetic route to CMG helicase PROTACs 40 with different aminobenzamide groups is shown in Scheme 9. These are prepared by coupling the amine derivative 41 to an appropriate N-Boc protected carboxylic acid 42, followed by Boc removal and attachment of the linker-E3 ligase ligand group. Examples of terminal carboxylic acid linker-E3 ligase ligands [see, e.g., Han X-R, Chen L, Wei Y, Yu W, Chen Y, Zhang C, Jiao B, Shi T, Sun L, Zhang C, Xu Y, Lee M R, Luo Y, Plewe M B, Wang J. Discovery of Selective Small Molecule Degraders of BRAF-V600E. Journal of Medicinal Chemistry. 2020; 63(8):4069-80 and Li Y, Yang J, Aguilar A, McEachern D, Przybranowski S, Liu L, Yang C-Y, Wang M, Han X, Wang S. Discovery of MD-224 as a First-in-Class, Highly Potent, and Efficacious Proteolysis Targeting Chimera Murine Double Minute 2 Degrader Capable of Achieving Complete and Durable Tumor Regression. Journal of Medicinal Chemistry. 2019; 62(2):448-66] are also shown in Scheme 9, and can also include additional variable linkers as defined in Scheme 11. Alternatives to the coumarin group are shown in Scheme 10. The coumarin replacements 43 are introduced via the Mitsunobu coupling (3) with noviose derivative 5. Subsequent addition of the aminobenzamide and linker-E3 ligase ligand group provides the CMG PROTACs as shown in Scheme 10.

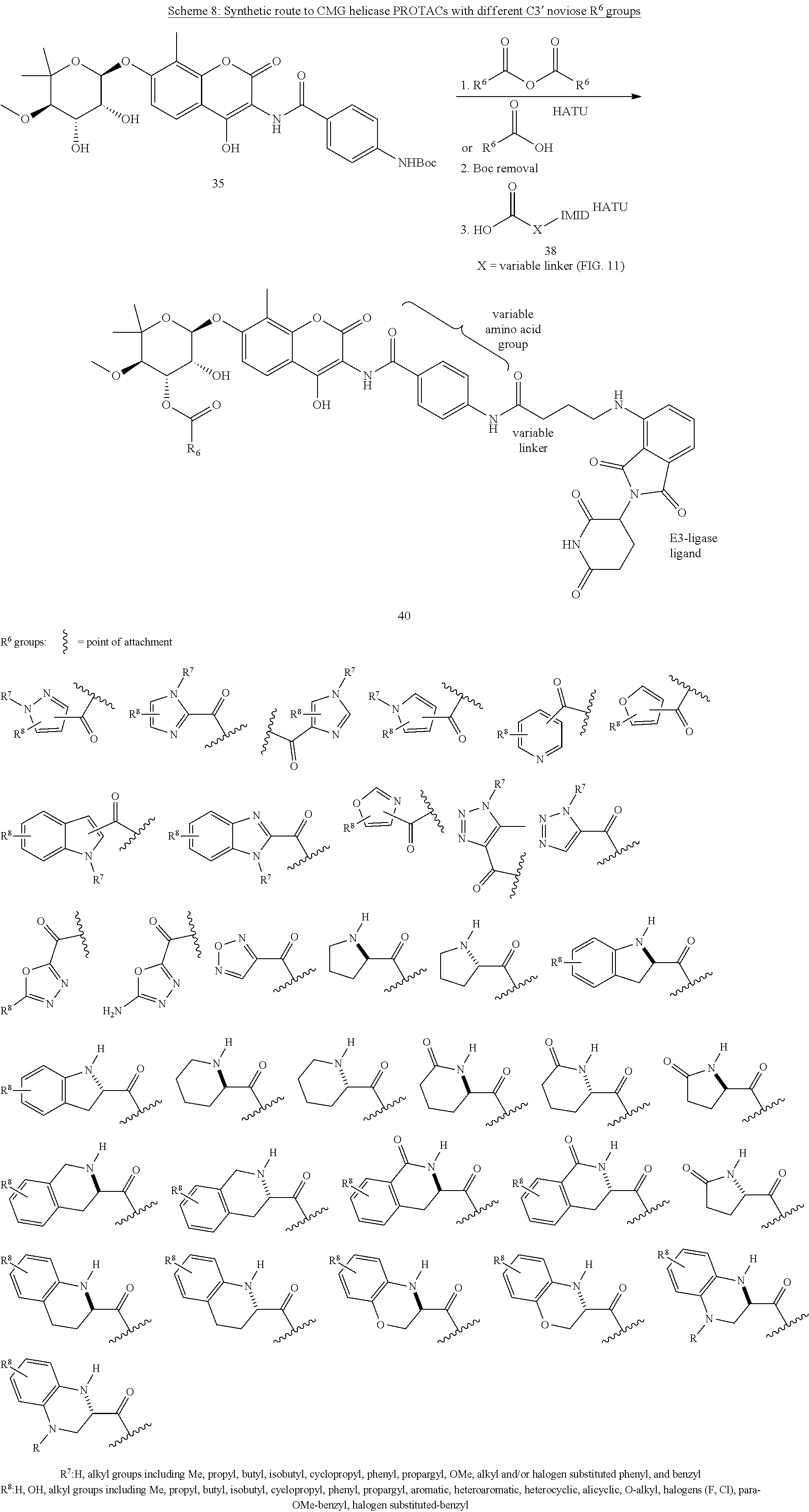

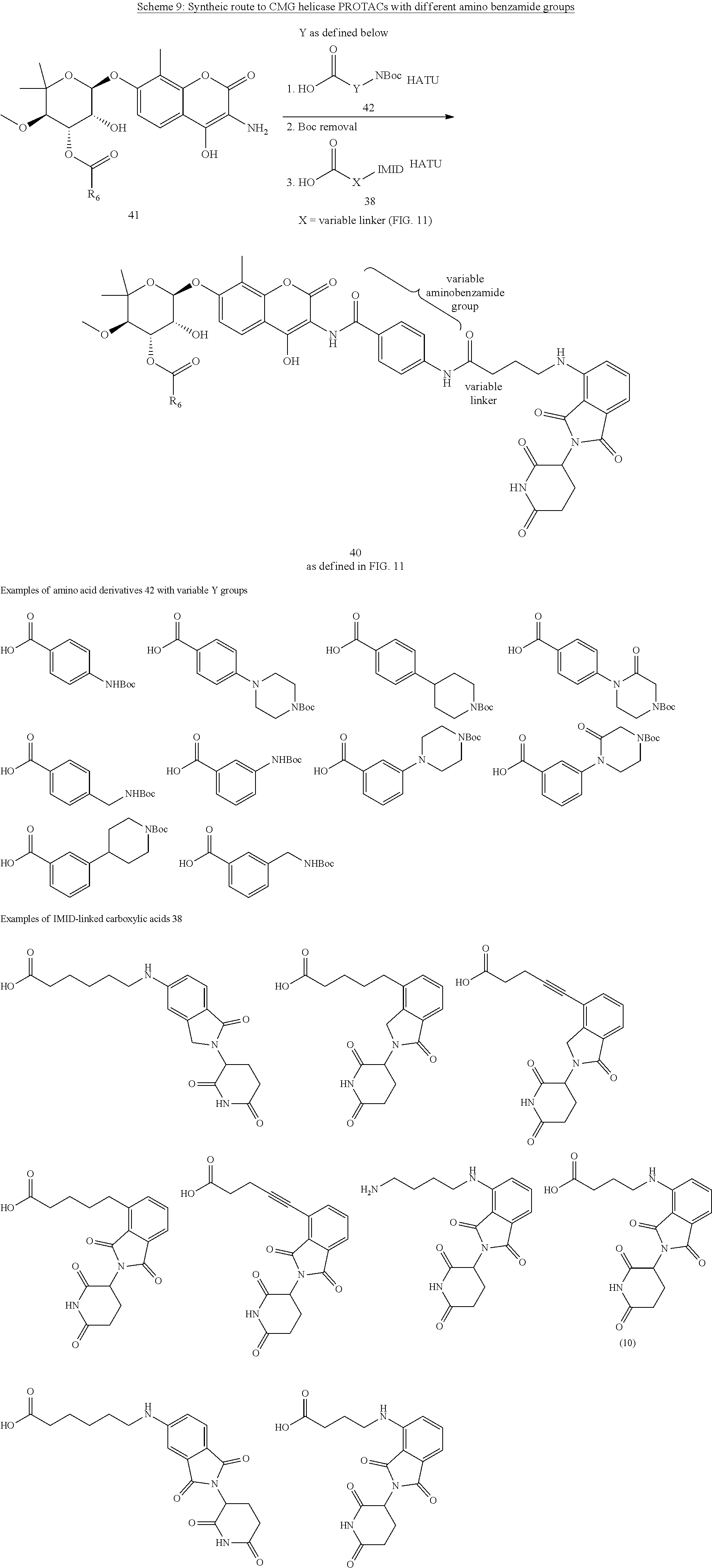

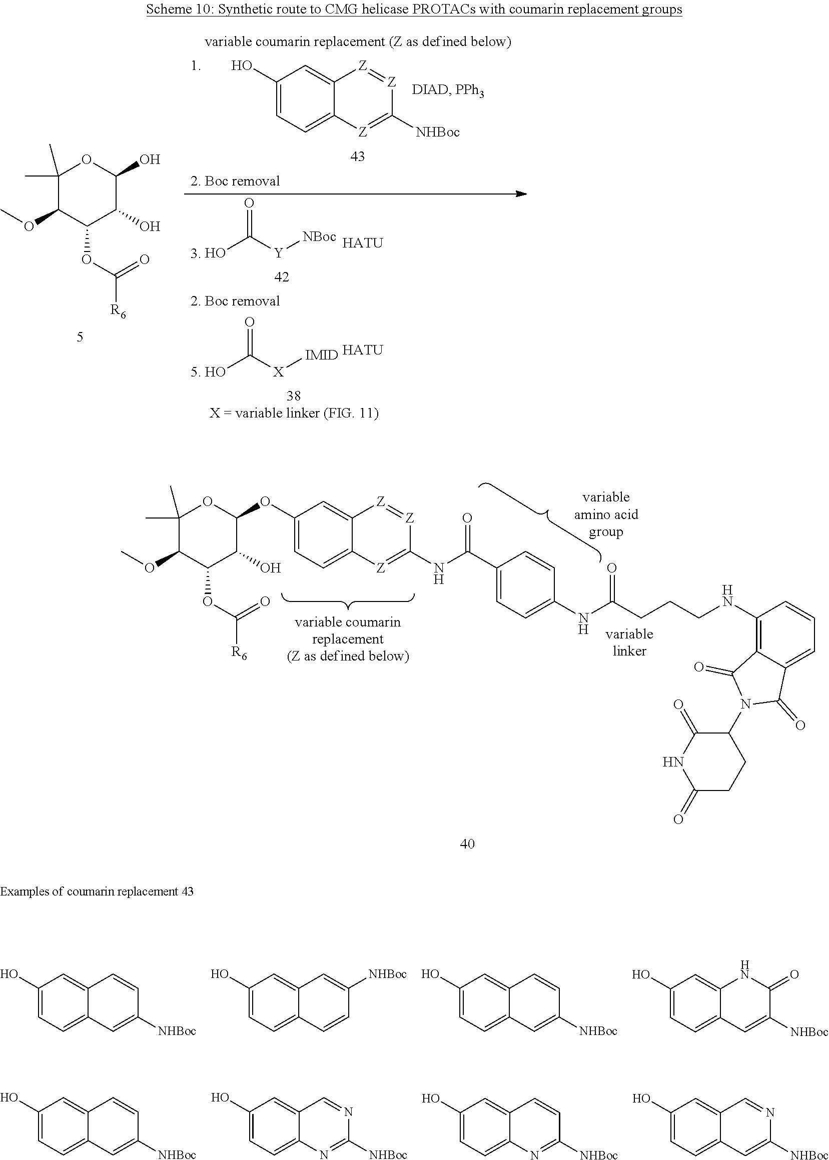

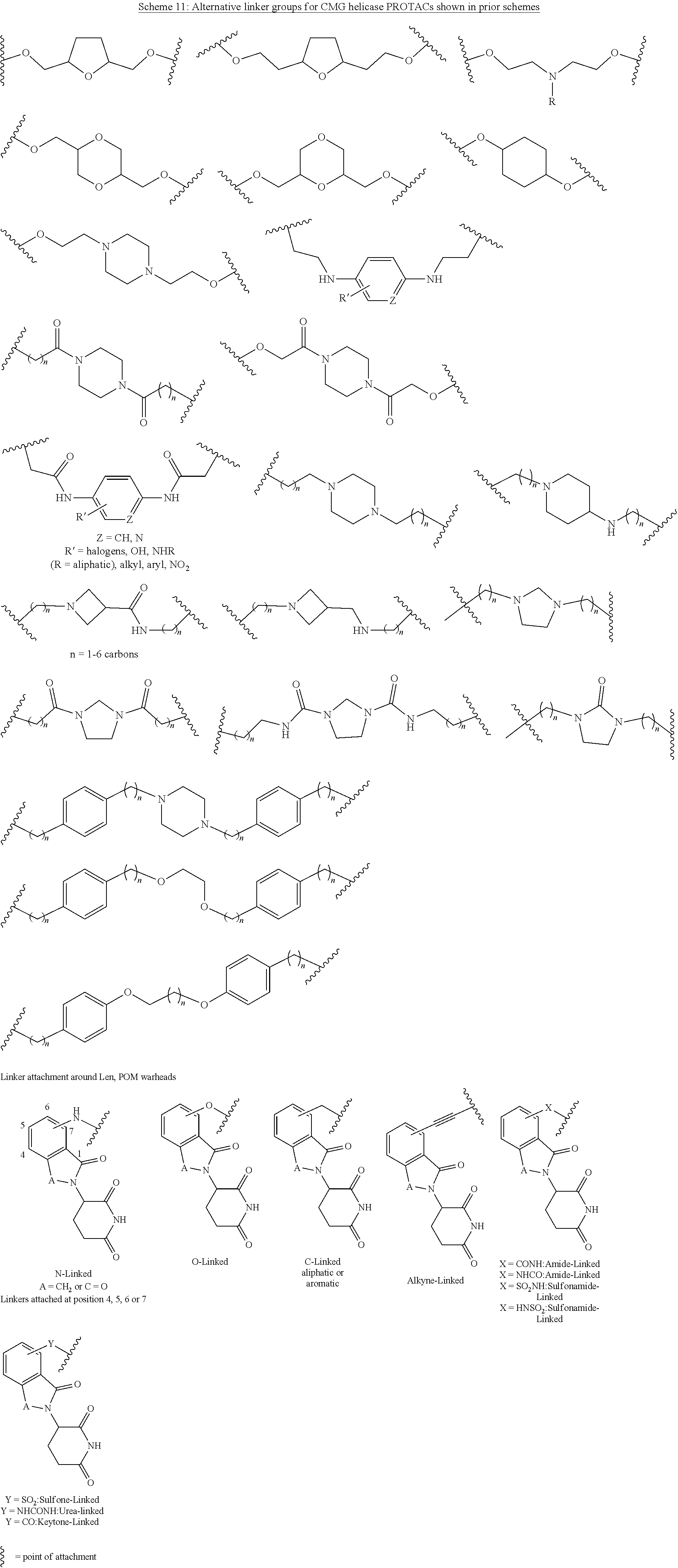

### Example 2. Identification of ATP-Competitive Human CMG Helicase Inhibitors for Cancer Intervention that Disrupt CMG-Replisome Function

The human CMG helicase (Cdc45-MCM-GINS) is a novel target for anti-cancer therapy due to tumor-specific weaknesses in CMG function induced by oncogenic changes and the need for CMG function during recovery from replicative stresses such as chemotherapy. Here, we developed an orthogonal biochemical screening approach and identified CMG inhibitors (CMGi) that inhibit ATPase and helicase activities in an ATP-competitive manner at low micromolar concentrations. Structure-activity information, in silico docking, and testing with synthetic chemical compounds indicate that CMGi require specific chemical elements and occupy ATP binding sites and channels within MCM subunits leading to the ATP clefts, which are likely used for ATP/ADP ingress or egress. CMGi are therefore also MCM complex inhibitors (MCMi). Biological testing shows that CMGi/MCMi inhibit cell growth and DNA replication using multiple molecular mechanisms distinct from other chemotherapy agents. CMGi/MCMi block helicase assembly steps that require ATP binding/hydrolysis by the MCM complex, specifically MCM ring assembly on DNA and GINS recruitment to DNA-loaded MCM hexamers. During S-phase, inhibition of MCM ATP binding/hydrolysis by CMGi/MCMi causes a ‘reverse allosteric’ dissociation of Cdc45/GINS from the CMG that destabilizes replisome components Ctf4, Mcm10, and DNA polymerase-a, -d, -e, resulting in DNA damage. CMGi/MCMi display selective toxicity toward multiple solid tumor cell types, targeting the CMG and inducing DNA damage and loss of viability at low micromolar concentrations. This new class of CMGi/MCMi provides a basis for small chemical development of CMG helicase-targeted anti-cancer compounds with distinct mechanisms of action.

### Introduction

The replicative CMG helicase is an emerging target for anti-cancer intervention due to exploitable vulnerabilities in cancer cells resulting from oncogene-driven mismanagement of CMG assembly and function (1). However, to date, no small chemical inhibitors of the human CMG helicase have been identified. The CMG is a multi-subunit enzyme that performs the primary DNA melting and unwinding steps within replisomes during DNA replication in eukaryotic cells (2). The CMG helicase is composed of Cdc45, a Mini-Chromosome Maintenance (MCM) heterohexameric ATPase core, and the GINS tetramer (Go-Ichi-Ni-San in Japanese for Sld5, Psf1, Psf2, and Psf3)(2-4). Assembly and activation of the CMG occur in a stepwise manner, with an excess of MCM hexamers loaded onto DNA during G1 phase (called Licensing (5,6)), followed by recruitment of Cdc45/GINS near the G1-S transition to a subset of these MCM hexamers (3,4,7,8). The dynamics of this MCM-CMG conversion process are important for maintaining genome stability in human cells (1). Unused MCM complexes act as reserves that are converted to CMG helicases during replicative stress to facilitate recovery of DNA replication (1,9-12). Reserve MCM complexes also modulate replication fork speeds to prevent DNA damage (13).

Oncogenic changes cause problems with CMG assembly and function (1). Cyclin E overexpression reduces the number of MCM hexamers that load onto DNA, resulting in reduced MCM reserves, loss of replication fork fidelity, and consequent DNA damage (1,14). Elevated Myc over-stimulates the conversion of MCM hexamers into CMG helicases, leading to DNA damage in genomic regions with excessive CMG activity due to increased replication fork density and reduction of unused MCM reserves (1,15-17). While these oncogenic events produce DNA damage that facilitates tumorigenesis and tumor heterogeneity, they also create a reduction in MCM/CMG functional fidelity in tumor cells that is likely exploitable with CMG inhibitors (1). In addition, inhibition of MCM/CMG reserves can selectively sensitize tumor cells to fork-stalling chemotherapy drugs (11,12), suggesting that CMG inhibitors will have the potential to overcome chemo-resistance in the management of cancer.

We report here the design of an orthogonal chemical screening approach and its use in the identification of the first ATP-competitive inhibitors of the ATPase and helicase activities of the human CMG enzyme (CMGi). These CMGi have drug-like features and are members of the aminocoumarin class of compounds (18), specifically clorobiocin and coumermycin-AI, while the closely related compound novobiocin is not effective at CMG inhibition. Consistent with this, biochemical analyses with synthetic compound derivatives show that CMGi require specific chemical elements for CMG inhibition. Modeling suggests CMGi can occupy multiple Mcm2-7 ATP-binding clefts and channels, the latter of which are likely used by ATP to access ATPase domains within the MCM ring. CMGi display distinct modes of action for cell growth inhibition and induction of DNA damage relative to other chemotherapy drugs, blocking MCM DNA binding and GINS recruitment during CMG assembly, and disrupting CMG-replisome co-structural integrity during S-phase. Tumor cells are selectively sensitive to low concentrations of CMGi that target the CMG helicase in vivo and in vitro, but not to novobiocin, strongly suggesting that differential effects of these aminocoumarins on tumor cells are derived from the sugar head group targeting CMG helicase inhibition. Importantly, these findings explain why aminocoumarins that function as CMGi had side effects in humans when tested as potential antibiotics in the distant past (18), and support the CMG helicase as a tumor-specific vulnerability and novel anti-cancer target. These CMGi can serve as unique probes to investigate ATPase-dependent CMG/MCM functions in human cells and can be used to inform the development of a new class of anti-cancer compounds that target and disrupt CMGs/replisomes.

### Results

### Identification of Human CMG Helicase Inhibitors (CMGi)

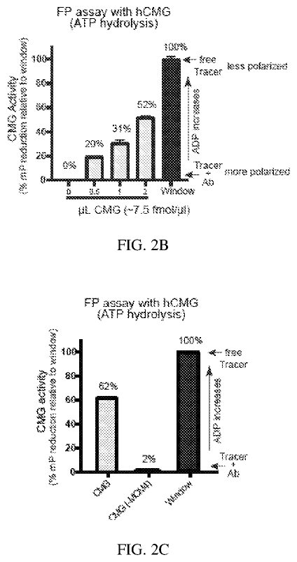

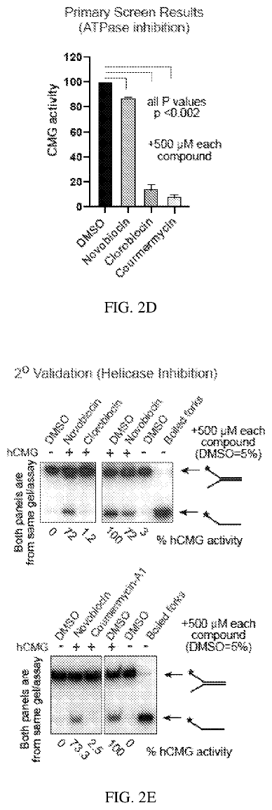

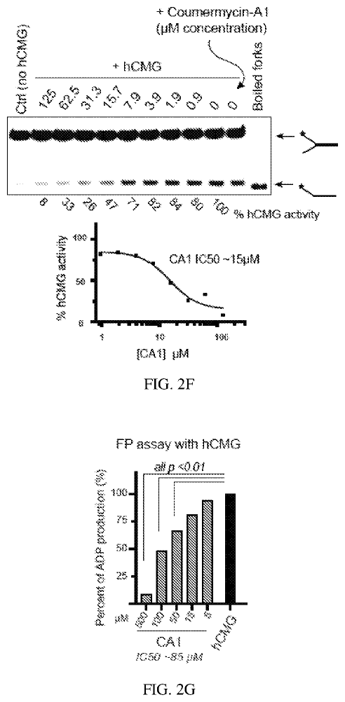

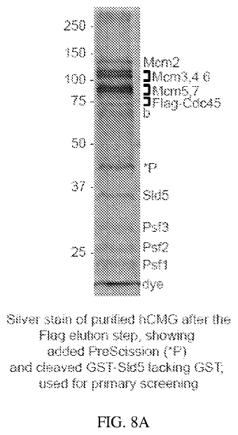

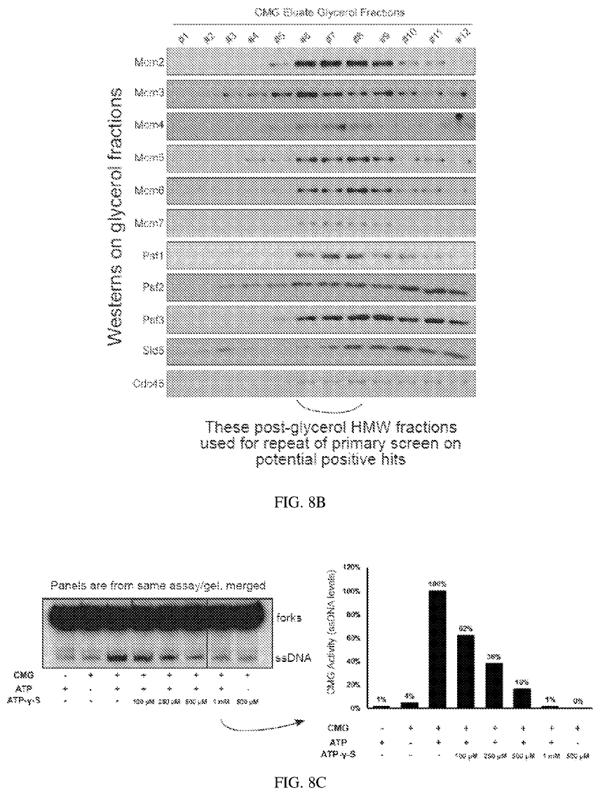

We developed a rigorous biochemical screening approach utilizing two rounds of primary screening with a commercially available ATP hydrolysis assay and a secondary orthogonal validation assay that measures DNA unwinding by the human CMG (hCMG; FIG. 2A). The hCMG helicase was purified using the established protocol of Hurwitz and colleagues in which all 11 hCMG subunits are co-expressed using baculoviral-based infections of insect cells followed by a multistep purification of the hCMG holoenzyme (FIG. 2A; detailed approach described below) (19,20). The quality of the hCMG enzyme obtained was verified by silver staining and immunoblotting, which showed that all 11 hCMG subunits were present at similar stoichiometries and purity compared to that obtained in previous studies (FIGS. 8A-8B) (19,20). The hCMG isolated is active in DNA fork-unwinding (helicase) assays and is dependent on the binding and hydrolysis of ATP as indicated by a dose-dependent suppression of fork-unwinding activity in the presence of slow-hydrolyzable ATP-γ-S (FIG. 8C). For fork-unwinding activity, the hCMG displays an ~Kₘ of 690 μM [ATP](see below), in close agreement with the Kₘ (625 μM [ATP]) for hCMG ATP hydrolysis activity described by others (19,20). The specific ATPase activity of the isolated hCMG also closely matches that obtained by Hurwitz and colleagues (see Methods)(19,20).

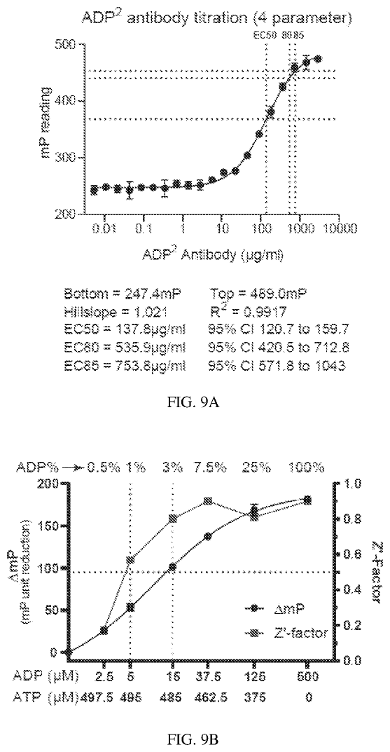

We optimized the ADP² Fluorescent-Polarization (FP) Transcreener Assay (BellBrook Labs, Madison, WI) for quantitative analysis of ADP production by the hCMG (see Methods and FIGS. 9A-9C). The assay relies on a patented anti-ADP² antibody that binds and polarizes an ADP-Tracer, changes to which due to competition with ADP produced by the hCMG are measurable in FP plate readers. This assay is highly sensitive and reliable (Z′>0.6; FIG. 9B), being able to quantify small changes in ADP production(21-24). We titrated purified hCMG into the assay, which produced a dose-dependent increase in ADP production (FIG. 2B). The hCMG concentration that produced an ~50% change in the assay window was used for screening. Because significant quantities of hCMG are required for screening, we performed primary chemical library screening with hCMG purified through the Flag enrichment step, and a repeat of primary screening with positive hits on a higher purity (but lower yield) hCMG after glycerol fractionation (FIG. 2A). The hCMG obtained after the Flag enrichment step is active in the primary assay and is dependent on the presence of intact hCMG helicase, as failure to express Mcm4 yields preparations devoid of ATPase activity (FIG. 2C). The latter indicates that a contaminating ATPase from insect cells is not present in our hCMG preparations during our screening.

We used the National Institutes of Health (NIH) Diversity Set VI library at 1 mM concentrations for primary screening, and repeated screening of positive hits at 500 μM. Fewer than 3% of compounds were capable of partial or complete hCMG inhibition. One compound, clorobiocin, drew attention due to its drug-like features and effective nature of hCMG inhibition (FIG. 2D). Clorobiocin did not interfere with far-red UV light readings or assay reagents. Clorobiocin is an aminocoumarin derived from Streptomyces roseochromogenes and is related to two similar chemicals, novobiocin and coumermycin-A1 (18). It was difficult to continue working with clorobiocin, as we needed fresh dry chemical powder for further validation work, the NIH had none available, and we could not find a commercial source. However, coumermycin-A1 (CAI) and novobiocin were commercially available, and we tested both for their ability to inhibit the hCMG. CA1 was found to be a potent inhibitor of ATP hydrolysis by the hCMG, while novobiocin had very little inhibitory effect on the hCMG (FIG. 2D).

Validation with our secondary strand-displacement (helicase) assay showed that clorobiocin and CA1, but not novobiocin, were effective hCMG helicase inhibitors at 500 μM concentrations (FIG. 2E). The in vitro IC₅₀ of CA1 for hCMG helicase inhibition was determined to be ~15 μM (FIG. 2F), which closely matches the IC₅₀ of CA1 for reducing viability of human cells (see below). Using the FP assay and an ADP/ATP standard curve comparison, the IC₅₀ for hCMG ATP hydrolysis inhibition is ~85 μM (FIG. 2G). The hCMG has six distinct ATPase clefts and it is likely that CA1 does not target all of them with the same efficiency (see below). It is therefore possible CA1 targets a cleft(s) necessary for helicase activity at higher affinity, but more CAI is necessary to inhibit remaining ATP sites.

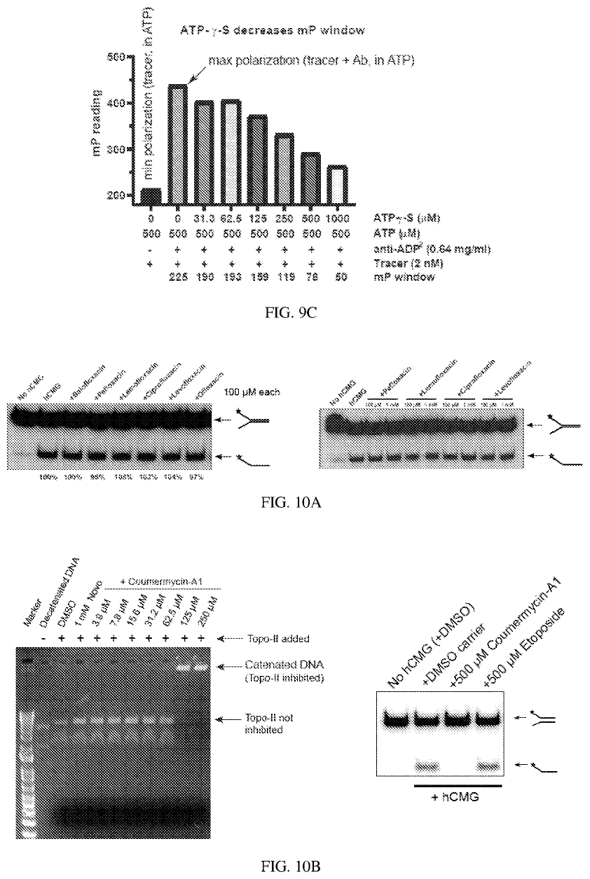

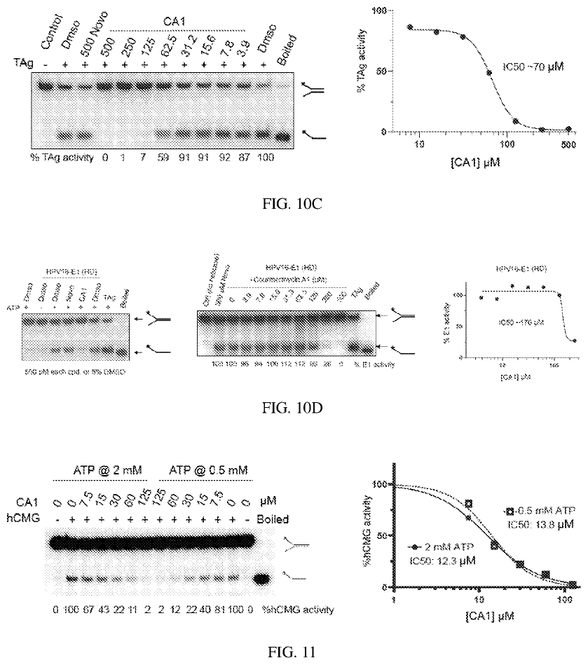

Using yeast MCM complexes as the basis for screening, it has been suggested that ciprofloxacin might be an inhibitor of the human replicative helicase (25,26). However, high concentrations of ciprofloxacin or other quinolones do not inhibit the purified hCMG helicase (FIG. 10A). CA1 is known to inhibit the bacterial type-2 topoisomerase, gyrase(18). However, relative to that required to block CMG activity, inhibition of human Topo-II in decatenation assays in vitro requires ~10-fold higher CA1 concentration (FIG. 10B). To assess selectivity of CA1 toward the CMG helicase, we tested whether CA1 could inhibit two related mammalian hexameric ring helicases with significant structural similarity to the CMG, the Large-T antigen (TAg) helicase from SV40 virus, and E1 helicases from human papilloma viruses (HPV). Purified TAg helicase and E1 helicases from HPV16 and HPV18 are sensitive to CA1, but require higher CA1 concentrations (TAg IC₅₀~70 μM; HPV16-E1 IC₅₀~170 μM; FIGS. 10C-10D). These results indicate that CA1 displays in vitro selectivity toward the CMG helicase at low concentrations. While it is possible CA1 might affect additional helicases (or other ATPases), the remainder of this report will focus on understanding the chemical warhead and mechanisms for CAI as a CMG inhibitor, as CA1 and clorobiocin represent the first biochemically-validated small chemical compounds that effectively inhibit ATPase and helicase activities of the hCMG (defined as CMGi).

Coumermycin-A1 is an ATP-Competitive Inhibitor of hCMG Activity

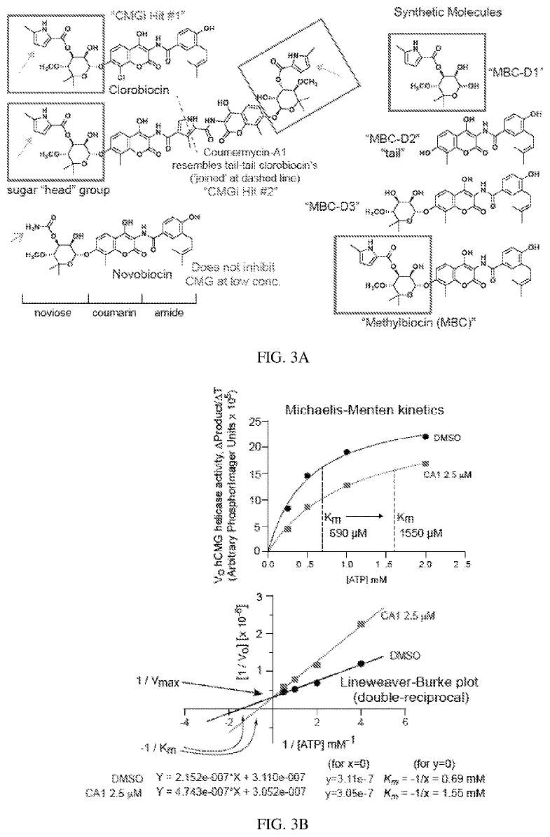

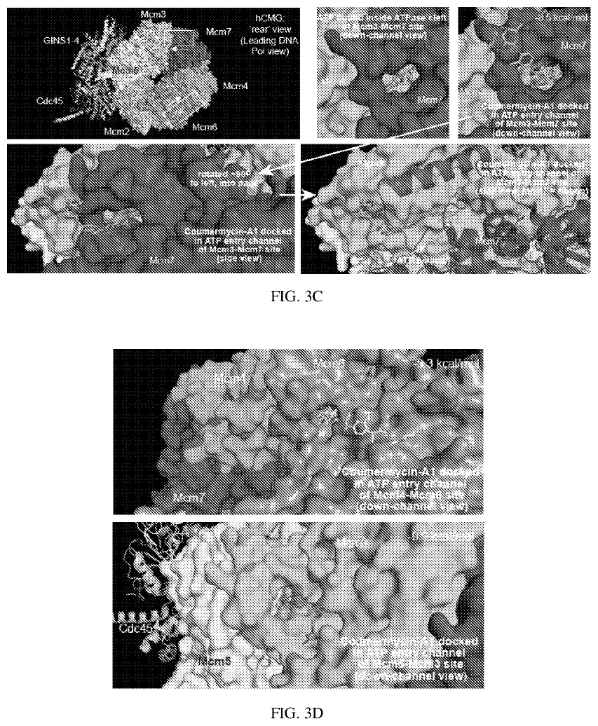

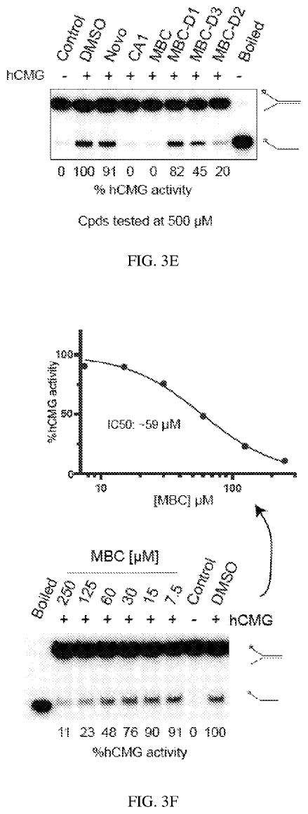

We determined the mechanism of hCMG inhibition by CA1. The three aminocoumarins are comprised structurally of a noviose sugar “head” group joined to a coumarin group, and an amide group in two of the molecules (FIG. 3A; coumarin-amide domain referred to here as the ‘tail’ of the molecule) (18). Clorobiocin and CA1 contain 2-methylpyrrole ester modifications to the sugar (FIG. 3A, orange arrows) whereas novobiocin has a primary carbamate modification (FIG. 3A, blue open arrow). CA1 resembles a tail-tail dimer of clorobiocin, but replaces the chlorine with a methyl. Since novobiocin has little inhibitory effect on the hCMG in the biochemical assays (only at high concentrations), and largely differs from the other compounds in the modification of its sugar, this structure-activity relationship (SAR) indicates that the sugar head groups of clorobiocin and CA1 (FIG. 3A, purple boxes) provide chemical specificity in mediating inhibition of the hCMG.

Biochemical and co-crystallographic data assessing how these aminocoumarins inhibit gyrase (a type-II topoisomerase) provide information on how CA1 can inhibit the hCMG (18,27-29). Aminocoumarins inhibit ATP binding and hydrolysis of gyrase using a competitive mechanism, inserting the sugar head groups through a channel/groove into the ATPase cleft of the GyrB subunit, with the sugar situated in the region where the adenosine and ribose of ATP normally interact (18,27). CA1 interacts with two GyrB ATPase domains at the same time using this mechanism (18,27). We reasoned that CAI might likewise inhibit hCMG ATPase and helicase activities by direct competition with ATP binding and hydrolysis. We performed hCMG helicase assays to determine the mode of hCMG inhibition by CA1 in increasing ATP concentrations (FIG. 3B). Michaelis-Menten kinetics and Lineweaver-Burke (double-reciprocal plot) analyses showed that CA1 inhibits hCMG helicase activity using a classic ATP-competitive mechanism, resulting in a significant increase in the Kₘ, for [ATP](690 μM without inhibitor to 1550 μM with CA1), without a change in the Vₘₐₓ of the hCMG. This significant shift in Kₘ explains why low micromolar concentrations of CA1 are capable of inhibiting the hCMG in the presence of high ATP concentrations, as occurs in a cellular environment (FIGS. 3B and 11).

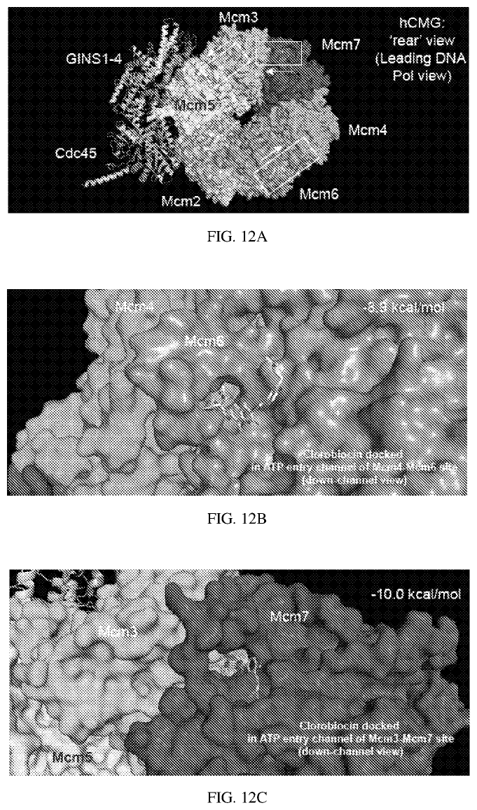

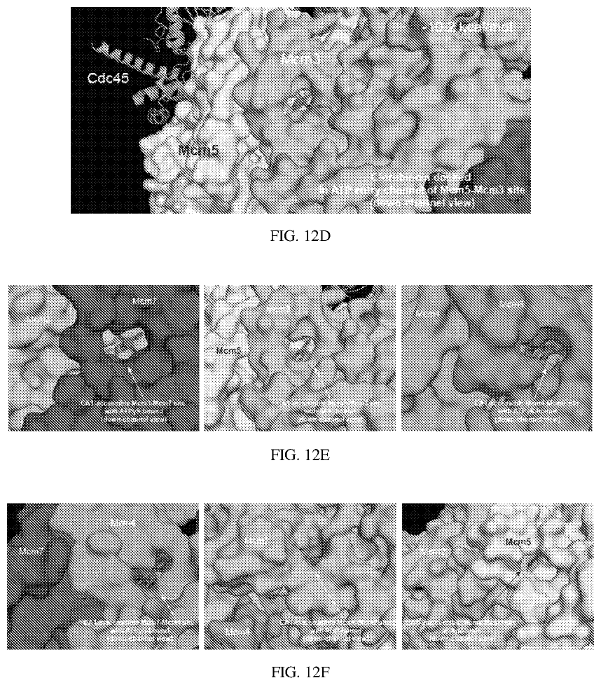

We next used in silico docking of CA1 and clorobiocin to model how these compounds can interact with the ATPase domains of the hCMG to competitively block ATP binding and hydrolysis. The hCMG has six biochemically distinct ATPase domains formed between adjacent MCM subunit pairs (4,30-32), and the cryo-EM structure of the hCMG has been determined (33). Docking software places CA1 and clorobiocin into channels leading to the ATPase clefts of three MCM ATPase domains with similar binding energies, notably clefts for Mcm3-Mcm7, Mcm4-Mcm6, and Mcm5-Mcm3 (FIGS. 3C-3D, CA1 docking; FIGS. 12A-12D, clorobiocin docking). Consistent with co-crystallographic data for aminocoumarin-gyrase interactions (18,27-29), the sugar head groups are inserted into the ATP binding sites where adenosine and ribose from ATP are normally situated (‘sugar-first’ direction), while the coumarin group occupies channels leading to the ATP binding sites. CA1 can be docked in either direction in these MCM ATPase clefts/channels due to its symmetry. It is quite possible that these channels are used by ATP or ADP for ingress and/or egress during enzyme function, suggesting that clorobiocin and CA1 act like a ‘cork in a wine bottle’ to block ATP movement through the channels, consistent with their ATP-competitive mode of hCMG inhibition. Three MCM ATPase domains were not capable of in silico docking for either compound, specifically the clefts for Mcm7-Mcm4, Mcm6-Mcm2, and Mcm2-Mcm5. In the existing hCMG structure (33) the channels leading to these ATPase clefts are narrow relative to the MCM sites that can be docked, suggesting a steric hindrance to inhibitor binding (FIGS. 12E-12F). The cryo-EM structure of the hCMG is in one enzymatic state, and it remains possible that these sites might also be subject to inhibition by clorobiocin or CA1 under different enzymatic states when these channels might be accessible.

We prepared several compounds to support the SAR and modeling results obtained with the CMGi identified in our screen. A compound referred to herein as methylbiocin (MBC) was synthesized that is identical to clorobiocin, except for replacement of the chlorine with a methyl, and is thus half of the CA1 molecule (FIG. 3A; synthesis of all compounds and NMR purities described below)(34-36). Derivatives of MBC were also synthesized: a noviose sugar-pyrrole compound (MBC-D1), a coumarin-benzamide “tail” compound (MBC-D2), and a compound comprised of the coumarin-benzamide tail with the noviose sugar (MBC-D3). All compounds were initially tested at 500 μM concentrations to assess for inhibition of hCMG helicase activity (FIG. 3F). The MBC compound and CA1 both potently inhibit the hCMG, while novobiocin and the sugar-pyrrole compound (MBC-D1) show only a small level of inhibition. Interestingly, the tail compound (MBC-D2) is somewhat effective at inhibiting the hCMG, but this inhibition of the hCMG is diminished when the noviose sugar is added (FIG. 3E, MBC-D3). The IC₅₀ for MBC was ~59 μM, which is lower than that for CA1 (FIG. 3F). The IC₅₀ for the other derivatives was not determined since these compounds elicited only partial hCMG inhibition at high micromolar concentrations. These SAR results demonstrate that inhibition of hCMG activity is derived from a chemical warhead comprised of the coumarin moiety (“tail”) linked to the noviose-pyrrole (sugar “head”), which modeling suggests co-occupy the channel and ATP binding site(s) to competitively limit ATPase function. CA1 contains two of these MBC warheads, which may partly explain why CA1 is a better inhibitor than MBC.

At present we do not know if certain MCM ATPase clefts display preferences for inhibitor binding over other clefts, particularly in cells, as determining this is technologically challenging for an enzyme comprised of six ATPase domains that are not easily separated for analysis. Going forward, we assess hCMG ATPase/helicase inhibition and chemical biology in cells with commercially available CA1 from a holo-enzyme perspective (ATP-competitive effects on all clefts combined) rather than a particular ATPase cleft.

### CMGi Inhibit MCM/Cdt1 Assembly on Chromatin

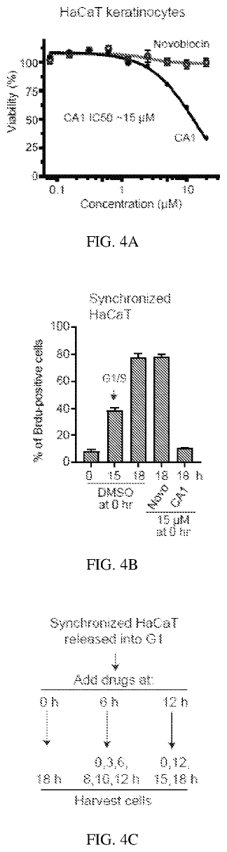

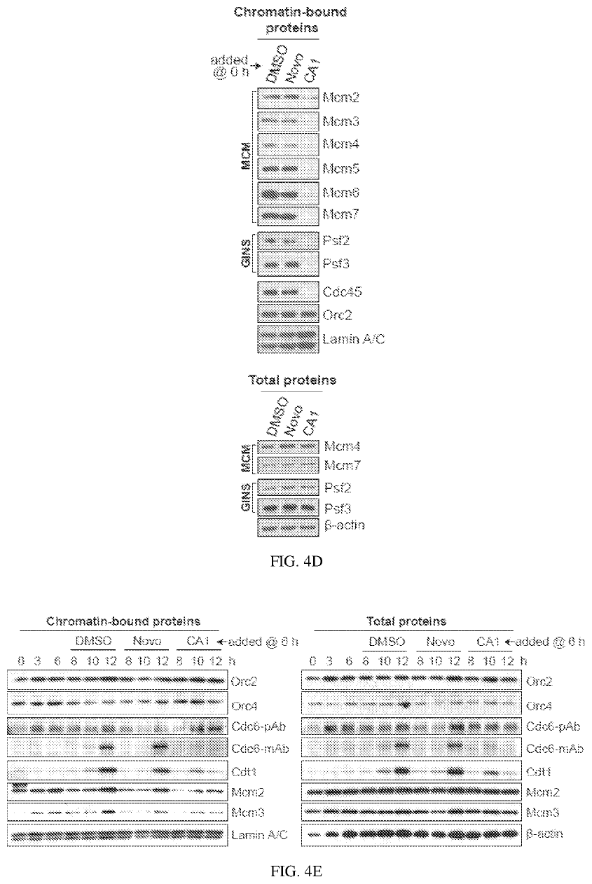

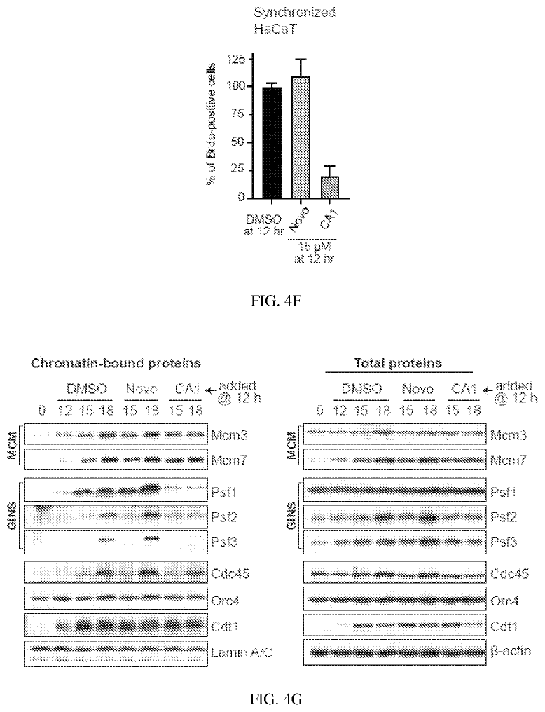

We used the immortalized, non-tumor derived human keratinocyte HaCaT cell line to assess effects of CMGi on growth, DNA replication, and CMG helicase assembly and function. A cell viability analysis found that CA1 reduces viability with an IC₅₀ of ~15 μM (FIG. 4A). Since novobiocin has little effect on viability, and this concentration of CA1 aligns with that required to inhibit in vitro hCMG helicase activity, these results suggest that the hCMG is likely a major target of CA1 in human cells at these low concentrations. As shown previously(37), HaCaT cells are efficiently synchronized in a quiescent state by serum deprivation for 2 days and released into the cell cycle to study G1 and S-phase events after re-adding serum. Using BrdU labeling and immunofluorescent measurements to assess DNA replication in synchronized HaCaT cells (see Methods), the start of G1 occurs at time 0 (serum re-addition), the G1/S transition occurs at ~15 hrs post release into the cell cycle (when ~50% of population is labeled), and the peak of S-phase occurs during the 18-20 hr window (FIG. 4B) (37). CA1 inhibits DNA replication when added to HaCaT cells in early G1 (at time 0), while novobiocin has little effect (FIG. 4B), consistent with assembly and activity of hCMG complexes being required for G1 progression into S-phase.

Studies of MCM assembly using yeast in vitro models have suggested that ATP binding and hydrolysis by most MCM ATPase clefts are required for efficient Mcm2-7 ring loading onto DNA (31,32). We asked whether CMGi/CA1 could block chromatin/DNA binding of human MCM complexes in vivo due to a dependency on ATP utilization. Synchronized HaCaT cells were treated with CA1, novobiocin, or DMSO carrier in early-G1 (at release, 0 hrs), middle-G1 (6 hrs), or late-G1 (12 hrs) to assess effects of CMGi on different stages of MCM assembly (FIG. 4C). MCM loading onto chromatin in human cells is significantly inhibited by early-G1 CA1 treatment (FIG. 4D). GINS and Cdc45 loading onto chromatin is consequently blocked, while Orc2 chromatin binding is not affected. This suggests that the ORC complex, which contains ATPase domains required for its DNA-binding and roles in MCM loading, is not itself a target of CA1.

Some MCM complexes are already loaded onto chromatin between early and middle G1 (3-10 hrs after release), but an increase in MCM loading occurs around 12 hrs as cells approach G1/S (FIG. 4E). While exposure of cells to CA1 at 6 hrs does not affect MCMs already loaded, the increase in MCM loading at 12 hrs is inhibited by CA1 but not by novobiocin. Cdt1 also loads onto chromatin at higher levels when MCM loading increases. However, CA1 blocks this Cdt1 loading and promotes loss of total Cdt1 (FIG. 4E). Taken together with the previous experiment, these results indicate that CMGi inhibit an early step in the MCM loading process in human cells that requires efficient ATP binding and/or hydrolysis by Mcm2-7, but once loaded, MCMs are resistant to CMGi/CAI. The results also suggest that Cdt1 is sensitive to Mcm2-7 ATPase inhibition in human cells, consistent with yeast studies showing that MCM-Cdt1 interactions are adversely affected by defective Mcm2-7 ATPase sites (31,32).

Orc2 and Orc4 affinity for chromatin is not affected by CA1 exposure in middle G1 (FIG. 4E), again suggesting that the ATPases of ORC are not a target of CA1. Cdc6 protein is not affected in chromatin association by CA1 when measured using a polyclonal antibody (FIG. 4E). In contrast, analysis with a monoclonal antibody to Cdc6 suggests that one form of Cdc6 increases on chromatin in parallel with MCM elevation and is sensitive to CA1. Cdc6 contains an ATPase domain that is not required for MCM assembly on DNA, but instead for removal of improperly loaded MCMs (31,32). While we cannot rule out the possibility that the ATPase site of Cdc6 may be affected by CA1, these prior studies suggest that it is unlikely that this would contribute to the inhibition of MCM assembly we have observed in the presence of CA1.

### GINS Recruitment to DNA-Loaded MCM Complexes is Inhibited by CMGi

Synchronized HaCaT cells treated with CA1 in late-G1 (treated at 12 hrs), but not novobiocin, fail to undergo DNA replication (FIG. 4F). Near the G1/S transition (15-18 hrs) there is additional MCM loading onto chromatin, which coincides with GINS and Cdc45 being recruited to loaded MCM hexamers on chromatin (FIG. 4G). Treatment with CA1 during this late-G1 period has only a small effect, if any, on the remainder of MCM loading and does not affect Cdt1 dynamics, suggesting that Mcm2-7 ATPase functions in MCM/Cdt1 loading are no longer required at this later time. However, while Cdc45 is not appreciably affected, CA1 inhibits GINS recruitment to DNA-loaded MCM hexamers (FIG. 4G). Such results are consistent with yeast in vitro studies showing that certain ATPase sites of the Mcm2-7 ring are required for GINS binding(32). However, our results differ somewhat from another yeast study showing that GINS and Cdc45 are both dependent on ATP binding to the Mcm2-7 ring(38). Reasons for this difference may be that CA1 is less efficient at binding a particular MCM ATPase cleft involved in Cdc45 recruitment in human cells, or that the Cdc45 extraction conditions vary between experimental approaches.

### Multiple Kinases are not Targeted by CMGi in Human Cells

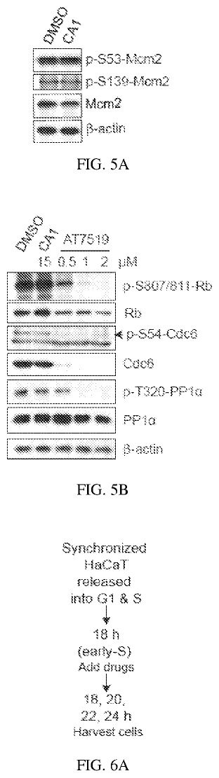

There are other enzymes with ATPase domains that function in MCM/CMG assembly, including Dbf4-Cdc7 (DDK) and Cdk2 (2). We asked whether CA1 affected these and other enzymes in human cells. DDK phosphorylates two sites in Mcm2 (S53 and S139) to facilitate Cdc45 recruitment (39), both of which show no phosphorylation changes after extended exposure to CA1 (FIG. 5A). This agrees with our observation that Cdc45 is recruited to DNA-loaded MCM hexamers (FIG. 4G) and indicates that DDK is not a target of CA1. A pan-Cdk inhibitor (AT7519) that efficiently targets Cdk1 (Cdc2), Cdk2, Cdk3, Cdk4, Cdk6, and Cdk9 blocks phosphorylation of Cdk2 targets, including Rb and Cdc6 (S54P) (40) (41), and the Cdk1 target PP1a (42). However, extended exposure to CA1 has no effect on these substrates (FIG. 5B). We conclude that CA1 does not target these kinases required for MCM/CMG assembly. Although we cannot exclude the possibility that other unknown CA1 targets exist in cells, particularly at higher concentrations, these results support that the effects of CAI on MCM/CMG dynamics in cells are due primarily to targeting of the Mcm2-7 ATPases.

### CMGi Disrupt Helicase and Replisome Co-Structural Integrity

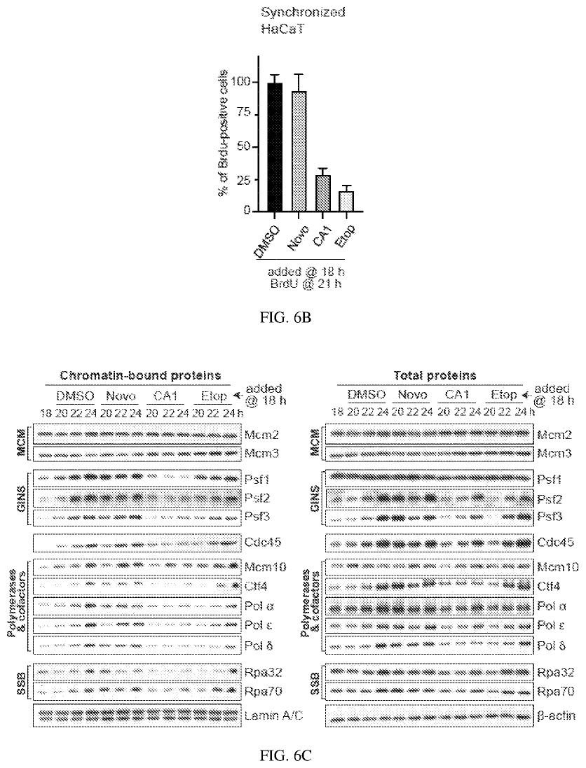

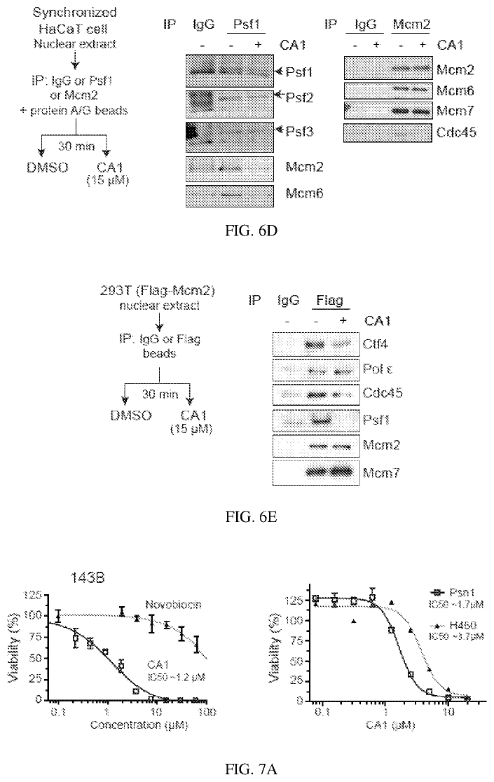

We determined how CMGi exposure affected the dynamics of hCMG and replisome structure during S-phase. Synchronized HaCaT cells were treated with CA1, novobiocin, or DMSO once cells reached early S-phase (18 hrs) and immunoblots were performed assessing chromatin-bound and total protein components of the replication machinery (FIG. 6A). Etoposide has no effect on hCMG helicase activity in vitro (FIG. 10A), and was included to compare how inhibition of Topo-II affected replication dynamics. Treatment with CA1 and etoposide effectively suppressed DNA replication, while novobiocin/DMSO did not (FIG. 6B), confirming that hCMG and Topo-II activities are required for ongoing DNA replication. MCM association with chromatin/DNA was not affected by any compounds (FIG. 6C, left). However, GINS and Cdc45 chromatin association was notably suppressed by CA1, and not by novobiocin or etoposide. Total protein levels were slightly affected for Psf3 and Cdc45, but not for other subunits (FIG. 6C, right). We conclude from these results that DNA topological issues and DNA replication arrest due to Topo-11 inhibition do not disrupt GINS/Cdc45 interactions with hCMG helicases. However, CMGi inhibition of ATP binding and/or hydrolysis by the Mcm2-7 ATPases results in GINS and Cdc45 dissociation from hCMG helicases during ongoing DNA replication. Two related interpretations are possible. CA1 could block GINS/Cdc45 from being recruited or cause dissociation after recruitment (latter tested below.)

Structural studies have shown that components of the human replisome interact directly with the hCMG helicase, mediated in part through GINS and Cdc45 (43,44). We asked if CMGi-induced loss of Cdc45 and GINS from hCMGs resulted in disruption of replisomes in human cells. Treatment of S-phase cells with CA1, but not other compounds, caused a loss of DNA polymerases-a, -d, and -e from chromatin (FIG. 6C). Factors such as Ctf4 and Mcm10, which interact with the hCMG and facilitate DNA polymerase-a function on the lagging strand(43,44) are also reduced on chromatin by CA1. Consistent with hCMG and replisome disruption, RPA (single-stranded binding protein) is also reduced on chromatin. Intriguingly, exposure to etoposide, which stops DNA replication, does not significantly diminish replisome components or RPA on chromatin, except for a small change to DNA polymerases-a and -d (FIG. 6C). We conclude that, while inhibition of Topo-II (and DNA replication) does not have a significant negative effect on replisome integrity, the structural co-integrity of replisomes and hCMGs is dependent on ATP binding and/or hydrolysis by the Mcm2-7 ATPases during ongoing DNA replication in human cells and is disrupted by CMGi.

We next determined if CMGi treatment of partially purified hCMG helicases and replisomes from human cells were sensitive to CA1 after such complexes have formed. Nuclear extracts were prepared from HaCaT cells enriched in S-phase and subjected to immunoprecipitation using antibodies to Psf1 or Mcm2. Immunoprecipitated complexes were treated directly with CA1 or DMSO, followed by immunoblotting for associated proteins (FIG. 6D). Psf1 associates with Psf2, Psf3, Mcm2, and Mcm6, indicating that hCMG helicases were extracted from cells (FIG. 6D, middle). We could not examine Cdc45 in this experiment due to signal interference with IgG on immunoblots. Treatment with CA1 did not disrupt Psf1-3 interactions, indicating that the GINS complex itself is not abrogated by CA1. However, Mcm2 and Mcm6 interactions with GINS are abolished by CA1. Mcm2 associates with Mcm6, Mcm7, and Cdc45, and CA1 treatment causes Cdc45 to dissociate from MCMs but does not disrupt MCM complexes (FIG. 6D, right). We performed a similar experiment using a different human cell line (HEK-293T) expressing ectopic Flag-Mcm2 (FIG. 6E). Flag-Mcm2 interacts with endogenous Mcm7, Psf1, Cdc45, DNA polymerase-e, and Ctf4 (FIG. 6E, right), indicating that Flag-Mcm2 forms complexes with hCMG and replisome components in human cells. CA1 does not disrupt MCM interactions but displaces Psf1, Cdc45, and Ctf4 from Flag-Mcm2. Interestingly, CMGi does not disrupt DNA polymerase-e binding to Flag-Mcm2, suggesting that differences exist between replisome-hCMG interactions in cells and in vitro. A possible explanation is that in vivo other factors may contribute to replisome disassembly, such as the ubiquitin ligase CUL2(LRR1), which contributes to disassembly (45). These experiments demonstrate that CMGi disrupt the structural co-integrity of hCMG and replisome components after complexes have formed. MCM hexamers are not disrupted by CMGi, consistent with yeast studies using MCM ATPase mutants (31,32). However, interactions of GINS, Cdc45, DNA polymerases, and co-factors with hCMG helicases depend on functional MCM ATPase domains during S-phase that are inhibited by CMGi exposure.

### Tumor Cells Are Selectively Sensitive to CMGi-Induced DNA Damage/Apoptosis

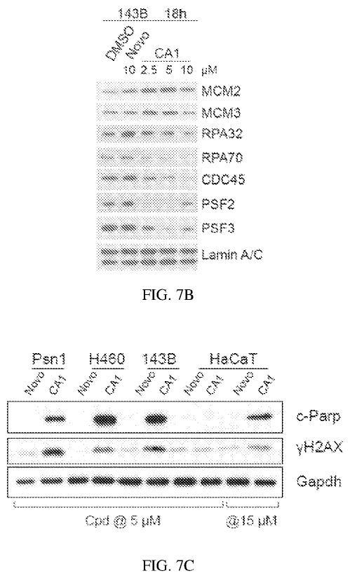

Oncogenic signals together with loss of tumor suppressor protein function, such as p53, elicit changes in MCM/CMG assembly/activation, or the need for CMG function in DNA damage or replicative stress (RS) recovery, that predict CMG vulnerabilities may exist in some solid tumor cells (1). We assessed the CMGi/CA1 effects on three tumor lines from malignancies that currently have limited treatment options or few effective targeted therapies (Psn1 pancreatic ductal adenocarcinoma; H460 non-small cell lung carcinoma; 143B osteosarcoma/OS) to that of non-tumor HaCaT cells. In viability assays, all three tumor lines are 4-10 times more sensitive to CA1 exposure relative to HaCaT, with IC₅₀ estimates of 1-4 μM (FIG. 7A, compare to FIG. 4A). Novobiocin has little effect until higher concentrations are tested. Using asynchronous 143B OS cells as an example, low micromolar CA1 targets the CMG in tumor cells, reducing the chromatin-bound levels of Cdc45, Psf1, and Psf2, while also reducing RPA levels in a dose-dependent manner (FIG. 7B). At higher CA1 doses MCM levels start to diminish. Exposure of all three tumor lines and HaCaT cells to 5 μM CA1 shows a tumor cell-specific increase in DNA damage signals (gamma-H2AX surrogate) and Parp cleavage indicative of apoptosis. Increasing the CA1 dose to 15 μM shows that HaCaT cells will eventually incur DNA damage and Parp cleavage at higher doses. Importantly, these results suggest that the CMG is a tumor-selective vulnerability in these tumor cells and that a therapeutic window likely exists for targeting the hCMG (or MCM complex) in future anti-cancer clinical regimens.

### Discussion

We have identified the first small chemical compounds capable of inhibiting ATPase and helicase functions of the human replicative CMG helicase, as an important advance toward the development of effective CMGi with anti-cancer clinical applications. Common targets in anti-cancer chemotherapy regimens often include the DNA replication and repair machinery that functions at replication forks or damaged DNA sites. Clearly, the CMG helicase represents another such target that has yet to be drugged, and we show here distinct mechanisms of action used by CMGi to abrogate DNA replication. In addition, oncogenic changes mismanage the regulation of reserve MCMs/CMGs, predicting tumor-selective deficiencies in CMG functionality and a tumor-specific CMG vulnerability relative to non-tumor cells (1,46). Coumermycin-A1, clorobiocin, and the methyl-substituted synthetic derivative referred to here as methylbiocin (MBC) are effective CMGi, competing with ATP for binding and hydrolysis at one or more ATPase sites within the MCM ring.

Consistent with the above predictions, tumor cell viability is selectively reduced when cultured in the presence of low micromolar (1-4 μM) concentrations of these novel CMGi, and, importantly, a therapeutic window exists for targeting the CMG helicase with future derivatives of these CMGi. In agreement with our findings, the National Cancer Institute has publicly available growth suppression data showing that many solid tumor cell lines (NCI-60 set) are sensitive to similar low micromolar concentrations of the CMGi as tested here (CA1, NSC107412; clorobiocin, NSC227186), but are largely insensitive to novobiocin (NSC2382; https://dtp.cancer.gov/dtpstandard/dwindex/index.jsp). Until now, a cellular target(s) required for proliferation and consistent with these differences at low micromolar concentrations was not clear. It had been suggested that HSP90 might be a target, but the high doses required to inhibit HSP90 (700-1000 μM) and its sensitivity to both CA1 and novobiocin suggest it is not the target(47,48). The relationship between the low micromolar CMGi doses that inhibit the CMG enzyme in biochemical assays, and low doses that target the CMG in cells and inhibit tumor cell viability/proliferation, strongly suggests that the CMG helicase (and replisome) is a major target of these CMGi compounds. Interestingly, at higher doses novobiocin can inhibit a different anti-cancer target, DNA Polymerase-theta, which also contains a helicase domain and is involved in DNA repair (49). The inability of novobiocin to inhibit the CMG demonstrates that these aminocoumarins display target specificity and distinct modes of action that could be used in specific anti-cancer regimens.

Aminocoumarins were originally marketed or investigated as anti-bacterial agents, but their use was supplanted by more effective quinolones such as ciprofloxacin (18). Prior to losing clinical favor as antibiotics, aminocoumarin drug development advanced in synthesizing and testing a derivative called BL-C43 in human trials for sepsis. BL-C43 had excellent pharmacokinetic and pharmacodynamic (PK/PD) characteristics (18,50). Unfortunately, subjects treated with very high doses of BL-C43 (750 mg/day for 10-14 days) displayed unwanted side effects for an antibiotic, presenting with gastrointestinal (GI) and liver (jaundice) adverse events(18,50). Intriguingly, BL-C43 contains the complete CMGi warhead defined here and is quite likely capable of targeting the CMG helicase based on our results. This would suggest that the high-dose side effects might have been due, at least in part, to targeting the CMG helicase in proliferating cells of the GI tract and liver. However, the BL-C43 trial illustrates the potential for the development of CMGi drugs with excellent PK/PD features that could be used at lower dosing in anti-cancer trials in the future. Given the many roles of MCM/CMG complexes in cell cycle regulation, DNA replication, and DNA repair, future CMGi with drug-like qualities and distinct mechanisms of action will provide a novel means for cancer intervention. The CMGi discovered here will help inform the development of such derivatives.

### Methods

### Cell Lines and Inhibitors

Human keratinocytes (HaCaT; RRID:CVCL_0038), 143B cells (osteosarcoma; RRID:CVCL_2270), and HEK-293T (RRID:CVCL_0063) cells were maintained in Dulbecco's Modified Eagle's Medium (DMEM) supplemented with 10% fetal bovine serum (FBS; Peak Serum, PS-FB-2). Human Psn1 (pancreatic ductal adenocarcinoma; RRID:CVCL_1644) and H460 (non-small cell lung carcinoma; RRID:CVCL_0459) cells were cultured in RPMI-1640 medium supplemented with 10% FBS. HaCaT cells were synchronized in Go using serum starvation for 48 hr and released into the cell cycle by addition of DMEM with 10% FBS (51). Novobiocin (cat #46531) and Courmermycin-A1 (cat #C9270) were obtained from Sigma. Etoposide (cat #S1225) and AT7519 (cat #S1524) were obtained from SelleckChem. All stock solutions of inhibitors were stored at −20° C. as 10 mM suspensions in DMSO.

### Cell Viability Assays

Cell viability determinations were performed using CellTiter-Glo Assays (Promega; cat #G7572). Cells were seeded in 96-well plates at a density of ~3×10³ cells/well and treated with drugs for 72 hr, after which the cells were processed for viability using CellTiter-Glo reagent according to the instructions of the manufacturer. Each drug concentration test was performed using four replicates, and results averaged and plotted on graphs, +/−1 s.d.

### Immunoblotting and Antibodies

Immunoblotting was performed using standard enhanced chemiluminescent (ECL) and polyacrylamide gel techniques. Lysates from equal cell numbers were separated into Triton X-100-soluble or -resistant (chromatin-bound) protein fractions as described (37,52), and compared to whole-cell protein lysates. All cell lysates were supplemented with protease inhibitors (1 mM PMSF, 1 mM benzamidine, 0.15 μM Aprotinin, 4 μM Leupeptin, 1 μM Antipain). Immunoblots were assessed with the following antibodies (all used at 1:500-1:1000 dilutions): from Santa Cruz: anti-Mcm5 (sc-165994, RRID:AB_2142526), anti-Mcm6 (sc-55577, RRID:AB_831540), anti-Mcm7 (sc-9966, RRID:AB_627235), anti-Orc2 (sc-32734, RRID:AB_2157726), anti-Cdt1 (sc-28262, RRID:AB_2076885), anti-Cdc6 (sc-9964, RRID:AB_627236), anti-phospho-Ser54-Cdc6 (sc-12920-R, RRID:AB_668066), anti-DNA polymerase e (sc-12728, RRID:AB_675496), anti-DNA polymerase 6 (sc-17776, RRID:AB_675487), anti-y-H2AX (sc-517348, RRID:AB_2783871), anti-Sld5 (sc-398784, RRID:AB_2940776); from Abcam: anti-Psf1 (ab183524, RRID:AB_2922402), and anti-Psf3 (ab254855, RRID:AB_2940777), anti-phospho-Ser53-Mcm2 (ab109133, RRID:AB_10863901), anti-DNA polymerase a. (ab31777, RRID:AB_731976); from Proteintech: anti-Psf2 (16247-1-AP, RRID:AB_2111895), anti-GAPDH (60004-1-lg, RRID:AB_2107436); from Cell Signaling: anti-RPA32 (2208s, RRID:AB_2238543), anti-RPA70 (2267s, RRID:AB_2180506), anti-Mcm3 (4003s, RRID:AB_2142261), anti-Lamin A/C (20325, RRID:AB_2136278), anti-WRN (4666, RRID:AB_10692114), anti-BLM (2742, RRID:AB_2064649), anti-phospho-Ser139-Mcm2 (12958, RRID:AB_2798069), anti-phospho-Ser345-Chk1 (2348, RRID:AB_331212), anti-cleaved Parp (5625, RRID:AB_10699459), anti-phospho-Thr320-PP1-ca (2581, RRID:AB_330823), anti-Rb (9313, RRID:AB_1904119), anti-phospho-Ser807/811-Rb (9308, RRID:AB_331472); from Sigma-Aldrich: anti-Flag (F3165, RRID:AB_259529), anti-beta-actin (A5441, RRID:AB_476744), rabbit IgG (15006, RRID:AB_1163659), anti-Flag agarose (A2220, RRID:AB_10063035), anti-Mouse IgG agarose (A6531, RRID:AB_258295); from Biolegend: anti-And-1 (Ctf4) (630301, RRID:AB_2215084); from Bethyl Laboratory: anti-Mcm10 (A300-131A, RRID:AB_2142119); from Invitrogen: anti-PPI-c (MA5-15589, RRID:AB_10980092); from BD Pharmingen: anti-Mcm4 (559544, RRID:AB_397267). Chicken polyclonal anti-Cdc45, rabbit polyclonal anti-Cdc6, and rabbit polyclonal anti-Mcm2 (used at 1:2000 dilutions) were generated by our group and validated as described (8).

### BrdU Labeling and Immunofluorescence Techniques

Verification of synchronization and determination of drug effects was performed by measuring the incorporation of bromo-deoxyuridine (BrdU) into replicating foci within nuclei. At times indicated, cells were pulse-labeled with 15 μM BrdU for 30 min, fixed with 4% paraformaldehyde, and analyzed by standard immunofluorescent techniques(40,51) with an anti-BrdU monoclonal antibody (Roche, clone no. BMC9318, RRID:AB_2313622). The average counts of three fields of 100 or more cells were used to determine the percentages of BrdU-labeled nuclei, +/−1 s.d.

### Topoisomerase II Decatenation Assay

The Topoisomerase II drug screening kit (kDNA-based; TopoGEN, TG1009-1A) was used to assess in vitro Topo II enzyme activity according to the manufacture protocol. Assays were performed in 20 mL reactions with 4 μL of 5× Assay Buffer (0.25 M Tris-HCl (pH 8), 10 mM ATP, 0.75 M NaCl, 50 mM MgCl2, 2.5 mM Dithiothreitol, 150 mg/ml BSA), 1 mL kDNA (0.2 mg), 1 mL DMSO solvent or compounds at indicated concentrations, 1 mL Topoisomerase II (2 Units), 13 mL water. Assays were incubated at 37° C. for 30 min and stopped by addition of 2 mL 10% SDS and Proteinase K (50 mg/ml). After incubating at 37° C. for 15 min, samples were mixed with 1/10 volume of loading buffer and resolved on a 1% agarose gel.

### Co-Immunoprecipitation Assays

Nuclear extracts were prepared from synchronized HaCaT cells after releasing into S-phase (at 20 hrs post release) or HEK-293T cells expressing stable Flag-Mcm2 protein (human). Cells from one 10 cm dish were resuspended in 300 mL of Buffer A (10 mM Hepes-KOH pH 7.5, 10 mM KCl, 1.5 mM MgCl2, 0.34 M sucrose, 10% glycerol, protease inhibitors, and 0.1% Triton-X-100) and incubated on ice for 5 min. The nuclear pellet was obtained by centrifugation at 4,000 rpm at 4° C. for 5 min and washed with the Buffer A without Triton X-100. The nuclear extract was obtained by resuspending the nuclear pellet in 300 ml of Buffer A containing 420 mM potassium acetate and 0.01% Triton X-100, and incubating at 4° C. for 1 hr. The final concentration of potassium acetate in nuclear extracts was adjusted to 200 mM for the co-immunoprecipitation assays. For the co-immunoprecipitation assays, 10 mg of indicated antibody [anti-Mcm2, anti-Psf1, or rabbit IgG (Sigma)], or 30 mL of mouse IgG or anti-Flag agarose beads (Sigma), was incubated with 300 mL of nuclear extract at 4° C. for 4 hrs. Protein A/G agarose beads (30 mL, Santa Cruz, sc-2003) were added and incubated for 1 hr with antibodies. Agarose beads were washed once in Buffer A containing 200 mM potassium acetate and 0.01% Triton X-100 and incubated with 15 μM CA1 or DMSO (same concentration as in CA1 sample) for 30 min. Beads were washed in the same buffer and analyzed by immunoblotting with indicated antibodies.

### Purification of the Human CMG Helicase

The human CMG helicase was purified following the established and validated protocol of Hurwitz and colleagues(19,20), with minor modifications. A detailed description of the approach is shown here, to allow for comparisons to the methods described by the Hurwitz group.

High Five insect cells (2-3 million cells/ml, 1.5 L), were in cultured in a shaking incubator at 27° C. in suspension with ESF921 serum free medium (Expression Systems, CA; cat. #96-001-01), then co-infected for 60 hrs with 11 baculoviruses expressing human 6His2Flag-Cdc45, the human MCM hexamer (Mcm2, Mcm3, Mcm4, Mem5, Mcm6, Mcm7), and human GINS (GST-Sld5, Psf3, Psf2, and Psf1). Each virus was infected at an MOI of ~10 from individual virus stocks. Infected cells were harvested by centrifugation at 650×g for 5 min at 4° C., washed with cold PBS, frozen on dry ice, and stored at −80° C. until use. The cell pellet (~20 mL) was thawed on ice, resuspended in 45 ml Hypotonic Buffer [20 mM Hepes-NaOH (pH 7.5), 5 mM KCl, 1.5 mM MgCl2] with protease inhibitors (1 mM PMSF, 1 mM Benzamidine, 0.15 μM Aprotinin, 4 μM Leupeptin, and 1 μM Antipain), and kept on ice for 10 min before lysing by Dounce homogenization (tight fitting, 60 strokes). The cell extract was adjusted to 0.42 M potassium acetate and centrifuged at 43,000×g for 1 hr at 4° C. The cleared lysate was mixed with 0.75 ml glutathione beads (cat. #17-0756-05; GE Healthcare) pre-equilibrated with FEQ buffer [20 mM Hepes-NaOH (pH 7.5), 0.42 M potassium acetate, 5 mM KCl, 1.5 mM MgCl₂] and incubated by rotation at 4° C. overnight. Following centrifugation at 290×g at 4° C., the bound glutathione beads were washed four times (15 min each wash) with 40 ml of FW buffer [20 mM Hepes-NaOH (pH 7.5), 0.42 M potassium acetate, 1 mM DTT, 1 mM EDTA, 0.01% NP40, 10% glycerol (vol/vol) with protease inhibitors as above] containing protease inhibitors (as above). Bound proteins were eluted at 4° C. three times (1 hr each elution) with 3.5 ml Q buffer [20 mM Hepes-NaOH (pH 7.5), 1 mM DTT, 1 mM EDTA, 0.01% NP40, 10% glycerol (vol/vol), and protease inhibitors as above] containing 0.15 M potassium acetate and 20 mM reduced glutathione. The eluted fractions were combined and applied to a HiTrap Q-Sepharose FF column (HiTrap Q FF 1 mL, cat. #17505301, Cytiva), pre-equilibrated 3× with 5 ml of Q buffer containing 0.15 M, 0.75 M, 0.15M potassium acetate, in sequence. The Q-Sepharose FF column was washed with 10 ml of FW buffer containing protease inhibitors (as above). The proteins were eluted with 10 ml of Q buffer containing 0.75 M potassium acetate. The eluted fraction was mixed with 0.15 ml of anti-Flag M2 affinity gel/beads and incubated while rotating at 4° C. overnight. After centrifugation at 290×g for 5 min at 4° C., the beads were washed three times with 10 ml PreScission buffer [50 mM Tris-HCl (pH 7.5), 0.15 M NaCl, 1 mM DTT, 1 mM EDTA] for 15 min at 4° C., and eluted at 4° C. three times (1 hr each elution) with 0.2 ml PreScission enzyme buffer containing 0.2 mg/ml 3×-Flag peptides. The combined eluates were incubated with 20 Units of PreScission Protease (GE Healthcare) and 0.1 mL glutathione beads for 4 hr at 4° C. The supernatant was layered onto a 15-40% glycerol gradient [25 mM Tris-HCl (pH 7.5), 50 mM NaCl, 1 mM DTT, 1 mM EDTA, 0.01% NP40, protease inhibitors as above] in a 5 mL ultra-centrifugation tube and centrifuged at 260,000×g for 14 hr at 4° C. Glycerol fractions (0.15 mL each fraction) were collected from bottom of tube and stored at −80° C. until use. Typically, fractions 6-9 contained complete hCMG (~750-800 kDa) and co-migrated with thyroglobulin.

Estimation of hCMG protein amount isolated was determined by comparing to BSA standards. We nominally achieved purification of 5-6 ng/μL hCMG enzyme from three glycerol fractions, or ~7.5 fmol hCMG/μL. Across multiple preps this varied from 5-15 fmol hCMG/μL, consistent with that reported by Hurwitz and colleagues (19,20). The specific ATPase activity of our isolated hCMG enzyme was consistent with the hCMG ATPase activity obtained by Hurwitz and colleagues (19,20). This prior hCMG analysis determined that the human helicase hydrolyzes ATP to ADP at a rate of −80 mol-ADP per minute per mol-hCMG in the presence of 500 μM ATP. Using the ADP-sensing fluorescent-polarization assay (described below), 15 fmol (in 2 μL) of hCMG elicits an ~52% mP change relative to the assay window (see Results), equating to production of ~7-8 μM ADP in 1 hr (80×15 fmol×60 min=72 pmol ADP in a 10 μL reaction, or 7.2 μM ADP). This activity represents an ~1.5% ADP conversion rate by the hCMG and falls within the reliability range of Z′=0.6-0.8 for screening in the primary ATPase assay.

### Expression and Purification of SV40 TAg and E1 Helicases

The SV40 TAg was expressed in Sf9 cells by infecting the cells with 6His-2Flag-TAg baculovirus. The Sf9 cells (300 ml) were grown at 27° C. in suspension with ESF921 serum free medium (Expression System, 96-001-01) and infected with 6His-2Flag-TAg baculovirus at a density of 2-3 million cells/ml. After 70 hours, the cells were harvested by centrifugation at 500×g for 10 min at 4° C., washed with 30 ml cold PBS, and then frozen on dry ice and store at −80° C. until use. The frozen cell pellet was thawed on ice and resuspended with 8 ml hypotonic buffer (20 mM Hepes-NaOH (pH 7.5), 5 mM KCl, 1.5 mM MgCl₂) containing protease inhibitors (1 mM PMSF, 1 mM Benzamidine, 0.15 μM Aprotinin, 4 M Leupeptin, and 1 μM Antipain). The cell suspension was lysed by Dounce homogenization for 60 strokes and the lysate was adjusted to 0.42 M Potassium acetate followed by centrifugation at 18,800 rpm (SW55Ti) for 60 min at 4° C. The cleared lysate was mixed with 0.4 ml of anti-FLAG M2 affinity gel preequilibrated with FEQ buffer (20 mM Hepes-NaOH (pH 7.5), 0.42 M potassium acetate, 5 mM KCl, 1.5 mM MgCl2), and incubated and rotated overnight at 4° C. With centrifugation at 290×g for 5 min at 4° C., the beads were washed three times with 10 ml FW buffer (20 mM Hepes-NaOH (pH 7.5), 0.42 M Potassium acetate, 1 mM DTT, 1 mM EDTA, 0.01% NP40, 10% glycerol (vol/vol) with protease inhibitors). The bound TAg was eluted three times with 2 ml Q buffer (20 mM Hepes-NaOH (pH 7.5), 1 mM DTT, 1 mM EDTA, 0.01% NP40, 10% glycerol (vol/vol) with protease inhibitors) containing 0.15 M potassium acetate and 0.1 mg/ml 3× FLAG peptides (F4799, Sigma). The eluted proteins were combined and applied to a Q Sepharose Fast Flow column (HiTrap Q FF 1 ml, Cat #0.17505301, Cytiva) preequilibrated with Q buffer containing 0.15 M potassium acetate. The Q column was washed with 5 ml Q buffer containing 0.3 M Potassium acetate and eluted with 5 ml Q buffer containing 1 M Potassium acetate. The elution was further applied to an Ultra-15 centrifugal unit (UFC905096, 50K, Millipore) and centrifuged at 3,800×g at 4° C. until the volume is 250 μl. After adding 15 ml Storage buffer (20 mM Tris-HCl (pH 8.0), 200 mM NaCl, 2 mM DTT, 2 mM EDTA), the centrifugal unit was centrifuged again until the final volume is 250 μl. 250 μl of 100% glycerol was added to the final TAg protein solution and stored at −80° C.

The HPV16 or HPV18 E1 helicase domain (16E1HD or 18E1HD) was expressed and purified from Sf9 cells by infecting the cells with 2Flag-GST-E1 baculovirus. The cleared cell lysates were prepared as for large T antigen and incubated with Glutathione beads at 4° C. overnight. The bound E1 proteins were eluted from beads with 20 mM reduced glutathione in Q buffer, applied to a Q Sepharose Fast Flow column, and eluted with 0.75 M potassium acetate in Q buffer. Purified E1 proteins were obtained by further incubation with Flag-M2 beads and elution with Flag peptides.

### Helicase Assays

Helicase fork-unwinding assays were performed at 37° C. for 1 hr in 20 μL reaction volumes using 2-4 μl of hCMG (~15-30 fmol), in a buffer consisting of 25 mM Hepes-NaOH (pH 7.5), 5 mM NaCl, 0.5 mM ATP, 10 mM magnesium acetate, 1 mM DTT, 0.1 mg/ml bovine serum albumin (BSA), and ~10 fmol of radiolabeled DNA forks (20). Reactions were terminated with 4 μl of 6× stop solution [50 mM EDTA (pH 8.0), 40% (vol/vol) glycerol, 2% (wt/vol) SDS, and 0.3% bromophenol blue], loaded onto 10% (wt/vol) polyacrylamide gels, resolved at 150 volts in 1×-TBE buffer (89 mM Tris base, 89 mM boric acid, and 2 mM EDTA), dried on filter paper, and analyzed by autoradiography and quantified by PhosphorImager assessment. Assays using SV40 large T antigen helicase or HPV-E1 helicases were performed in the same conditions as those used for the hCMG helicase.

The DNA substrates were formed as described by Hurwitz and colleagues (20) by annealing two oligonucleotides: 10 pmol of M13-39-5′dT40 and 10 pmol of anti-M13-39-3′dT40 in the presence of 0.1 M NaCl by heating for 5 min at 95° C., followed by slow cooling to room temperature. The oligonucleotide M13-39-5′dT40 was 5′ end-labelled with T4 Polynucleotide Kinase (cat #M0201S; New England Biolabs) and [γ-³²P]-ATP before annealing. The annealed DNA substrates were resolved in 10% polyacrylamide gels in 1×-TBE buffer at 150 volts for 30 min. Bands containing double-stranded DNA forks were cut from the gel and forks eluted after crushing the gel with 200 μL Tris-EDTA buffer containing 0.15 M NaCl for 3 hr at 37° C. The eluted fraction was collected by centrifugation at 13,000 rpm at 4° C. for 10 min and stored at 4° C. until use.

Fluorescent-Polarization Measurements with ADP² Transcreener Assay

The fluorescent-polarization (FP) ADP-sensing assays were performed using the Transcreener® ADP² FP assay kit (cat #3010-1K, Bellbrook Labs). For hCMG inhibitor screening, assays were performed at 37° C. for 1 hr in 10 μl reactions in a 384-well plate (cat. #4514, Corning) using 2 μL of hCMG (~15 fmol), 25 mM Hepes-NaOH (pH 7.5), 10 mM NaCl, 0.5 mM ATP, 10 mM magnesium acetate, 1 mM DTT, 0.1 mg/ml BSA, and DNA fork substrates. But assays without DNA substrates can also be performed, since the hCMG does not require DNA to be present to hydrolyze ATP (20). Selected inhibitors or samples from a chemical library were added into the reactions when conducting the screening or testing inhibitor effectiveness. The NCI Diversity Set VI chemical library was obtained from the National Cancer Institute (Bethesda, MD).

The window of sensitivity for the ADP-sensing assays is determined by setting up two 10 μL control samples without any added hCMG helicase: a Low-FP mixture (4 nM ADP Alexa Fluor-633 Tracer alone) and a High-FP mixture (4 nM ADP Alexa Fluor-633 Tracer plus patented anti-ADP² Antibody). The amount of ADP² Antibody used had to be adjusted to account for the use of 500 μM ATP in our hCMG assays, versus 10 μM ATP in enzyme reactions typically assessed with standard kits prepared by BellBrook Labs. The ADP² Antibody has a significantly higher affinity for ADP compared to ATP, but since it can bind to ATP to some extent, this must be offset by including more ADP² Antibody in our high-ATP assays. This was done according to the manufacturer by performing a titration with increasing ADP² Antibody, fixed 4 nM ADP Alexa Fluor-633 Tracer, and 500 μM ATP to determine the ~EC₈₀₋₈₅ for millipol (mP) changes, which determined the optimal ADP² Antibody concentration to use as 0.64 mg/ml (FIG. 9A). Samples are read with a Perkin Elmer Envision II plate reader with optimized Cy5 (far-red) FP-compatible mirror and cubes (cat #2100-8390, Perkin Elmer). The Low-FP sample is the least polarized and gives a low mP reading, while the High-FP sample is the most polarized and gives the highest mP reading. The difference between the Low-FP and High-FP values defines the FP assay window, which is normally in a range of 150-200 mP under ideal conditions for screening purposes. The hCMG helicase hydrolyzes ATP to ADP and decreases the mP reading within this window, with an ideal change (ΔmP) in the FP window of at least 50% to be in a readable range for inhibitor screening (as per manufacturer). Potential hCMG chemical inhibitors reverse this effect and cause the mP readings to increase.

We performed a titration with increasing and decreasing concentrations of ADP and ATP, respectively, to assess the sensitivity of the FP assay in detecting ADP production (FIG. 9B). Consistent with the stated manufacturer predictions, the assay can reliably detect 1-3% changes in ADP production (i.e., 5-15 μM ADP production) in starting concentrations of ATP of 500 μM, with a Z′ efficiency between 0.6-0.8 (determined following manufacturer protocols). We also note that ATP-gamma-S cannot be used as a positive control for inhibition of the CMG in this ADP-sensing assay, as it competes with the anti-ADP² Antibody and alters the detection window by itself (FIG. 9C).

Once the hCMG enzyme reactions are complete, 10 μL of 1× Stop Solution and Detection Buffer (buffer from BellBrook Labs; containing 4 nM ADP Alexa Fluor-633 Tracer plus ADP² Antibody) are added to each reaction, and to the High-FP control. The Low-FP control receives 10 μL of 1× Stop Solution and Detection Buffer containing only 4 nM ADP Alexa Fluor-633 Tracer (no Antibody). Samples are incubated at 25° C. for 1 hr, then read in the plate reader. Potential positive inhibitors (hits) of the hCMG are verified to be incapable of altering the mP window on their own, by artificially raising the mP readings to appear more polarized as occurs when the hCMG is truly inhibited by a compound. For this, potential hCMG inhibitors are added to a 10 μL mixture containing 25 mM Hepes-NaOH (pH 7.5), 10 mM NaCl, 0.5 mM ATP, 10 mM magnesium acetate, 1 mM DTT, 0.1 mg/ml BSA, and no hCMG, and mixed with 10 μL of Stop Solution and Detection Buffer (4 nM ADP Alexa Fluor®633 Tracer without ADP² Antibody). Plate readings from these tests of potential positive hits should be similar to that seen with the Low-FP control, or such hits may instead be false-positives.

### In Silico Docking Parameters

The structure of the human CMG helicase determined at 3.3 Angstroms by electron cryomicroscopy (cryo-EM) with bound ADP, ATP-gamma-S, or no nucleotide within individual ATP clefts, and with bound DNA (PDB accession code 6XTX) was used for in silico docking with Autodock Vina Version 1.2.3 (RRID:SCR_011958) (33). Estimated interaction/binding energies were calculated by Autodock. The conversion of protein and small chemical molecules into PDBQT format and grid box generation were achieved by using Autodock Tools Version 1.5.6. Each pair of MCMs forming an ATP cleft was extracted and the grid box derived with the active site in the center of the cleft. The docking configurations of clorobiocin were viewed and analyzed using Pymol Version 2.4.1 (RRID:SCR_000305).

### Chemical Synthesis Methods

### REFERENCES FOR CHEMICAL SYNTHESIS METHODS

(1) Gunaherath, G. M. K. B.; Marron, M. T.; Wijeratne, E. M. K.; Whitesell, L.; Gunatilaka, A. A. L. Synthesis and biological evaluation of novobiocin analogues as potential heat shock protein 90 inhibitors. Bioorg. Med. Chem. 2013, 21 (17), 5118-5129. DOI: 10.1016/j.bmc.2013.06.042.
(2) Mandler, M. D.; Baidin, V.; Lee, J.; Pahil, K. S.; Owens, T. W.; Kahne, D. Novobiocin Enhances Polymyxin Activity by Stimulating Lipopolysaccharide Transport. J. Am. Chem. Soc. 2018, 140 (22), 6749-6753. DOI: 10.1021/jacs.8b02283.
(3) Olson, S. H.; Slossberg, L. H. Synthesis of coumermycin A1. Tetrahedron Lett. 2003, 44 (1), 61-63. DOI: 10.1016/s0040-4039(02)02487-5.

General Chemistry Information. All reagents were purchased from commercial suppliers and used without further purification. All ¹H NMR spectra were recorded on a Bruker Ascend 500 MHz spectrometer, equipped with cryoprobe, using CDCl₃, CD₃OD or DMSO-d₆ as the solvent. All ¹³C NMR spectra are recorded at 125 MHz. Coupling constants are measured in Hertz (Hz) and the chemical shifts (δ_{H} and δ_{C}) are quoted in parts per million (ppm) relative to TMS (δ 0), which was used as the internal standard. High resolution mass spectroscopy was carried out on an Agilent 6210 LC/MS (ESI-TOF) instrument. Microwave reactions were performed in CEM or Biotage initiator 8 machines. HPLC analysis was performed using a JASCO HPLC system equipped with a PU-2089 Plus quaternary gradient pump and a UV-2075 Plus UV-VIS detector, using an Alltech Kromasil C-18 column (150×4.6 mm, 5 μm) and Agilent Eclipse XDB-C18 (150×4.6 mm, 5 μm). High Resolution Mass Spectroscopy (HRMS) was recorded on Agilent 6230LC-MS TOF mass spectrometer. Thin layer chromatography was performed using silica gel 60 F254 plates (Fisher), with observation under UV when necessary. Anhydrous solvents (acetonitrile, dimethylformamide, ethanol, isopropanol, methanol and tetrahydrofuran) were used as purchased from Aldrich. Burdick and Jackson HPLC grade solvents (methanol, acetonitrile and water) were purchased from VWR for HPLC and high resolution mass analysis. HPLC grade TFA was purchased from Fisher.

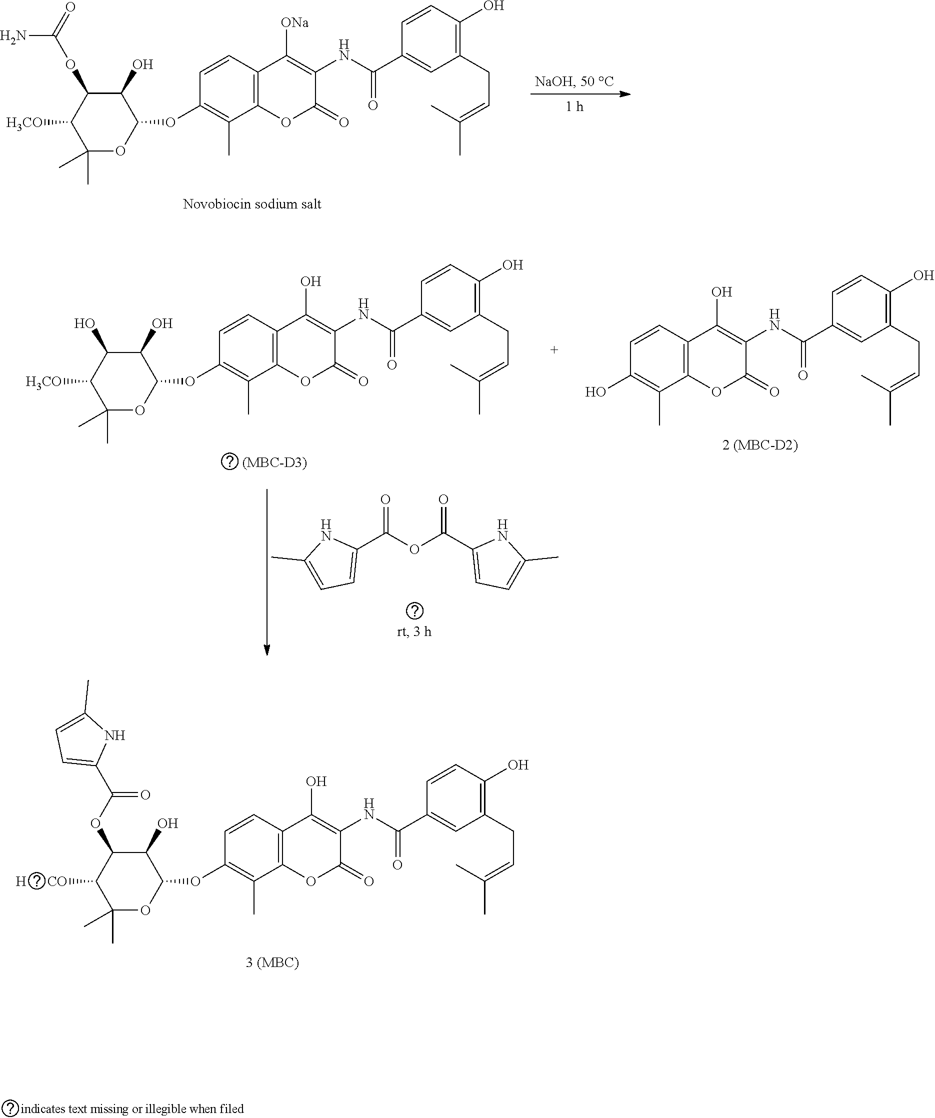

### N-(7-(((2R,3R,4S,5R)-3,4-Dihydroxy-5-methoxy-6,6-dimethyltetrahydro-2H-pyran-2-yl)oxy)-4-hydroxy-8-methyl-2-oxo-2H-chromen-3-yl)-4-hydroxy-3-(3-methylbut-2-en-1-yl)benzamide (1, MBC-D3).^{1, 2}

Commercially available Novobiocin sodium salt (5.00 g, 7.88 mmol) was dissolved in aqueous NaOH (50 mL, 1 M) and heated to 50° C. in a capped round bottom flask. After 1 h, the reaction mixture was cooled to 0° C. and neutralized with 1 M HCl. The precipitate that formed was collected and the aqueous layer was extracted with DCM (50 mL×5). The organic layer was dried (Na₂SO₄), filtered, concentrated, combined with the precipitate, and purified via SiO₂ chromatography using a Biotage Isolera system with 25%-50% EtOAc in DCM to afford 1 (MBC-D3) as a pale yellow solid (3.05 g, 68%) and 2 (MBC-D2) as a pale yellow solid (0.137 g, 3%). ¹H NMR (500 MHz, DMSO-d₆) δ 11.95 (s, 1H), 10.04 (s, 1H), 9.21 (s, 1H), 7.89-7.57 (m, 3H), 7.17 (d, J=9.0 Hz, 1H), 6.86 (d, J=8.4 Hz, 1H), 5.52 (d, J=2.2 Hz, 1H), 5.34-5.28 (m, 2H), 5.01 (d, J=6.1 Hz, 1H), 4.03-3.97 (m, 1H), 3.91 (br s, 1H), 3.49 (s, 3H), 3.29-3.25 (m, 3H), 2.20 (s, 3H), 1.70 (s, 6H), 1.25 (s, 3H), 1.02 (s, 3H). HRMS (ESI⁺): m/z calculated for C₃₀H₃₆NO₁₀ (M+H)⁺ 570.2339, found 570.2329; HPLC-MS (ESI⁺): m/z 569.6 (M+H)⁺. N-(4,7-Dihydroxy-8-methyl2-oxo-2H-chromen-3-yl)-4-hydroxy-3-(3-methylbut-2-en-1-yl)benzamide (2, MBC-D2).^{2 1}H NMR (500 MHz, DMSO-d₆) δ 11.81 (s, 1H), 10.41 (s, 1H), 10.04 (s, 1H), 9.17 (s, 1H), 7.80-7.66 (m, 2H), 7.57 (d, J=8.7 Hz, 1H), 6.88 (d, J=8.7 Hz, 1H), 6.86 (d, J=8.4 Hz, 1H), 5.31 (t, J=7.2 Hz, 1H), 3.27 (d, J=7.2 Hz, 2H), 2.17 (s, 3H), 1.70 (s, 6H); HRMS (ESI+): m/z calculated for C₂₂H₂₂NO₆ (M+H)⁺ 396.1447, found 396.1443; HPLC-MS (ESI+): m/z 395.9 (M+H)⁺, 812.8 (2M+Na)⁺.

(3R,4S,5R,6R)-5-Hydroxy-6-((4-hydroxy-3-(4-hydroxy-3-(3-methylbut-2-en-1-yl)benzamido)-8-methyl-2-oxo-2H-chromen-7-yl)oxy)-3-methoxy-2,2-dimethyltetrahydro-2H-pyran-4-yl (3, MBC).^{1, 2} Compound 1 (0.100 g, 0.176 mmol) was dissolved in anhydrous MeCN (5 mL) under an atmosphere of argon. 5-Methyl-1Hpyrrole-2-carboxylic anhydride³ (0.080 g, 0.34 mmol) was added and stirred at room temperature for 5 min. While stirring, Sc(OTf)₃ [(85 μL of a stock solution prepared from 100 mg in anhydrous acetonitrile (1 mL)] was prepared and added to the reaction mixture. After 3 h, the reaction mixture was concentrated under reduced pressure and purified via SiO₂ chromatography using a Biotage Isolera system with 20%-25% EtOAc in DCM to afford 3 (MBC) as a white solid (0.022 g, 19%). ¹H NMR (500 MHz, DMSO-d₆) δ 11.98 (s, 1H), 11.65 (s, 1H), 10.04 (s, 1H), 9.22 (s, 1H), 7.76-7.70 (m, 3H), 7.20 (d, J=9.0 Hz, 1H), 6.86 (d, J=8.4 Hz, 1H), 6.78 (app t, J=2.9 Hz, 1H), 5.93 (app t, J=2.6 Hz, 1H), 5.67 (d, J=5.0 Hz, 1H), 5.62 (d, J=2.5 Hz, 1H), 5.49 (dd, J=9.8, 2.9 Hz, 1H), 5.31 (t, J=7.2 Hz, 1H), 4.19-4.15 (m, 1H), 3.66 (d, J=9.8 Hz, 1H), 3.47 (s, 3H), 3.27 (d, J=7.3 Hz, 2H), 2.26 (s, 3H), 2.25 (s, 3H), 1.70 (s, 6H), 1.30 (s, 3H), 1.10 (s, 3H). HRMS (ESI+) C₃₆H₄₀N₂O₁₁Na (M+Na)⁺ 699.2530, found 699.2519; HPLC-MS (ESI+): m/z 676.9 (M+H)⁺, 698.9 (M+Na)⁺

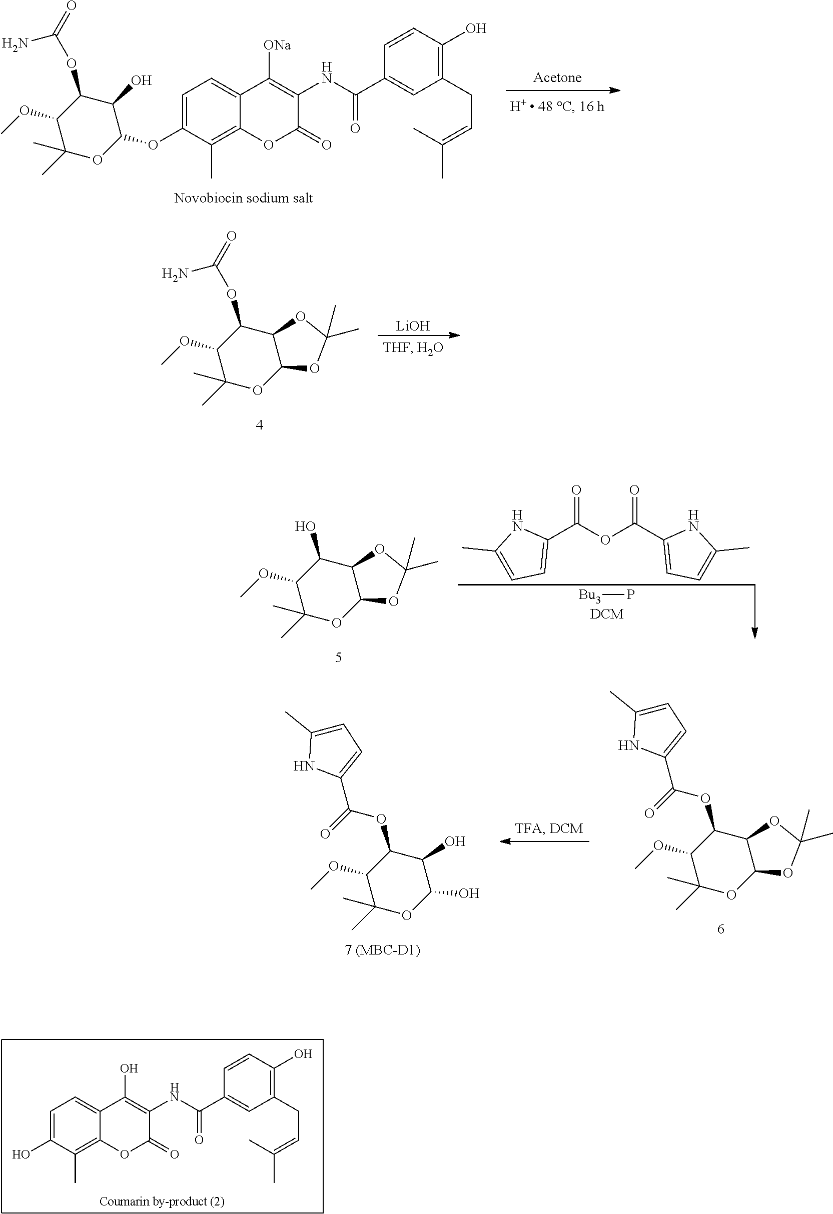

(3aR,6R,7R,7aR)-6-Methoxy-2,2,5,5-tetramethyltetrahydro-5H-[1,3]dioxolo[4,5-b]pyran-7-yl carbamate (4).³ A stirring mixture of Novobiocin sodium salt (1.00 g, 1.58 mmol) and para-toluene sulfonic acid (543 mg, 3.15 mmol) was heated at 48° C. for 16 hours. The reaction was monitored by HPLC-MS, and LCMS indicated consumption of the starting material, formation of coumarin by-product and formation of noviose intermediate 4. The crude mixture was purified by Al2O3 chromatography using ethyl acetate as the eluent to give UV active coumarin by-product and non UV active noviose carbamate 4 (96.9 mg, 0.352 mmol, 32%) as a white solid. This reaction was scaled-up with Novobiocin (8.0 g) to obtain 761 mg (38% yield) of the noviose intermediate 4. ¹H NMR (500 MHz, DMSO-d₆) d 6.86 (br s, 1H), 6.54 (br s, 1H), 5.31 (d, J=2.5 Hz, 1H), 4.95 (dd, J=10.0, 4.0 Hz, 1H), 4.21 (dd, J=4.0, 2.0 Hz, 1H), 3.43 (s, 3H), 3.22 (d, J=10.0 Hz, 1H), 1.44, 1.26, 1.19, 1.07 (4×s, each 3H); HPLC-MS (ESI+): m/z 298 (M+Na)⁺, 573 (2M+Na)⁺.

(3aR,6R,7R,7aR)-6-Methoxy-2,2,5,5-tetramethyltetrahydro-5H-[1,3]dioxolo[4,5-b]pyran-7-ol (5).3 The intermediate sugar-acetonide 4 (601.2 mg, 2.18 mmol) was dissolved in methanol (6.0 mL) and THF (6.0 mL). LiOH·H₂O (696.8 mg, 16.60 mmol) was dissolved in water (6.0 mL) and added to the stirring solution of sugar acetonide 4. The reaction mixture was warmed to 40° C. and stirred for 16 h. The reaction mixture was poured into water (30 mL) and extracted with EtOAc (3×30 mL). The combined organic phase was dried (MgSO₄), filtered and concentrated to obtain compound 5 (359.6 mg, 71%) as a white solid. ¹H NMR (500 MHz, DMSO-d₆) d 5.19 (d, J=2.2 Hz, 1H), 5.11 (d, J=6.7 Hz, 1H), 4.07 (dd, J=4.0, 2.2 Hz, 1H), 3.77 (ddd, J=10.3, 6.7, 3.9 Hz, 1H), 3.47 (s, 3H), 3.05 (d, J=9.8 Hz, 1H), 1.44, 1.27, 1.16, 1.01 (4×s, each 3H); HPLC-MS (ESI+): m/z 255 (M+Na)⁺, 487 (2M+Na)⁺.

(3aR,6R,7R,7aR)-6-methoxy-2,2,5,5-tetramethyltetrahydro-5H-[1,3]dioxolo[4,5-b]pyran-7-yl 5-methyl-1H-pyrrole-2-carboxylate (6).³ This was prepared by the method of Olson and Slossberg.³ The noviose sugar 5 (200 mg, 0.861 mmol) and 5-methyl-1H-pyrrole-2-carboxylic anhydride (from³ ethyl 5-methylpyrrole-2-carboxylate) (389.9 mg, 1.68 mmol) were dissolved in dry dichloromethane (12 mL) in a 25 mL round bottom flask under argon. Tributylphosphine (232 μL, 0.93 mmol) was added and the mixture stirred at room temperature for 60 h. The solution was added to water (10 mL) and extracted with dichloromethane (2×20 mL). The organic layers were combined, dried (MgSO₄), filtered, and concentrated under vacuum. The residue was purified by SiO₂ chromatography (10-80% EtOAc in hexanes) to provide compound 6 (208 mg, 71%). ¹H NMR (500 MHz, DMSO-d₆) δ 11.69 (s, 1H), 6.76 (app t, J=3.0 Hz, 1H), 5.92 (app t, J=3.0 Hz, 1H), 5.37 (d, J=2.1 Hz, 1H), 5.21 (dd, J=10.1, 4.0 Hz, 1H), 3.44 (s, 3H), 3.42 (d, J=10.1 Hz, 1H), 2.23 (s, 3H), 1.48 (s, 3H), 1.25 (s, 3H), 1.23 (s, 3H), 1.13 (s, 3H); HPLC-MS (ESI+): m/z 282.0 (M+H)⁺, 701.0 (2M+Na)⁺.

(3R,4S,5R)-5,6-Dihydroxy-3-methoxy-2,2-dimethyltetrahydro-2H-pyran-4-y 5-methyl-1H-pyrrole-2-carboxylate (7, MBC_D1).³ Compound 6 (0.05 g, 0.187 mmol) was dissolved in TFA/H₂O/DCM (62 μL:248 μL:2.75 mL) (1:4:45) and stirred at room temperature. After 30 min., TFA (62 L) was added and more TFA (62 L) was added after 4 h. After TLC indicated completion of the reaction, solid NaHCO₃ was added until a pH of 8 was reached. Na₂SO₄ was added to remove H₂O, and the solid was filtered off. The filtrate was concentrated under vacuum. The crude product was purified via SiO₂ chromatography using a gradient of 25%-75% EtOAc in hexanes to afford 7 (MBC-D1) as a mixture of α/β anomers (28.6 mg, 65%) as a white solid. ¹H NMR of the major anomer: ¹H NMR (500 MHz, CDCl₃) δ 8.98 (s, 1H), 6.90-6.86 (m, 1H), 6.01-5.96 (m, 1H), 5.21-5.16 (m, 1H), 5.02 (s, 1H), 4.15-4.10 (m, 1H), 3.53-3.44 (m, 4H), 2.33 (s, 3H), 1.38 (s, 3H), 1.26 (s, 3H); HRMS (ESI+) calculated for C₁₄H₂₁NO₆Na [M+Na]⁺ 322.1267, found 322.1265; HPLC-MS (ESI+) m/z 300.2 (M+H)⁺, 621.3 (2M+Na)⁺.

### Quantification and Statistical Analysis

All the statistical details of experiments can be found in the figure legends or Methods descriptions. Statistical analyses were performed with Prism (GraphPad). Error bars represent mean±SD. Statistical comparisons were analyzed with unpaired two-tailed Student's t-test. Data were considered statistically different at P<0.05.

### REFERENCES CITED IN EXAMPLE 2

1. Xiang S, Reed D R, Alexandrow M G. The CMG helicase and cancer: a tumor “engine” and weakness with missing mutations. Oncogene 2023; 42(7):473-90 doi 10.1038/s41388-022-02572-8.
2. Costa A, Diffley J F X. The Initiation of Eukaryotic DNA Replication. Annu Rev Biochem 2022; 91:107-31 doi 10.1146/annurev-biochem-072321-110228.
3. Gambus A, Jones R C, Sanchez-Diaz A, Kanemaki M, van Deursen F, Edmondson R D, et al. GINS maintains association of Cdc45 with MCM in replisome progression complexes at eukaryotic DNA replication forks. Nature cell biology 2006; 8(4):358-66 doi 10.1038/ncb1382.
4. Ilves I, Petojevic T, Pesavento J J, Botchan M R. Activation of the MCM2-7 helicase by association with Cdc45 and GINS proteins. Mol Cell 2010; 37(2):247-58 doi S1097-2765(09)00963-0 [pii]10.1016/j.molcel.2009.12.030.
5. Remus D, Beuron F, Tolun G, Griffith J D, Morris E P, Diffley J F. Concerted loading of Mcm2-7 double hexamers around DNA during DNA replication origin licensing. Cell 2009; 139(4):719-30 doi 10.1016/j.cell.2009.10.015.
6. Tada S, Blow J J. The replication licensing system. Biol Chem 1998; 379(8-9):941-9.
7. Walter J, Newport J. Initiation of eukaryotic DNA replication: origin unwinding and sequential chromatin association of Cdc45, RPA, and DNA polymerase alpha. Mol Cell 2000; 5(4):617-27 doi 10.1016/s1097-2765(00)80241-5.
8. Wong P G, Winter S L, Zaika E, Cao T V, Oguz U, Koomen J M, et al. Cdc45 limits replicon usage from a low density of preRCs in mammalian cells. PLoS ONE 2011; 6(3):e17533 doi 10.1371/journal.pone.0017533.
9. Ge X Q, Jackson D A, Blow J J. Dormant origins licensed by excess Mcm2-7 are required for human cells to survive replicative stress. Genes Dev 2007; 21:3331-41.
10. Woodward A M, Gohler T, Luciani M G, Oehlmann M, Ge X, Gartner A, et al. Excess Mcm2-7 license dormant origins of replication that can be used under conditions of replicative stress. J Cell Biol 2006; 173(5):673-83.
11. Ibarra A, Schwob E, Mendez J. Excess MCM proteins protect human cells from replicative stress by licensing backup origins of replication. Proc Natl Acad Sci USA 2008; 105(26):8956-61.
12. Bryant V L, Elias R M, McCarthy S M, Yeatman T J, Alexandrow M G. Suppression of Reserve MCM Complexes Chemosensitizes to Gemcitabine and 5-Fluorouracil. Mol Cancer Res 2015 doi 10.1158/1541-7786.MCR-14-0464.
13. Sedlackova H, Rask M B, Gupta R, Choudhary C, Somyajit K, Lukas J. Equilibrium between nascent and parental MCM proteins protects replicating genomes. Nature 2020; 587(7833):297-302 doi 10.1038/s41586-020-2842-3.
14. Ekholm-Reed S, Mendez J, Tedesco D, Zetterberg A, Stillman B, Reed S I. Deregulation of cyclin E in human cells interferes with prereplication complex assembly. The Journal of cell biology 2004; 165(6):789-800 doi 10.1083/jcb.200404092.
15. Nepon-Sixt B S, Bryant V L, Alexandrow M G. Myc-driven chromatin accessibility regulates Cdc45 assembly into CMG helicases. Commun Biol 2019; 2:110 doi 10.1038/s42003-019-0353-2.
16. Srinivasan S V, Dominguez-Sola D, Wang L C, Hyrien O, Gautier J. Cdc45 is a critical effector of myc-dependent DNA replication stress. Cell Rep 2013; 3(5):1629-39 doi 10.1016/j.celrep.2013.04.002.
17. Dominguez-Sola D, Ying C Y, Grandori C, Ruggiero L, Chen B, Li M, et al. Non-transcriptional control of DNA replication by c-Myc. Nature 2007; 448(7152):445-51.
18. Bisacchi G S, Manchester J I. A New-Class Antibacterial-Almost. Lessons in Drug Discovery and Development: A Critical Analysis of More than 50 Years of Effort toward ATPase Inhibitors of DNA Gyrase and Topoisomerase I V. ACS Infect Dis 2015; 1(1):4-41 doi 10.1021/id500013t.
19. Kang Y H, Farina A, Bermudez V P, Tappin I, Du F, Galal W C, et al. Interaction between human Ctf4 and the Cdc45/Mcm2-7/GINS (CMG) replicative helicase. Proceedings of the National Academy of Sciences of the United States of America 2013; 110(49):19760-5 doi 10.1073/pnas.1320202110.
20. Kang Y H, Galal W C, Farina A, Tappin I, Hurwitz J. Properties of the human Cdc45/Mcm2-7/GINS helicase complex and its action with DNA polymerase epsilon in rolling circle DNA synthesis. Proceedings of the National Academy of Sciences of the United States of America 2012; 109(16):6042-7 doi 10.1073/pnas.1203734109.
21. Peterson E J, Janzen W P, Kireev D, Singleton S F. High-throughput screening for RecA inhibitors using a transcreener adenosine 5′-O-diphosphate assay. Assay Drug Dev Technol 2012; 10(3):260-8 doi 10.1089/adt.2011.0409.
22. Yin T, Lallena M J, Kreklau E L, Fales K R, Carballares S, Torrres R, et al. A novel CDK9 inhibitor shows potent antitumor efficacy in preclinical hematologic tumor models. Mol Cancer Ther 2014; 13(6):1442-56 doi 10.1158/1535-7163.MCT-13-0849.
23. Yao S, Nguyen T V, Rolfe A, Agrawal A A, Ke J, Peng S, et al. Small Molecule Inhibition of CPS1 Activity through an Allosteric Pocket. Cell Chem Biol 2020; 27(3):259-68 e5 doi 10.1016/j.chembiol.2020.01.009.
24. Ho H, Miu A, Alexander M K, Garcia N K, Oh A, Zilberleyb I, et al. Structural basis for dual-mode inhibition of the ABC transporter MsbA. Nature 2018; 557(7704):196-201 doi 10.1038/s41586-018-0083-5.
25. Simon N E, Schwacha A. The Mcm2-7 replicative helicase: a promising chemotherapeutic target. BioMed research international 2014; 2014:549719 doi 10.1155/2014/549719.
26. Simon N, Bochman M L, Seguin S, Brodsky J L, Seibel W L, Schwacha A. Ciprofloxacin is an inhibitor of the Mcm2-7 replicative helicase. Biosci Rep 2013; 33(5) doi 10.1042/BSR20130083.
27. Vanden Broeck A, McEwen A G, Chebaro Y, Potier N, Lamour V. Structural Basis for DNA Gyrase Interaction with Coumermycin A1. J Med Chem 2019; 62(8):4225-31 doi 10.1021/acs.jmedchem.8b01928.
28. Flatman R H, Eustaquio A, Li S M, Heide L, Maxwell A. Structure-activity relationships of aminocoumarin-type gyrase and topoisomerase I V inhibitors obtained by combinatorial biosynthesis. Antimicrob Agents Chemother 2006; 50(4):1136-42 doi 10.1128/AAC.50.4.1136-1142.2006.
29. Hooper D C, Wolfson J S, McHugh G L, Winters M B, Swartz M N. Effects of novobiocin, coumermycin A1, clorobiocin, and their analogs on Escherichia coli DNA gyrase and bacterial growth. Antimicrob Agents Chemother 1982; 22(4):662-71 doi 10.1128/AAC.22.4.662.
30. Bochman M L, Bell S P, Schwacha A. Subunit organization of Mcm2-7 and the unequal role of active sites in ATP hydrolysis and viability. Mol Cell Biol 2008.
31. Coster G, Frigola J, Beuron F, Morris E P, Diffley J F. Origin licensing requires ATP binding and hydrolysis by the MCM replicative helicase. Mol Cell 2014; 55(5):666-77 doi 10.1016/j.molcel.2014.06.034.
32. Kang S, Warner M D, Bell S P. Multiple functions for Mcm2-7 ATPase motifs during replication initiation. Mol Cell 2014; 55(5):655-65 doi 10.1016/j.molcel.2014.06.033.
33. Rzechorzek N J, Hardwick S W, Jatikusumo V A, Chirgadze D Y, Pellegrini L. CryoEM structures of human CMG-ATPgammaS-DNA and CMG-AND-1 complexes. Nucleic Acids Res 2020; 48(12):6980-95 doi 10.1093/nar/gkaa429.
34. Olson S H, Slossberg L H. Synthesis of coumermycin A. Tetrahedron Lett 2003; 44(1):61-3 doi Pii S0040-4039(02)02487-5 Doi 10.1016/S0040-4039(02)02487-5.
35. Gunaherath G M, Marron M T, Wijeratne E M, Whitesell L, Gunatilaka A A. Synthesis and biological evaluation of novobiocin analogues as potential heat shock protein 90 inhibitors. Bioorg Med Chem 2013; 21(17):5118-29 doi 10.1016/j.bmc.2013.06.042.
36. Mandler M D, Baidin V, Lee J, Pahil K S, Owens T W, Kahne D. Novobiocin Enhances Polymyxin Activity by Stimulating Lipopolysaccharide Transport. J Am Chem Soc 2018; 140(22):6749-53 doi 10.1021/jacs.8b02283.
37. Mukherjee P, Winter S L, Alexandrow M G. Cell cycle arrest by transforming growth factor beta1 near G/S is mediated by acute abrogation of prereplication complex activation involving an Rb-MCM interaction. Mol Cell Biol 2010; 30(3):845-56.
38. Douglas M E, Ali F A, Costa A, Diffley J F X. The mechanism of eukaryotic CMG helicase activation. Nature 2018; 555(7695):265-8 doi 10.1038/nature25787.
39. Tsuji T, Ficarro S B, Jiang W. Essential role of phosphorylation of MCM2 by Cdc7/Dbf4 in the initiation of DNA replication in mammalian cells. Mol Biol Cell 2006; 17(10):4459-72 doi 10.1091/mbc.e06-03-0241.
40. Alexandrow M G, Hamlin J L. Cdc6 chromatin affinity is unaffected by serine-54 phosphorylation, s-phase progression, and overexpression of cyclin a. Mol Cell Biol 2004; 24(4):1614-27.
41. Lundberg A S, Weinberg R A. Functional inactivation of the retinoblastoma protein requires sequential modification by at least two distinct cyclin-cdk complexes. Mol Cell Biol 1998; 18(2):753-61.
42. Kwon Y G, Lee S Y, Choi Y, Greengard P, Nairn A C. Cell cycle-dependent phosphorylation of mammalian protein phosphatase 1 by cdc2 kinase. Proc Natl Acad Sci USA 1997; 94(6):2168-73 doi 10.1073/pnas.94.6.2168.
43. Jones M L, Baris Y, Taylor M R G, Yeeles J T P. Structure of a human replisome shows the organisation and interactions of a DNA replication machine. EMBO J 2021; 40(23):e108819 doi 10.15252/embj.2021108819.
44. Kilkenny M L, Simon A C, Mainwaring J, Wirthensohn D, Holzer S, Pellegrini L. The human CTF4-orthologue AND-1 interacts with DNA polymerase alpha/primase via its unique C-terminal HMG box. Open Biol 2017; 7(11) doi 10.1098/rsob.170217.
45. Jenkyn-Bedford M, Jones M L, Baris Y, Labib K P M, Cannone G, Yeeles J T P, et al. A conserved mechanism for regulating replisome disassembly in eukaryotes. Nature 2021; 600(7890):743-7 doi 10.1038/s41586-021-04145-3.
46. Reed D R, Alexandrow M G. Myc and the Replicative CMG Helicase: The Creation and Destruction of Cancer: Myc Over-Activation of CMG Helicases Drives Tumorigenesis and Creates a Vulnerability in CMGs for Therapeutic Intervention. Bioessays 2020; 42(4):e1900218 doi 10.1002/bies.201900218.
47. Donnelly A, Blagg B S. Novobiocin and additional inhibitors of the Hsp90 C-terminal nucleotide-binding pocket. Curr Med Chem 2008; 15(26):2702-17 doi 10.2174/092986708786242895.
48. Marcu M G, Schulte T W, Neckers L. Novobiocin and related coumarins and depletion of heat shock protein 90-dependent signaling proteins. J Natl Cancer Inst 2000; 92(3):242-8 doi 10.1093/jnci/92.3.242.
49. Zhou J, Gelot C, Pantelidou C, Li A, Yucel H, Davis R E, et al. A first-in-class Polymerase Theta Inhibitor selectively targets Homologous-Recombination-Deficient Tumors. Nat Cancer 2021; 2(6):598-610 doi 10.1038/s43018-021-00203-x.
50. Godfrey J C, Price K E. Structure-activity relationships in coumermycins. Adv Appl Microbiol 1972; 15:231-96 doi 10.1016/s0065-2164(08)70094-7.
51. Mukherjee P, Cao T V, Winter S L, Alexandrow M G. Mammalian MCM loading in late-G(1) coincides with Rb hyperphosphorylation and the transition to post-transcriptional control of progression into S-phase. PLoS ONE 2009; 4(5):e5462.
52. Alexandrow M G, Hamlin J L. Chromatin decondensation in S-phase involves recruitment of Cdk2 by Cdc45 and histone H1 phosphorylation. J Cell Biol 2005; 168(6):875-86.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various aspects, the terms “consisting essentially of” and “consisting of” can be used in place of “comprising” and “including” to provide for more specific aspects of the disclosure and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound of Formula I 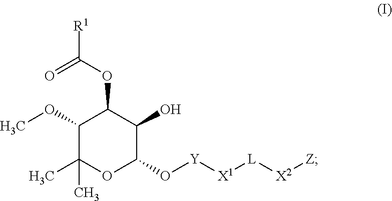
or a pharmaceutically acceptable salt thereof;
wherein:
R¹ is selected from monocyclic or bicyclic heteroaryl and monocyclic or bicyclic heterocycloalkyl, wherein each R¹ optionally includes at least one ring nitrogen atom substituted with R⁷ as allowed by valency, and wherein R¹ is optionally substituted with 1, 2, 3, or 4 groups selected from R⁸ as allowed by valency;
Y is selected from a bond, bicyclic aryl, or bicyclic heteroaryl, wherein Y is optionally substituted with 1, 2, 3, or 4 groups independently selected from R² as allowed by valency;
X¹ is selected from a bond or —NR^{a}—;
L is selected from a bond or -L¹-L²-L³-L⁴-L⁵-L⁶-L⁷-;
L¹, L², L³, L⁴, L⁵, L⁶, and L⁷ are independently selected from:
a) a bond;
b) —C(═O)—;
c) —C≡C—;
d) —NR^{a}—;
e) —O—;
d) C₁-C₁₀ alkyl;
e) cycloalkyl optionally substituted with 1, 2, 3, or 4 substituents independently selected from R³;
f) heterocycloalkyl optionally substituted with 1, 2, 3, or 4 substituents independently selected from R⁴;
g) aryl optionally substituted with 1, 2, 3, or 4 groups independently selected from R⁵;
h) heteroaryl optionally substituted with 1, 2, 3, or 4 groups independently selected from R⁶;
i) —NR^{a}(C═O)—;
j) —C(═O)NR^{a}—;
k) —C(═O)(C₁-C₆ alkyl)-;
l) —(C₁-C₆ alkyl)C(═O)—;
m) —(C₁-C₆ alkyl)NR^{a}—;
n) —NR^{a}(C₁-C₆ alkyl)-;
o) —(C₁-C₆ alkyl)O—; and
p) —O(C₁-C₆ alkyl)-;
X² is selected from a bond, —NR^{a}—, —O—, —C≡C—, —C(═O)—, —S(═O)₂—, —C(═O)NR^{a}—, —NR^{a}C(═O)—, —S(═O)₂NR^{a}—, —NR^{a}S(═O)₂—, and —NR^{a}(C═O)NR^{b}—;
Z is an E3 ubiquitin ligase binding moiety;
R² is selected at each occurrence from halogen, hydroxy, nitro, —NHR^{a}, C₁-C₆ alkyl, C₁-C₆ haloalkyl, aryl, and oxo;
R³ and R⁴ are independently selected at each occurrence from halogen, hydroxy, —NHR^{a}, C₁-C₆ alkyl, C₁-C₆ haloalkyl and oxo;
R⁵ and R⁶ are independently selected at each occurrence from halogen, hydroxy, nitro, —NHR^{a}, C₁-C₆ alkyl, C₁-C₆ haloalkyl, and aryl;
R⁷ is selected from: hydrogen; C₁-C₄ alkyl; C₂-C₆ alkenyl; C₂-C₆ alkynyl; C₃-C₆ cycloalkyl; C₁-C₆ alkoxy; and (Co-C₆ alkyl)(aryl) which is optionally substituted with halogen, C₁-C₆ alkyl, or C₁-C₆ alkoxy;
R⁸ is selected from: hydrogen; halogen; hydroxy; —NR^{a}R^{b}; nitro; C₁-C₆ alkyl; C₁-C₆ haloalkyl; C₃-C₆ cycloalkyl; C₃-C₆ heterocycloalkyl; C₂-C₆ alkenyl; C₂-C₆ alkynyl; C₁-C₆ alkoxy; (C₀-C₆ alkyl)(aryl) which is optionally substituted with halogen, C₁-C₆ alkyl, or C₁-C₆ alkoxy; and (Co-C₆ alkyl)(heteroaryl) which is optionally substituted with halogen, C₁-C₆ alkyl, or C₁-C₆ alkoxy; and
R^{a} and R^{b} are independently selected at each occurrence from hydrogen and C₁-C₆ alkyl.

2. The compound of claim 1, wherein Z is selected from:

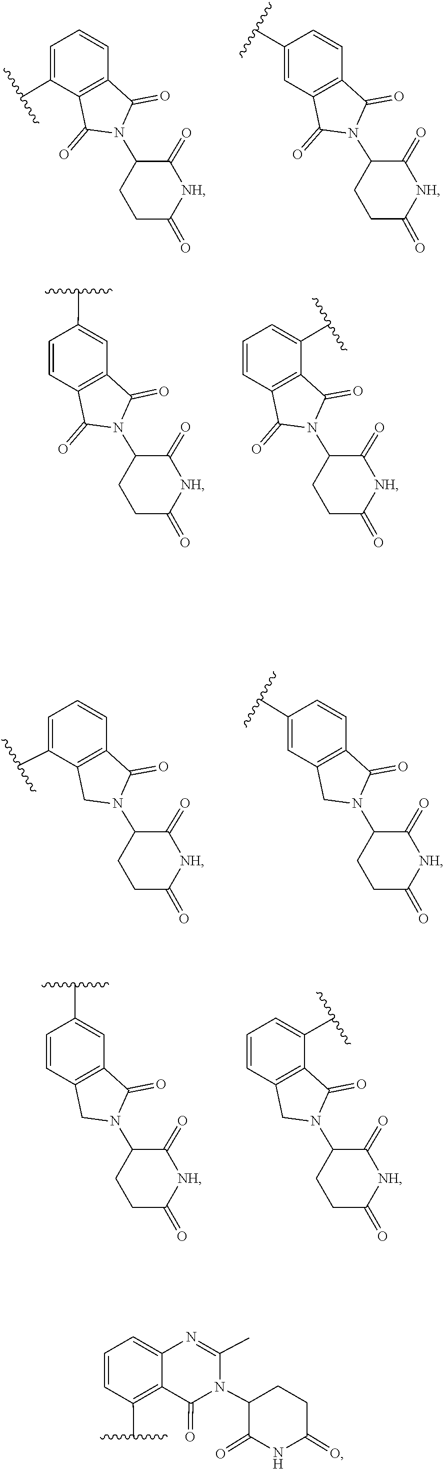

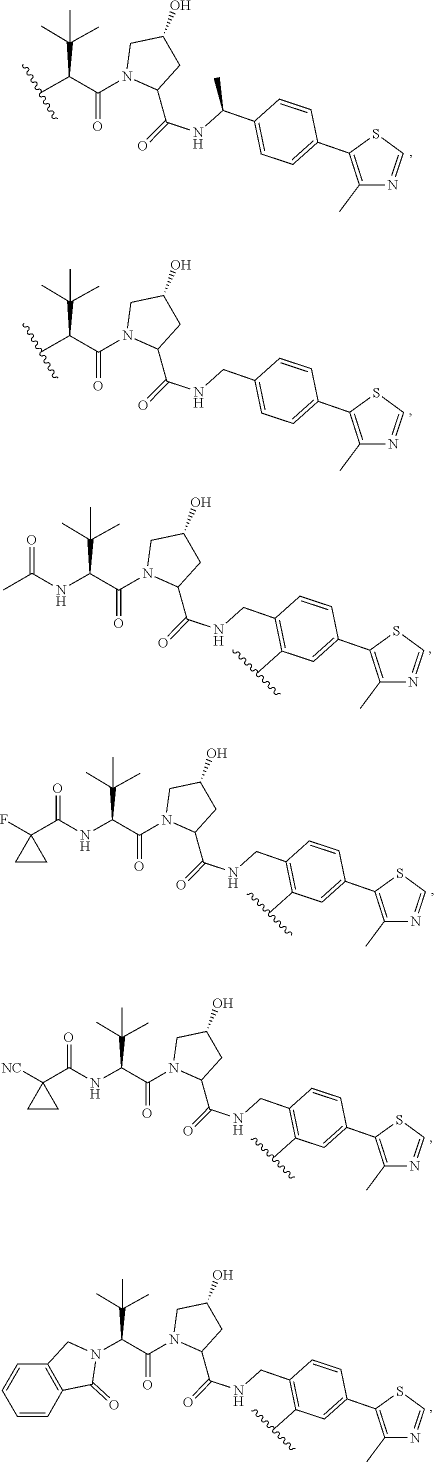 [SMILES: *[C@H](C(=O)N1C[C@H](O)CC1C(=O)N[C@@H](C)c1ccc(-c2scnc2C)cc1)C(C)(C)C ; *[C@H](C(=O)N1C[C@H](O)CC1C(=O)NCc1ccc(-c2scnc2C)cc1)C(C)(C)C ; *c1cc(-c2scnc2C)ccc1CNC(=O)C1C[C@@H](O)CN1C(=O)[C@@H](NC(C)=O)C(C)(C)C ; *c1cc(-c2scnc2C)ccc1CNC(=O)C1C[C@@H](O)CN1C(=O)[C@@H](NC(=O)C1(F)CC1)C(C)(C)C ; *c1cc(-c2scnc2C)ccc1CNC(=O)C1C[C@@H](O)CN1C(=O)[C@@H](NC(=O)C1(C#N)CC1)C(C)(C)C ; *c1cc(-c2scnc2C)ccc1CNC(=O)C1C[C@@H](O)CN1C(=O)[C@@H](N1Cc2ccccc2C1=O)C(C)(C)C]

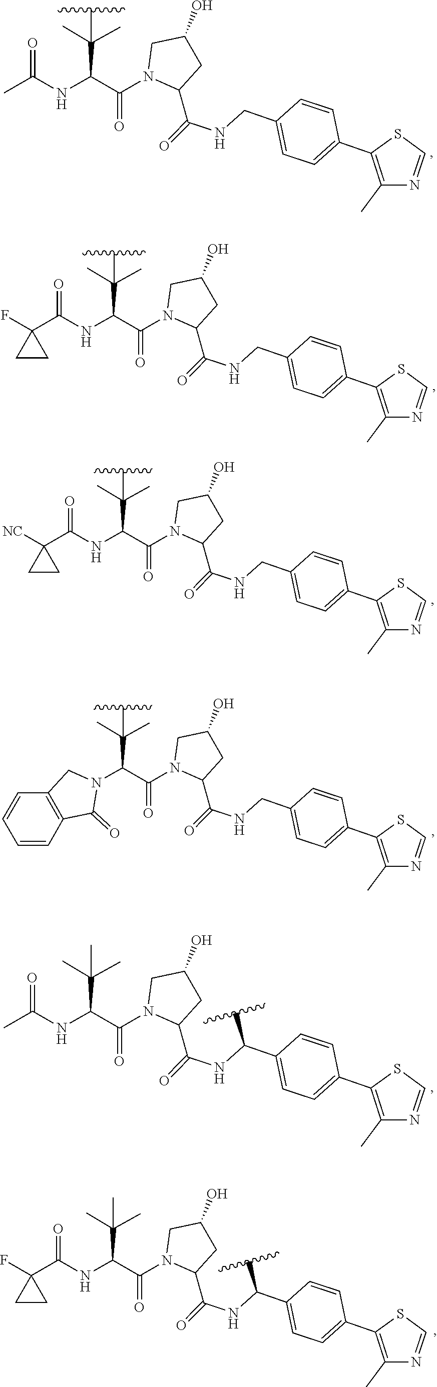 [SMILES: *C(C)(C)[C@H](NC(C)=O)C(=O)N1C[C@H](O)CC1C(=O)NCc1ccc(-c2scnc2C)cc1 ; *C(C)(C)[C@H](NC(=O)C1(F)CC1)C(=O)N1C[C@H](O)CC1C(=O)NCc1ccc(-c2scnc2C)cc1 ; *C(C)(C)[C@H](NC(=O)C1(C#N)CC1)C(=O)N1C[C@H](O)CC1C(=O)NCc1ccc(-c2scnc2C)cc1 ; *C(C)(C)[C@@H](C(=O)N1C[C@H](O)CC1C(=O)NCc1ccc(-c2scnc2C)cc1)N1Cc2ccccc2C1=O ; *[C@H](NC(=O)C1C[C@@H](O)CN1C(=O)[C@@H](NC(C)=O)C(C)(C)C)c1ccc(-c2scnc2C)cc1 ; *[C@H](NC(=O)C1C[C@@H](O)CN1C(=O)[C@@H](NC(=O)C1(F)CC1)C(C)(C)C)c1ccc(-c2scnc2C)cc1]

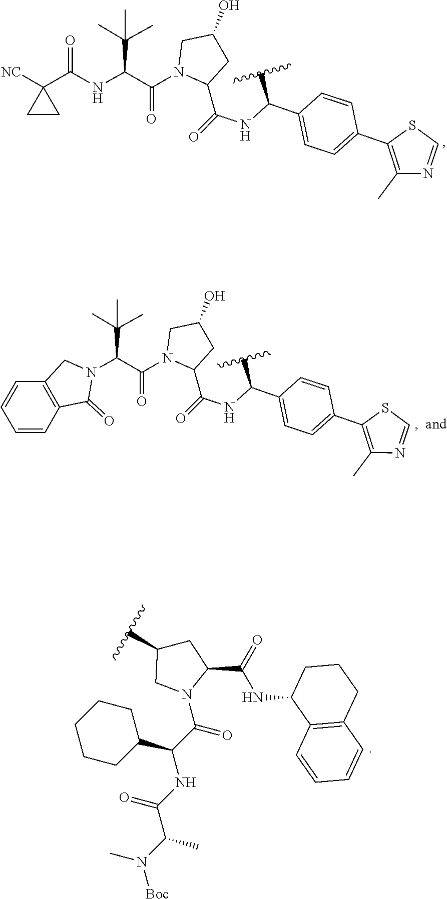 [SMILES: *[C@H](NC(=O)C1C[C@@H](O)CN1C(=O)[C@@H](NC(=O)C1(C#N)CC1)C(C)(C)C)c1ccc(-c2scnc2C)cc1 ; *[C@H](NC(=O)C1C[C@@H](O)CN1C(=O)[C@@H](N1Cc2ccccc2C1=O)C(C)(C)C)c1ccc(-c2scnc2C)cc1 ; *[C@H]1C[C@@H](C(=O)N[C@@H]2CCCc3ccccc32)N(C(=O)[C@@H](NC(=O)[C@H](C)N(C)C(=O)OC(C)(C)C)C2CCCCC2)C1]

3-5. (canceled)

6. The compound of claim 1, wherein R¹ is 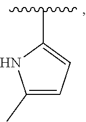 [SMILES: *c1ccc(C)[nH]1]
or wherein R¹ is selected from:

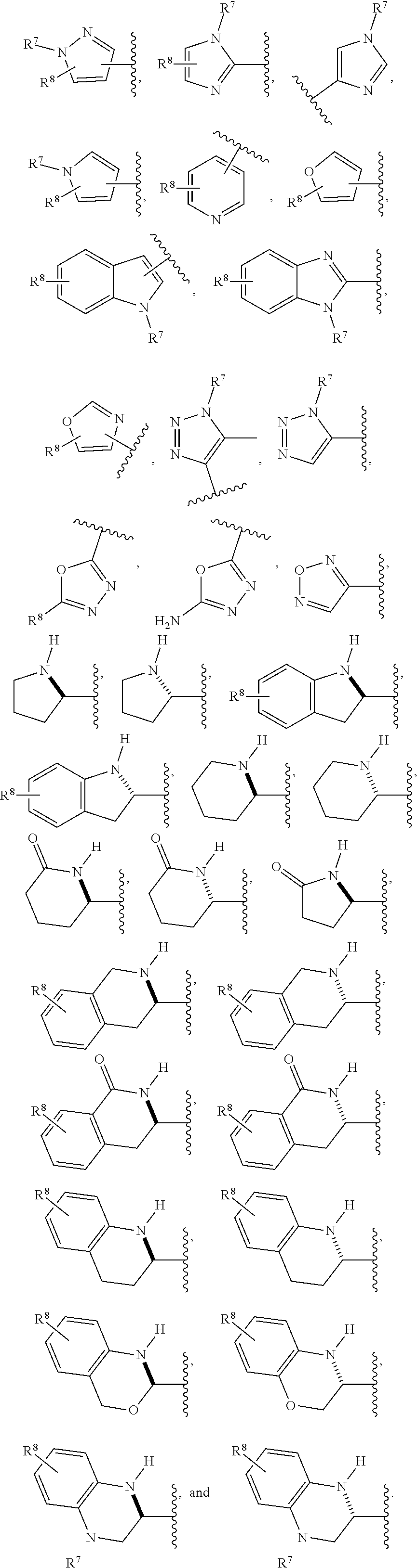

7-8. (canceled)

9. The compound of claim 1, wherein Y is a bond.

10. The compound of claim 1,
wherein Y is 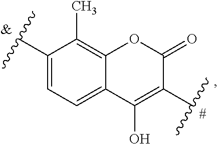 [SMILES: *c1ccc2c(O)c(*)c(=O)oc2c1C]
or
wherein Y is selected from: 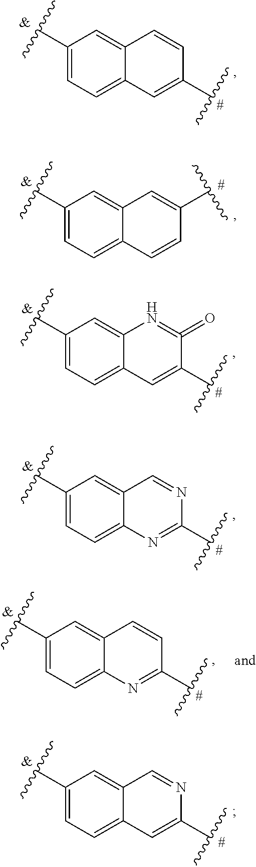
wherein & denotes the point of attachment to the neighboring oxygen atom and # denotes the point of attachment to X¹ and/or X².

11. (canceled)

12. The compound of claim 1,
wherein X¹ is —NH—.

13. The compound of any one of claims 1-12, wherein Lt, L², L³, L⁴, L⁵, L⁶, and L⁷ are each independently selected from: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—; 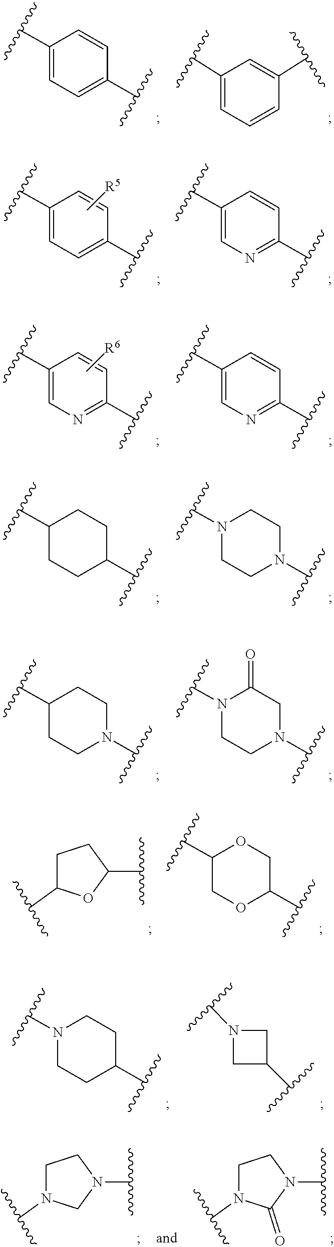
wherein L¹, L², L³, L⁴, L⁵, L⁶, and L⁷ are selected in such a way that: no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of L¹, L², L³, L⁴, L⁵, L⁶, and L⁷.

14. The compound of claim 1, wherein L is selected from: 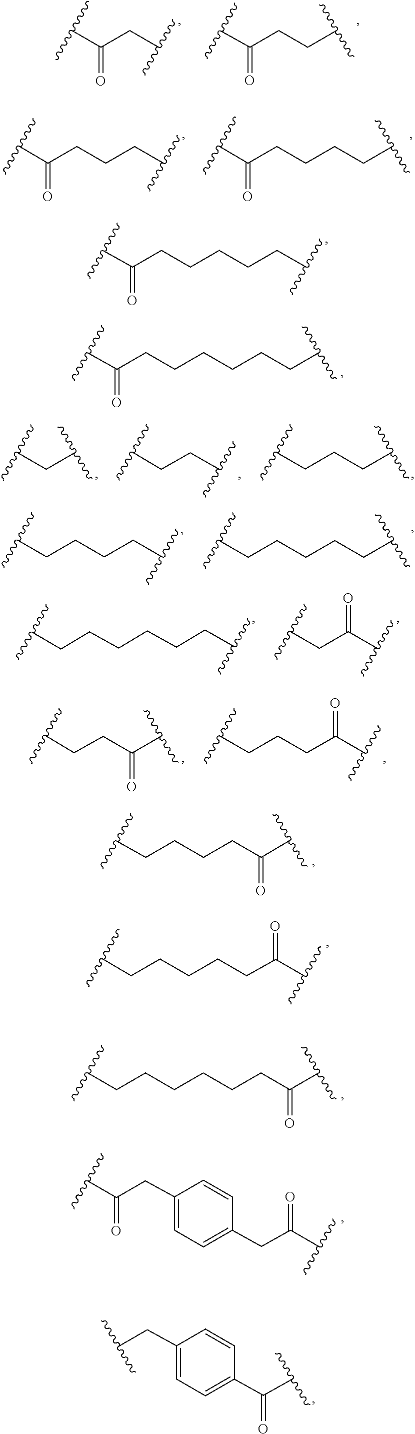 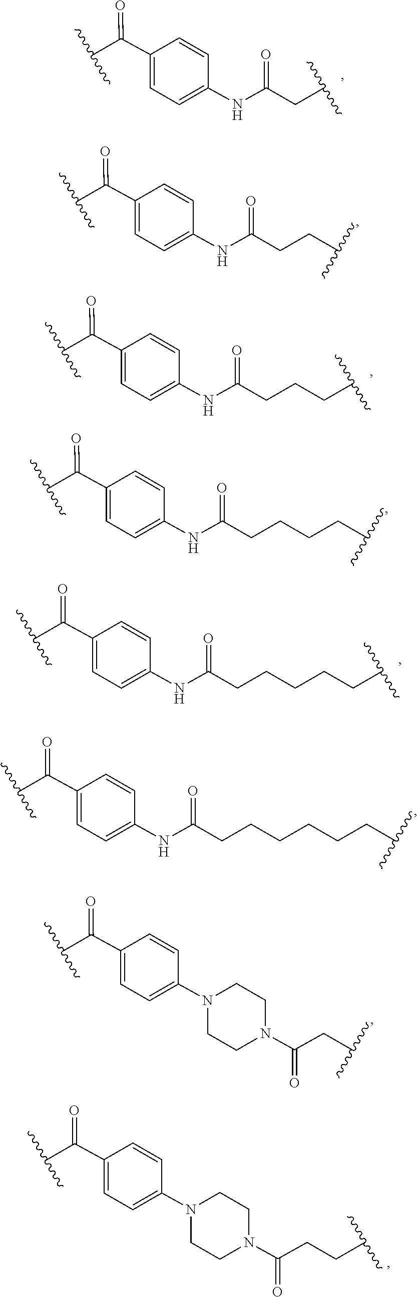 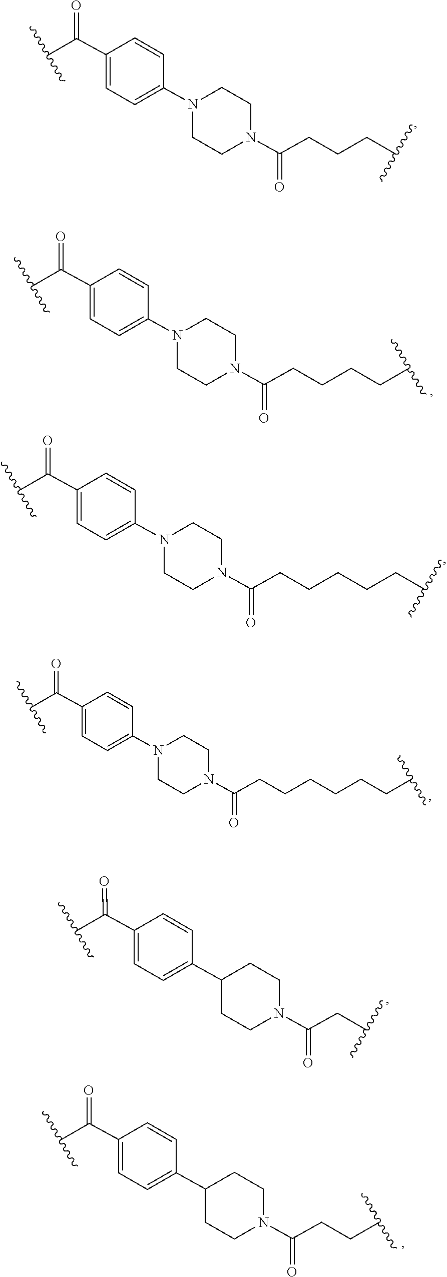 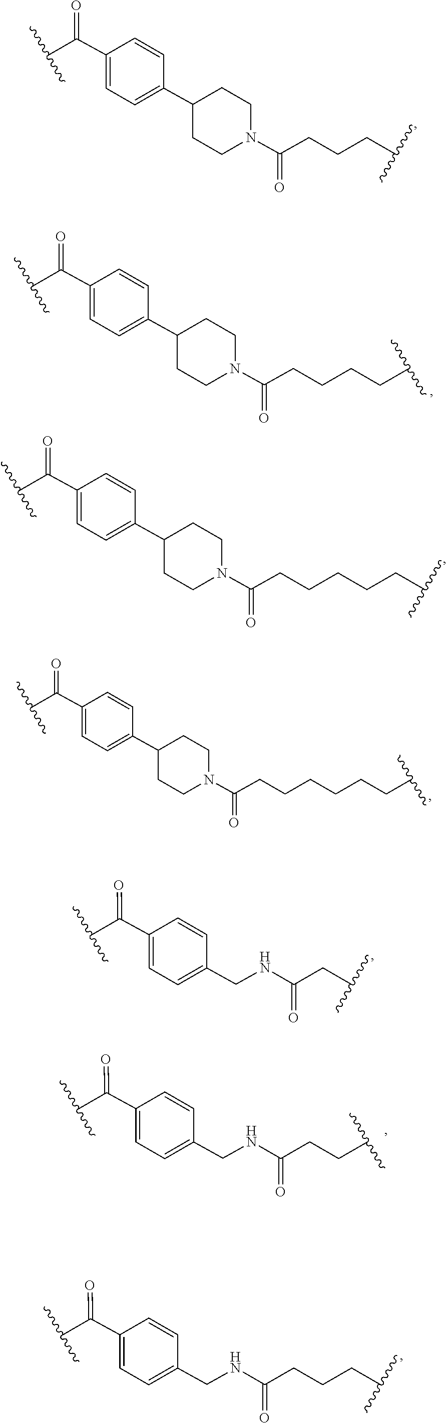 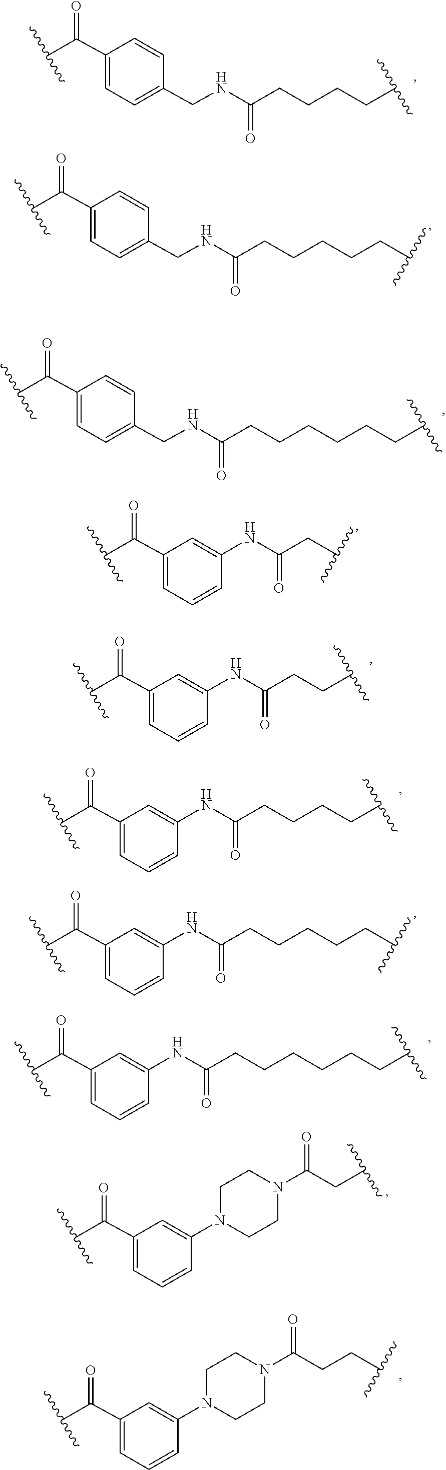 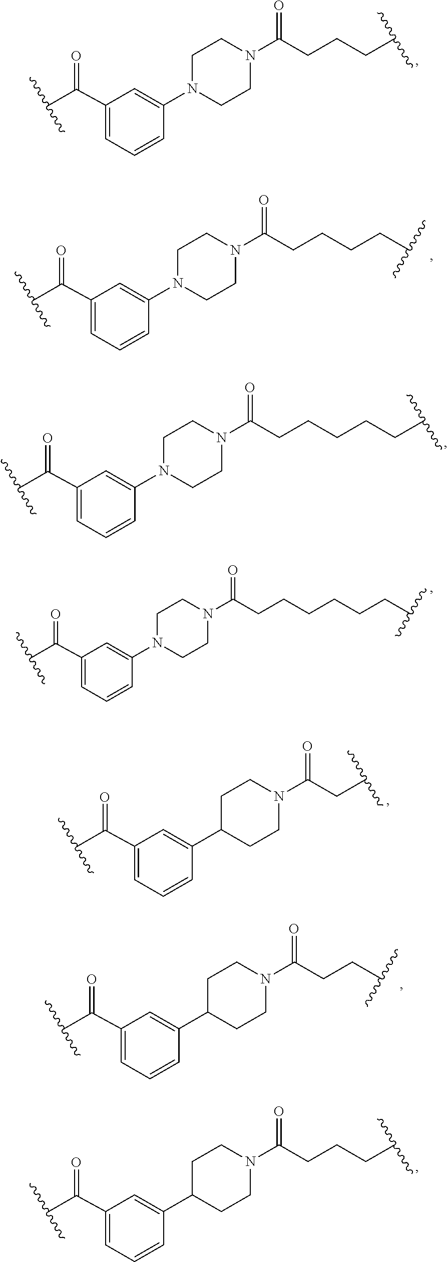 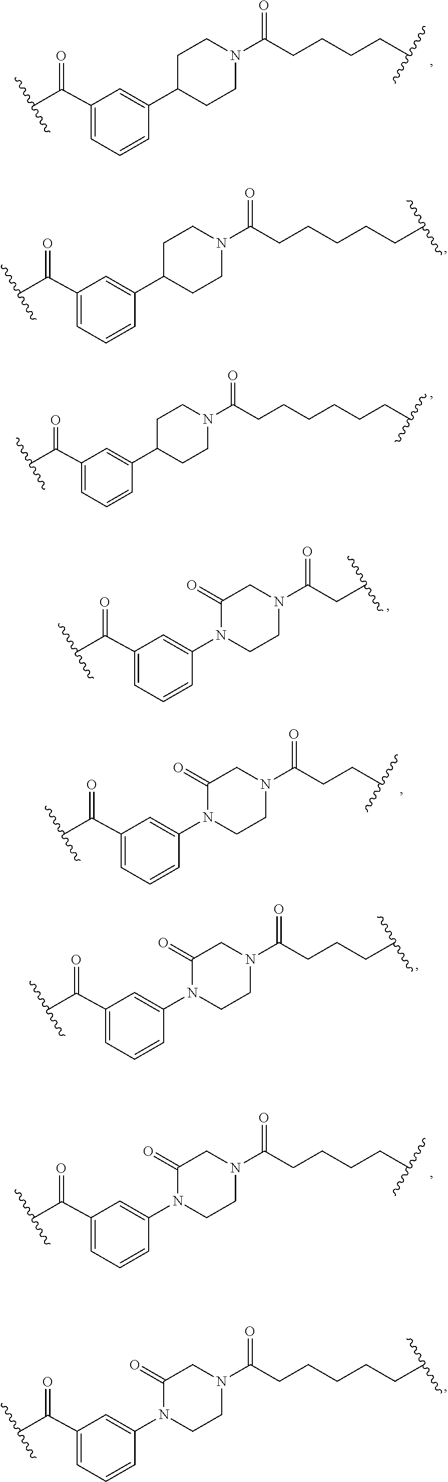 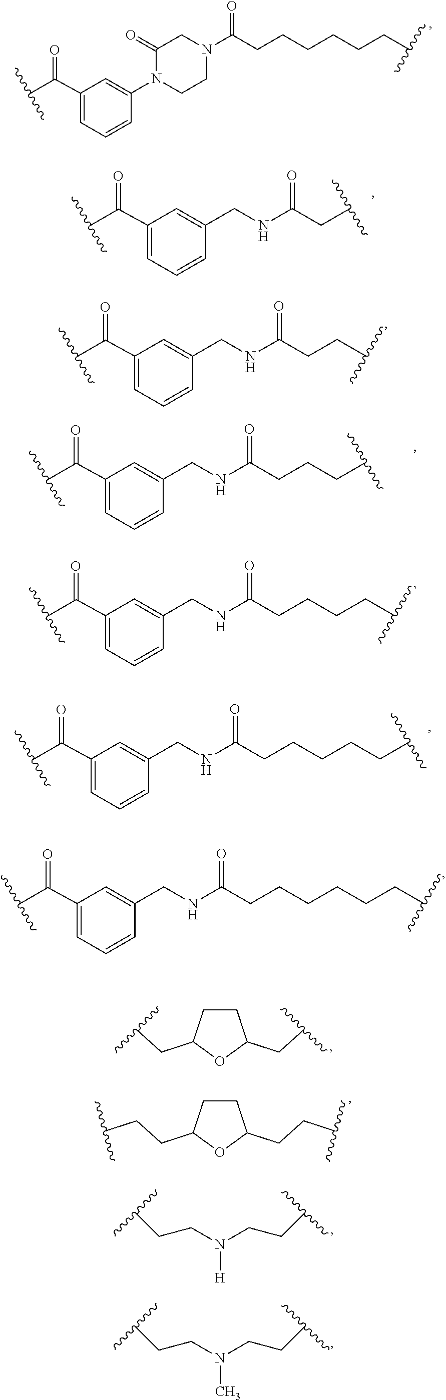 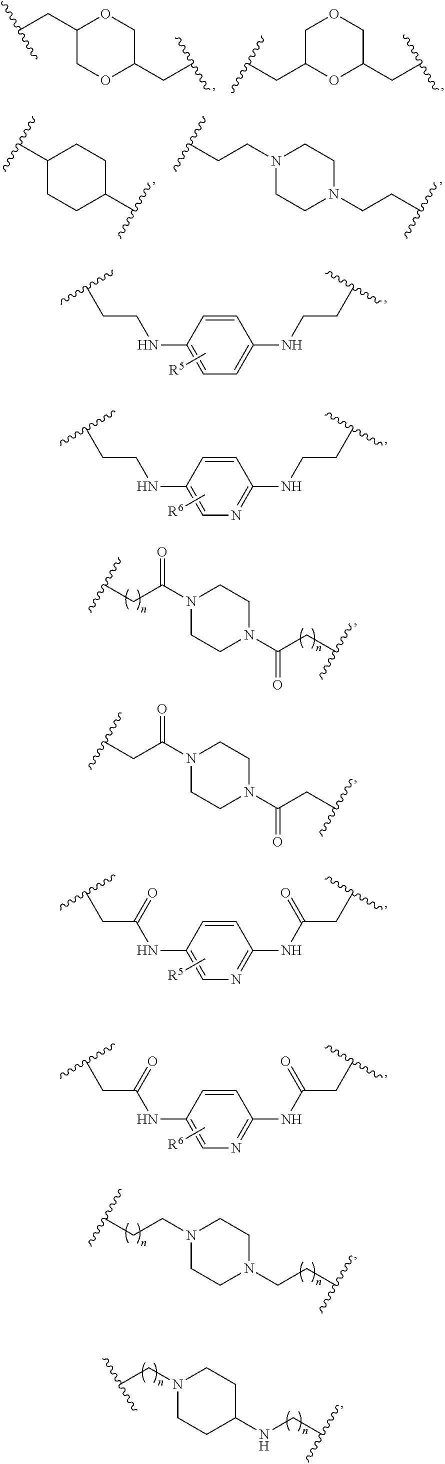 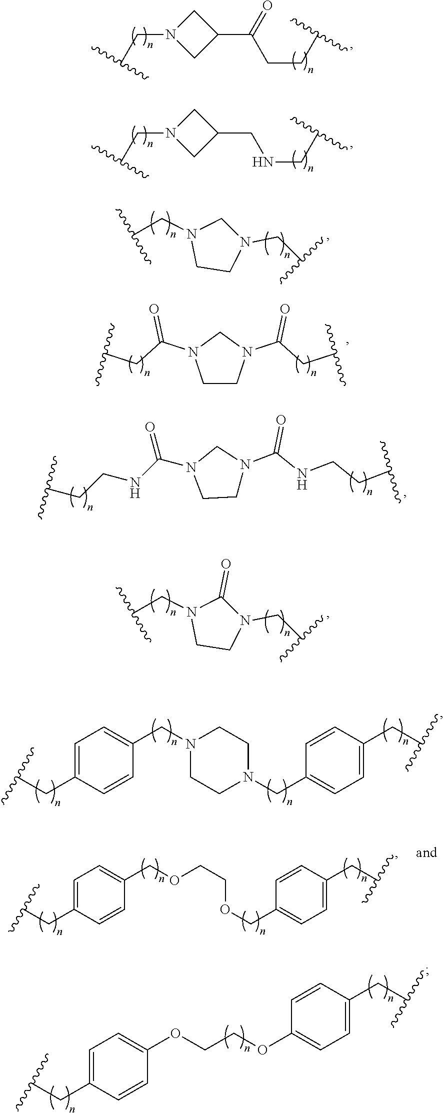
wherein n is independently selected at each occurrence from 1, 2, 3, 4, 5, and 6.

15-28. (canceled)

29. The compound of claim 1, wherein X² is a bond.

30. The compound of claim 1, wherein X² is —NH—, —O—, —C≡C—, —C(═O)—, —S(═O)₂—, —C(═O)NH—, —NHC(═O)—, —S(═O)₂NH—, —NHS(═O)₂—, and —NH(C═O)NH—.

31-39. (canceled)

40. A pharmaceutical composition comprising a compound of claim 1, or a pharmaceutically acceptable salt thereof, and a pharmaceutically acceptable carrier or excipient.

41. A method for treating cancer in a subject in need thereof comprising administering to the subject a therapeutically effective amount of a compound of claim 1, or a pharmaceutically acceptable salt thereof.

42-49. (canceled)

50. A method for treating cancer in a subject in need thereof comprising:
(a) determining whether the cancer is associated with elevated expression of Myc and/or elevated expression of Cyclin E; and
(b) if the cancer is determined to be associated with elevated expression of Myc and/or elevated expression of Cyclin E in (a), administering a therapeutically effective amount of a compound of claim 1, or a pharmaceutically acceptable salt thereof.

51. A method for treating cancer in a subject in need thereof, wherein the cancer has been previously determined to be associated with elevated expression of Myc and/or elevated expression of Cyclin E, the method comprising administering a therapeutically effective amount of a compound of claim 1, or a pharmaceutically acceptable salt thereof.

52-54. (canceled)

55. A method of treating an infection with a papillomavirus in a subject in need thereof comprising administering a therapeutically effective amount of a compound of claim 1, or a pharmaceutically acceptable salt thereof.

56-60. (canceled)

61. A method for degrading a helicase in a eukaryotic cell comprising contacting the cell with an effective amount of a compound of claim 1, or a pharmaceutically acceptable salt thereof.

62-65. (canceled)

66. A method for inhibiting replication of a papillomavirus in a eukaryotic cell comprising contacting the cell with an effective amount of a compound of claim 1, or a pharmaceutically acceptable salt thereof.

67-70. (canceled)

71. A method for treating cancer in a subject in need thereof comprising:
(a) determining whether the cancer harbors one or more inherited or acquired germ-line mutations; and
(b) if the cancer is determined to harbor one or more inherited or acquired germ-line mutations in (a), administering to the subject a therapeutically effective amount of a compound of claim 1, or a pharmaceutically acceptable salt thereof.

72. A method for treating cancer in a subject in need thereof, wherein the cancer has been previously determined to harbor one or more inherited or acquired germ-line mutations, the method comprising administering to the subject a therapeutically effective amount of a compound of claim 1, or a pharmaceutically acceptable salt thereof.

73-75. (canceled)

76. A method for treating cancer in a subject in need thereof comprising:
(a) administering to the subject a therapeutically effective amount of a compound of claim 1, or a pharmaceutically acceptable salt thereof; and
(b) administering an additional therapeutic agent selected from a Chk1 inhibitor, an ATR inhibitor, a Cdc7 inhibitor, and a Parp inhibitor.
